(19) United States
(12) Patent Application Publication
(10) Pub. No.: US 2023/0364111 A1
(43) Pub. Date: Nov. 16, 2023

(54) METHODS AND COMPOSITIONS FOR TREATING VIRAL INFECTIONS

(71) Applicants: MAX BioPharma, Inc., Los Angeles, CA (US); Japan as represented by Director-General of National Institute of Infectious Diseases, Tokyo (JP)

(72) Inventors: Farhad Parhami, Los Angeles, CA (US); Frank Stappenbeck, Los Angeles, CA (US); Koichi Watashi, Tokyo (JP); Hirofumi Ohashi, Tokyo (JP)

(73) Assignees: MAX BioPharma, Inc., Los Angeles, CA (US); Japan as represented by Director-General of National Institute of Infectious Diseases, Tokyo (JP)

(21) Appl. No.: 18/028,709

(22) PCT Filed: Oct. 7, 2021
(86) PCT No.: PCT/US2021/054049

§ 371 Date: Mar. 27, 2023

Related U.S. Application Data
(60) Provisional application No. 63/089,288, filed on Oct. 8, 2020.

(51) Int. Cl. A61K 31/575; A61K 45/06; A61P 31/14

(52) U.S. Cl. CPC A61K 31/575; A61K 45/06; A61P 31/14

(57) ABSTRACT

Described herein are methods and compositions for treating viral infections.

### CROSS-REFERENCE TO RELATED APPLICATIONS

This application claims benefit under 35 U.S.C. § 119(e) of U.S. Provisional Application No. 63/089,288, filed Oct. 8, 2020, content of which is incorporated herein by reference in their entirety.

### SEQUENCE LISTING

The instant application contains a Sequence Listing which has been submitted electronically in ASCII format and is hereby incorporated by reference in its entirety. Said ASCII copy, created on Oct. 7, 2021, is named SequenceListing-088907-000001WO00_SL.txt and is 2,484 bytes in size.

### TECHNICAL FIELD

The present disclosure relates to methods and compositions for treating viral infections.

### BACKGROUND

Viral infections are responsible for many acute and chronic life-threatening diseases and account for a very large fraction of infectious disease mortality and morbidity worldwide. One of the most effective treatments of viral diseases is use of antiviral drugs. Different antiviral drugs target different stages of the viral life cycle. Despite extensive efforts, the development of effective anti-viral drugs has largely been empirical. Further, as virus strains change over time, the emergence of resistant mutants further diminishes the anti-viral activity of existing anti-viral agents. There remains a critical and unmet medical need for new therapeutic modes of treating viral infections. Therefore, there is a need in the art for new treatments for viral infections.

### SUMMARY

Various aspects described herein are based on the unexpected and surprising discovery that oxysterols of Formula (I) inhibit viral entry into cells and/or viral replication, and have potent anti-viral activity.

Described herein are oxysterol-based compounds that can significantly reduce or prevent hepatitis B virus (HBV) or hepatitis D virus (HDV) infection in cells. Exemplary compounds, such as Oxy45, Oxy181, Oxy185, Oxy220, and Oxy229 significantly reduced HBV infection by preventing the internalization of the HBV from the cell surface. Such compounds can be useful as drug treatments for HBV and HDV infection as well as other similar DNA or RNA viruses.

Also described herein are oxysterol-based compounds that can reduce or prevent the infection of cells with SARS-CoV-2. Exemplary compounds, such as Oxy210, Oxy211, Oxy221 and Oxy232 drastically reduced SARS-CoV-2 replication. Such compounds can be useful as drug treatments for SARS-CoV-2 infection as well as other viruses including other coronaviruses and other RNA viruses.

Accordingly, in one aspect provided herein is a method for treating a viral infection in a subject. Generally, the method comprises administering a therapeutically effective amount of a compound of Formula (I), or a pharmaceutically acceptable salt or solvate thereof, to a subject in need thereof. The compound of Formula (I) is of chemical structure:

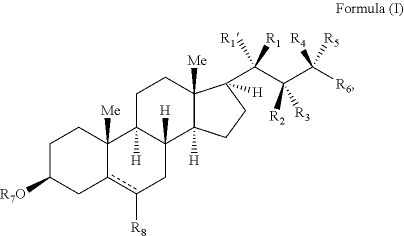

wherein:
[symbol] is a single or double bond;
R₁ and R₁′ are independently hydrogen, substituted or unsubstituted C₁-C₈ alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl, provided that one of R₁ and R₁′ is OH or R₁ and R₁′ together are ═O;
R₂, R₃, R₄, and R₅ are independently hydrogen, deuterium, C₁-C₈alkyl, or —OH, or one of R₂ or R₃ together with one of R₄ or R₅ forms a double bond;
R₆ is alkyl, aryl or heteroaryl, wherein the alkyl, aryl or the heteroaryl are optionally substituted with 1, 2, 3, or 4 R₉ groups;
R₇ is hydrogen, substituted or unsubstituted C₁-C₈alkyl, or —C(O)NR₁₀R₁₁;
R₈ is hydrogen or —OH;
each R₉ is independently selected from deuterium, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₂₋₉heteroaryl, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR₁₂, —SR₁₂, —N(R₁₃)(R₁₃), —C(O)OR₁₃, —C(O)N(R₁₃)(R₁₄), —C(O)R₁₅, —S(O)₂R₁₅, and —S(O)₂N(R₁₃)(R₁₄);
R₁₀ and R₁₁ are independently hydrogen, substituted or unsubstituted C₁-C₈alkyl, or substituted or unsubstituted aryl;
each R₁₂ is independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl;
each R₁₃ and each R₁₄ are each independently selected from H, C₁₋₆alkyl, C₁₋₆haloalkyl, C₃₋₆cycloalkyl, C₂₋₉heterocycloalkyl, C₆₋₁₀aryl, and C₁₋₆heteroaryl; and
each R₁₅ is independently selected from C₁₋₆alkyl, C₁₋₆haloalkyl, C₃₋₆cycloalkyl, C₂₋₉heterocycloalkyl, C₆₋₁₀aryl, and C₁₋₉heteroaryl.

The methods and compositions described herein can be used for treating any number of viral infections. For example, the methods and compositions described herein can be used for treating a hepatitis virus infection, e.g., a hepatitis B virus (HBV) or hepatitis D virus (HDV) infection.

In some embodiments of any one of the aspects, the methods and compositions described herein can be useful in treating a coronavirus infection, e.g., severe acute respiratory syndrome coronavirus 2 (SARS-CoV-2) infection.

It is noted that administering to the cell can be in vitro or in vivo. For example, when the administering to the cell is in vivo, the compound can be administered to a subject. The subject can be one having a viral infection or in need of treatment for a viral infection, or as prophylaxis to prevent viral infection.

### BRIEF DESCRIPTION OF THE DRAWINGS

FIG. 1A is a schematic representation of an assay for determining effect of the compounds on HBV infection. HepG2-hNTCP-C4 cells pretreated with the compounds for 3 h were inoculated with HBV in the presence of compounds (100 nM preS1 peptide or 33 μM the compound) for 16 h. After washing out free virus and compounds, HBs in the culture supernatant at 12 days post-inoculation was measured by ELISA to evaluate HBV infection. Cell viability was measured by MTT assay. PreS1-peptide was used as a positive control that is known to inhibit HBV entry into cells.

FIGS. 1B and 1C are bar graphs showing the effect of exemplary compounds of Formula (I) on HBV infection (FIG. 1B) and cell viability (FIG. 1C).

FIG. 2A is a schematic representation of an assay for determining the anti-HBV effect of the compounds at different concentrations. HepG2-hNTCP-C4 cells were infected with HBV and treated with the compounds at 5 or 10 μM as shown in FIG. 1A.

FIGS. 2B-2C are bar graphs showing the anti-HBV effect of exemplary oxy45, oxy185, oxy181, oxy220, and oxy189 at different concentrations on HBV infection (FIGS. 2B and 2C) and cell viability (FIG. 2D). Data are shown as mean±SD. Statistical significance was determined using a two-tailed non-paired Student's t-test (*P<0.05, **P<0.01). In this assay, oxy45, oxy185, oxy181, and oxy220 were identified to show strong anti-HBV activities, whereas oxy189 showed little anti-HBV activity.

FIG. 3A is a schematic representation of an immunofluorescence assay for detecting HBc to determine the anti-HBV effect of the compounds. HepG2-hNTCP-C4 cells were infected with HBV and treated with the compounds at 10 μM as shown in FIG. 2A.

FIG. 3B shows exemplary compounds oxy45, oxy181, oxy185, oxy220, and oxy229 reduced HBV infection as monitored by the expression of HBc in the cells. Red and blue signals indicate HBc and the nucleus, respectively.

FIG. 4A is a schematic representation of an assay for determining the effect of the compounds on HBV replication. Hep38.7-Tet cells, which replicate HBV but do not support HBV entry, were treated with the compounds (1.1, 3.3, and 10 μM compound, or 1 μM entecavir (ETV) as a positive control that inhibits HBV replication) for 6 days and HBV replication was monitored by quantifying intracellular HBV DNA by real-time PCR.

FIG. 4B is a schematic representation of HBV life cycle. Purple box shows the replication step measured by the assay shown in FIG. 4A.

FIG. 4C is a bar graph showing exemplary compounds oxy45, oxy185 and oxy189 did not inhibit HBV replication.

FIG. 5A is a schematic representation of an assay for determining the effect of the compounds on HBV preS1 attachment. HepG2-hNTCP-C4 cells were exposed to TAMRA-labeled HBV preS1 peptide for 30 min to mimic HBV-cell attachment in the absence or presence of the compounds (30 μM oxysterols or 100 nM non-labeled preS1 peptide). The cells were washed out, fixed with 4% paraformaldehyde, stained with DAPI, and observed with fluorescence microscopy

FIG. 5B is a schematic representation of HBV life cycle. Blue box shows the HBV attachment step measured by the assay shown in FIG. 5A

FIG. 5C are fluorescence microscopy photographs showing exemplary compounds oxy45, oxy185, and oxy189, did not inhibit HBV preS1 attachment to host cells. Red and blue signals indicate TAMRA-labeled preS1 peptide and the nucleus, respectively.

FIG. 6A is a schematic representation of an assay for determining the effect of the compounds on HBV internalization. HepG2-hNTCP-C4 cells were treated with TAMRA-labeled HBV preS1 peptide for 30 min at 4° C. After washing, the cells were then transferred to 37° C. to allow virus internalization in the presence or absence of the compounds for 8 h and were observed by confocal microscopy.

FIG. 6B is a schematic representation of HBV life cycle. Blue box shows the HBV internalization step measured by the assay shown in FIG. 6A

FIG. 6C are confocal microscopy photographs showing exemplary compounds oxy45 and oxy185, but not oxy189, inhibited HBV preS1 internalization from the cell surface. Troglitazone was used as a positive control as it was reported to inhibit HBV internalization.

FIG. 7A is a schematic representation of an assay for determining effect of the compounds on HDV infection. HepG2-hNTCP-C4 cells pretreated with the compounds for 3 h were inoculated with HDV in the presence or absence of the compounds at 10 μM for 16 h. After washing out, the cells were cultured for another 6 days and were detected for intracellular HDAg by immunofluorescence.

FIG. 7B shows exemplary compounds oxy45, oxy181, oxy185, but not oxy189, reduced HDV infection. Red and blue signal show HDAg and the nucleus.

FIGS. 8A-8E show oxysterols inhibit SARS-CoV-2 infection. (FIG. 8A) Schematic model of the SARS-CoV-2 infection assay. VeroE6 cells overexpressing transmembrane protease, serine 2 (TMPRSS2) were inoculated with SARS-CoV-2 in the presence of compounds for 1 h, followed by washing out the free virus and incubating the cells with the compounds for 24 or 48 h. Viral RNA in the culture supernatant and viral N protein in the cells was quantified at 24 h post-inoculation by real-time RT-PCR and immunofluorescence analyses, respectively. Cytopathic effects (CPE) were viewed under a microscope at 48 h post-inoculation. Solid and dashed boxes indicate the periods that the cells were treated with and without the compounds or the virus, respectively. (FIG. 8B) Images of the cells treated with the virus in the presence of dimethyl sulfoxide (DMSO), 10 μM Remdesivir (RDV), 30 μM cholesterol, or 30 μM 7-ketocholesterol (7-KC). Scale bar, 100 μm. (FIG. 8C) Viral N protein in the cells was detected by indirect immunofluorescence analysis. The red and blue signals represent viral N protein and nuclei, respectively. Scale bar, 50 μm. (FIG. 8D) Dose-response curves for SARS-CoV-2 RNA upon treatment with the compounds as indicated. OHC: hydroxycholesterol. Viral RNAs in the culture supernatant were quantified by real-time RT-PCR and plotted against compound concentrations up to 30 μM. (FIG. 8E) Viability of cells treated with compounds as indicated for 24 h was quantified using an MTT assay.

FIGS. 9A-9G show Oxy210, an oxysterol derivative, potently inhibits the SARS-CoV-2 propagation and alleviates the virus-induced CPE. (FIG. 9A) Virus-induced CPE was examined in the cells inoculated with the virus in the presence of DMSO, 10 μM RDV, 10 μM Oxy133, or 10 μM Oxy210. Scale bar, 100 μm. (FIG. 9B) Viral N protein in the cells was detected by immunofluorescence analysis as described in FIG. 8C. Scale bar, 50 μm. (FIG. 9C) Dose-response curves for viral RNA upon treatment with oxysterol derivatives as indicated. The secreted viral RNA in the culture supernatant at 24 h post-inoculation was quantified by real-time RT-PCR and plotted against compound concentration. The chemical structures of oxysterols are also shown above the graphs. (FIG. 9D) Viability of cells treated with the compounds was quantified using an MTT assay. (FIGS. 9E and 9F) Inhibitory effects toward transforming growth factor β (TGFβ) and hedgehog (Hb) signaling. NIH3T3 cells pretreated with or without 10 μM Oxy210 or 10 μM Oxy232 for 2 h were stimulated with 20 ng/mL TGFβ (FIG. 9E) or with conditioned medium (CM) from CAPAN-1 human pancreatic tumor cells that contain Shh (FIG. 9F) (Wang et al., Cells 2019, 8, 509 and Stappenbeck et al., Cells 2019, 8, 1297) in the presence or absence of the compounds at 10 μM. Cellular mRNAs were extracted to quantify a TGFβ-downstream gene, connective tissue growth factor (CTGF) (FIG. 9E), a Hh target gene, Gli1 (FIG. 9F), and Oaz1 for normalization of CTGF and Gli1 (FIGS. 9E and 9F). (FIG. 9G) At 24 h post-inoculation, Viral RNA produced from the cells treated with DMSO, 10 μM RDV, 10 μM HPI-1, 10 μM GDC-0449, or 10 μM SB-431542, was quantified with real-time RT-PCR All data are shown with error bars indicating S.D., ** p<0.01 vs. DMSO; N.S., not significant, with Student's t-test.

FIGS. 10A-10D show Oxy210 inhibits the SARS-CoV-2 genome replication. (FIG. 10A) Determination of the target step of compounds in the SARS-CoV-2 life cycle using time of additional analysis. The left diagram shows the life cycle of SARS-CoV-2, including the steps for viral entry, replication, and release. The upper right diagram shows the experimental procedures of the time of additional analysis. The assay was performed under three different conditions (a, whole; b, entry; c, post-entry): (a) the cells were treated with the compounds for 24 h throughout the whole procedure (whole life cycle); (b) compounds were added during the 1 h virus inoculation and then removed after an additional 2 h treatment (entry); (c) compounds were added at 2 h post-inoculation and presented for the remaining 21 h until harvest (post-entry). Solid and dashed boxes indicate the periods of presence and absence of the compounds, respectively. The lower right graph shows the real-time RT-PCR quantified viral RNA produced from the cells treated with 15 μM RDV, 15 μM CLQ, 10 μM Oxy210, or 10 μM Oxy133 under the three experimental conditions. All data are shown with error bars indicating S.D., * p<0.05 vs. DMSO; ** p<0.01 vs. DMSO; N.S., not significant; with Student's t-test. (FIG. 10B) SARS-CoV-2 infected (panel b,c) or uninfected (panel a) cells were treated with the compounds (b, DMSO; c, 10 μM Oxy210) as indicated and examined with electron microscopy. Images in panels d-f show the insets in panels a-c, respectively, at higher magnification. N, nucleus; M, mitochondria; *, double-membrane vesicle. (FIG. 10C) Hepatitis C virus (HCV) replication was evaluated by measuring the luciferase activity in LucNeo #2 cells carrying the discistronic HCV NN (genotype-1b) subgenomic replicon RNA and the luciferase gene (see Materials and Methods), treated with or without DMSO, 10 μM Oxy210, or 1 μM sofosbuvir as a positive control for 48 h. (FIG. 10D) Hepatitis D virus (HDV) replication was measured by quantifying HDV RNA using real-time RT-PCR in HepG2-hNTCP-C4 cells infected with HDV and treated with or without DMSO or 10 μM Oxy210 for six days. 200 nM myrcludex-B (MyrB) was used as a positive control to inhibit HDV infection.

FIG. 11 shows pharmacokinetics of Oxy210 in Mice.

FIGS. 12A-12C depict dose-response curves for Oxy232. (FIG. 12A) Secreted viral RNA in the VeroE6 culture supernatant 24 h post-inoculation was quantified by real-time RT-PCR. Cell viability of Oxy232 treated cells evaluated with an MTT assay. (FIG. 12B) Secreted viral RNA of 3 SARS-CoV-2 strains in the VeroE6 culture supernatant 24 h post-inoculation was quantified by real-time RT-PCR. (FIG. 12C) Calu-3 (ATCC) cells were pretreated with Oxy232 for 2 hours prior to continuous infection with SARS-CoV-2 (isolate USA WA1/2020) at MOI=0.5. 48 h post-infection, cells were fixed, immune-strained, and imaged by automated microscopy for infection (dsRNA+ cells/total cell number) and cell number.

FIG. 13 depicts lung exposure of Oxy232 in mice. Oral dosing at 200 mg/kg, formulated in 10% dimethylsulfoxide (DMSO), 10% ethanol, 5% polyethylene glycol (PEG) 400/5% corn oil (20 mg/ml). The dosing volume was 10 mL/kg.

FIG. 14 are electron microscopy photographs. Uninfected cells or cells infected with SARS-CoV-2 and treated with DMSO, RDV (15 μM) or Oxy232 (10 μM) were examined by electron microscopy. Images show insets at higher magnification.

FIG. 15A is a schematic representation of an assay for determining effect of the compounds on SARS-CoV-2 propagation. VeroE6/TMPRSS2 cells were inoculated with SARS-CoV-2 for 1 h. After wash out free viruses, cells were cultured with compounds (remdesivir 20 uM, 7-ketocholesterol 40 uM, and 27-hydroxycholesterol 20 uM) for 48 h and were observed with microscopy.

FIG. 15B shows effect of endogenous oxysterols on SARS-CoV-2 propagation. 7-ketocholesterol and 27-hydroxycholesterol as well as remdesivir inhibited SARS-CoV-2-induced cytopathic effect (CPE).

FIG. 16A is a schematic representation of the assay for determining the anti-SARS-CoV-2 effect of the compounds of Formula (I). VeroE6/TMPRSS2 cells were infected with SARS-CoV-2 for 1 h and treated with oxysterol derivatives at 10 or 30 uM. Viral RNA in the culture supernatant was quantified by real time RT-PCR analysis at 24 h post-inoculation.

FIG. 16B shows the anti-SARS-CoV-2 effect of some exemplary compounds of Formula (I). Several oxysterols such as oxy210, oxy232, oxy233, and oxy243 strongly reduced viral RNA levels.

FIG. 17A is a schematic representation of an assay for determining effect of the compounds on SARS-CoV-2 propagation. VeroE6/TMPRSS2 cells were infected with SARS-CoV-2 and treated with compounds (remdesivir 10 uM, oxy8 7.5 uM, oxy16 20 uM, oxy186 20 uM, and oxy210 10 uM) as shown in FIG. 15A. After 48 h, SARS-CoV-2-induced cytopathic effect was observed by microscopy.

FIG. 17B shows exemplary compounds oxy8, oxy16, oxy186, and oxy210 protected cells from cytopathic effect.

FIG. 18A is a schematic representation of an assay for measuring the dose response for the antiviral activity of the compounds. VeroE6/TMPRSS2 cells were inoculated with SARS-CoV-2 for 1 h. After wash out free viruses, cells were cultured with compounds at various concentrations. Viral RNA produced from infected cells into the culture supernatant was quantified by real time RT-PCR analysis.

FIG. 18B shows exemplary compound Oxy210 showed highly potent antiviral effect against SARS-CoV-2.

FIGS. 19A-19D show structures of some exemplary compounds.

FIG. 20 depicts structures of some natural oxysterols.

### DETAILED DESCRIPTION

Specific inhibition of virus entry into the cell is an attractive therapeutic concept to control and eventually eliminate acute and chronic infections by different viruses. Entry inhibition has curative potential. For achieving efficient entry into cells, viruses utilize multiple host factors for mediating the stepwise entry process. For Example, sodium taurocholate cotransporting polypeptide (NTCP) is a hepatitis B virus (HBV) preS1-specific receptor which plays a key role in Hepatitis B virus (HBV) and/or Hepatitis D virus (HDV) infection. NTCP is a sodium-dependent transporter for taurocholic acid. NTCP is expressed at the basolateral membrane of hepatocytes and mediates the transport of conjugated bile acids and some drugs from blood to hepatocytes NTCP specifically interacts with the preS1 region of the large surface protein of HBV, thereby functioning as a viral entry receptor.

The compounds of Formula (I) can be used in methods for inhibiting virus entry into a cell. As a consequence, compounds of Formula (I) can be expected to exhibit a very broad spectrum of activity, covering viruses of all classes, regardless of their genome composition (RNA vs DNA viruses). Accordingly, in some embodiments of any one of the aspects, the viral infection is by a virus from a virus family selected from the group consisting of abyssoviridae, ackermannviridae, adenoviridae, alloherpesviridae, alphaflexiviridae, alphasatellitidae, alphatetraviridae, alvernaviridae, amalgaviridae, amnoonviridae, ampullaviridae, anelloviridae, arenaviridae, arteriviridae, artoviridae, ascoviridae, asfarviridae, aspiviridae, astroviridae, autographiviridae, avsunviroidae, bacilladnaviridae, baculoviridae, barnaviridae, belpaoviridae, benyviridae, betaflexivridae, bicaudaviridae, bidnavwndae, birnaviridae, bornaviridae, botourmiaviridae, bmmoviridae, caliciviridae, carmotetraviridae, caulimoviridae, chaseviridae, chrysoviridae, chuviridae, circoviridae, clavaviridae, closteroviridae, comnaviridae, corticoviridae, cremegaviridae, cruliviridae, cystoviridae, deltaJlexiviridae, demerecviridae, dicistroviridae, drexlerviridae, endornaviridae, euroniviridae, filoviridae, fimoviridae, finnlakeviridae, flavwvridae, fusellovridae, gammaflexiviridae, geminiviridae, genomoviridae, globuloviridae, gresnaviridae, guttavrndae, halspiviridae, hantavwndae, hepadnaviridae, hepeviridae, herelleviridae, herpesviridae, hypoviridae, hytrosaviridae, flaviridae, inoviridae, iridoviridae, kiaviridae, lavidaviridae, leishbuviridae, leviviridae, lipothrixviridae, lispiviridae, luteoviridae, malacoherpesviridae, marnaviridae, marseilleviridae, matonaviridae, mayoviridae, medionivridae, megabirnaviridae, mesonivridae, metaviridae, microvndae, mimivridae, mitoviridae, mononiviridae, mymonaviridae, myoviridae, mypoviridae, nairoviridae, nanghoshaviridae, nanhypoviridae, nanoviridae, namaviridae, nimaviridae, nodaviridae, nudiviridae, nyamiviridae, olifoviridae, orthomyxoviridae, ovaliviridae, papillomaviridae, paramyxoviridae, partitiviridae, parvoviridae, peribunyaviridae, permutotetravindae, phasmaviridae, phenuiviridae, phycodnaviridae, picobirnaviridae, picornaviridae, plasmaviridae, plectroviridae, pleolipoviridae, pneumoviridae, podoviridae, polycipiviridae, polydnaviridae, polymycoviridae, polyomaviridae, portogloboviridae, pospiviroidae, potyviridae, poxviridae, pseudoviridae, qinviridae, quadriviridae, redondoviridae, reoviridae, retroviridae, rhabdoviridae, roniviridae, rudiviridae, sarthroviridae, secoviridae, sinhaliviridae, siphoviridae, smacoviridae, solemoviridae, solinviviridae, sphaerolipoviridae, spiraviridae, sunviridae, tectiviridae, thaspiviridae, tobanivridae, togaviridae, tolecusatellitidae, tombusviridae, tospoviridae, totiviridae, tristromaviridae, turriviridae, tymoviridae, virgaviridae, wupedeviridae, xinmoviridae, and yueviridae.

In some embodiments of any one of the aspects, the viral infection is by a virus selected from the group consisting of hepadnaviruses, coronaviruses, avian influenza viruses, adenoviruses, herpesviruses, human papillomaviruses, parvoviruses, reoviruses, picornaviruses, flaviviruses, togaviruses, orthomyxovirus, bunyaviruses, rhabdoviruses, and paramyxoviruses.

In some embodiments of any one of the aspects, the viral infection is caused by a virus selected from the group consisting of adeno-associated virus; Aichi virus; astrovirus; Australian bat lyssavirus; BK polyomavirus; Banna virus; Barmah forest virus; Bunyamwera virus; Bunyavirus La Crosse; Bunyavirus snowshoe hare; Cercopithecine herpesvirus; Chandipura virus; Chikungunya virus; Cosavirus A; Cowpox virus; Coxsackie A virus; Coxsackie B virus; Crimean-Congo hemorrhagic fever virus; Dengue virus; Dhori virus; Dugbe virus; Duvenhage virus; Eastern equine encephalitis virus; Ebolavirus; Echovirus; Encephalomyocarditis virus; Epstein-Barr virus; European bat lyssavirus; GB virus C/Hepatitis G virus; Hantaan virus; Hendra virus; Hepatitis A virus; Hepatitis B virus; Hepatitis C virus; Hepatitis D virus; Hepatitis E virus; Hepatitis delta virus; Horsepox virus; Human adenovirus; Human astrovirus; Human coronavirus; Human cytomegalovirus; Human enterovirus 68, 70; Human herpesvirus 1; Human herpesvirus 2; Human herpesvirus 6; Human herpesvirus 7; Human herpesvirus 8; Human immunodeficiency virus; Human papillomavirus 1; Human papillomavirus 2; Human papillomavirus 16,18; Human parainfluenza; Human parvovirus B19; Human respiratory syncytial virus; Human rhinovirus; Human SARS coronavirus; Human spumaretrovirus; Human T-lymphotropic virus; Human torovirus; Influenza A virus; Influenza B virus; Influenza C virus; Isfahan virus; JC polyomavirus; Japanese encephalitis virus; Junin arenavirus; KI Polyomavirus; Kunjin virus; Lagos bat virus; Lake Victoria marburgvirus; Langat virus; Lassa virus; Lordsdale virus; Louping ill virus; Lymphocytic choriomeningitis virus; Machupo virus; Mayaro virus; MERS coronavirus; Measles virus; Mengo encephalomyocarditis virus; Merkel cell polyomavirus; Mokola virus; Molluscum contagiosum virus; Monkeypox virus; Mumps virus; Murray valley encephalitis virus; New York virus; Nipah virus; norovirus; Norwalk virus; O'nyong-nyong virus; Orf virus; Oropouche virus; Pichinde virus; Poliovirus; Punta toro phlebovirus; Puumala virus; Rabies virus; Rift valley fever virus; Rosavirus A; Ross river virus; Rotavirus A; Rotavirus B; Rotavirus C; Rubella virus; Sagiyama virus; Salivirus A; Sandfly fever sicilian virus; Sapporo virus; SARS coronavirus 2; Semliki forest virus; Seoul virus; Simian foamy virus; Simian virus 5; Sindbis virus; Southampton virus; St. louis encephalitis virus; Tick-borne powassan virus; Torque teno virus; Toscana virus; Uukuniemi virus; Vaccinia virus; Varicella-zoster virus; Variola virus; Venezuelan equine encephalitis virus; Vesicular stomatitis virus; Western equine encephalitis virus; WU polyomavirus; West Nile virus; Yaba monkey tumor virus; Yaba-like disease virus; Yellow fever virus; and Zika virus.

Some preferred viral infections include liver infections such as hepatitis, and respiratory infections of the nose, throat, upper airways, and lungs such as influenza, pneumonia, coronavirus, SARS coronavirus, SARS-CoV-2 virus, bronchoiolitis, and laryngotracheobronchitis.

It is noted that the viral infection can be any where in the subject. For example, the viral infection can be an infection of a tissue selected from the group consisting of: liver tissue, upper respiratory system tissue, lower respiratory system tissue, lung tissue, central nervous system tissue, eye tissue, kidney tissue, bladder tissue, spleen tissue, cardiac tissue, gastrointestinal tissue, epidermal tissue, reproductive tissue, nasal cavity tissue, larynx tissue, trachea tissue, bronchi tissue, oral cavity tissue, blood tissue, and muscle tissue.

In some embodiments of any one of the aspects, the viral infection is an infection of the liver. For example, the viral infection is a hepatitis infection, such as a hepatitis A, B, C, D or E infection. In some embodiments of any one of the aspects, the viral infection is a hepatitis B (HBV) or hepatitis D (HDV) infection.

In some embodiments of any one of the aspects, the viral infection is an infection of respiratory system. For example, the viral infection is a coronavirus infection. The coronavirus can be selected from the group consisting of: severe acute respiratory syndrome-associated coronavirus (SARS-CoV); severe acute respiratory syndrome-associated coronavirus 2 (SARS-CoV-2); Middle East respiratory syndrome-related coronavirus (MERS-CoV); HCoV-NL63, and HCoV-HKu1. In some embodiments of any one of the aspects, the coronavirus is SARS-CoV-2.

In some embodiments, the viral infection is a human immunodeficiency virus (HIV) infection.

It is noted that the virus can be a DNA virus or an RNA virus. Further, when the virus is an RNA virus, it can be a positive strand RNA virus or a negative strand RNA virus.

Risk factors for having or developing a viral infection include exposure to the virus, exposure or contact with a subject infected with a virus, exposure to contaminated surfaces contacted with a virus, contact with a biological sample or bodily fluid from a subject infected by a virus, sexual intercourse with a subject infected by a virus, needle sharing, blood transfusions, drug use, and any other risk factor known in the art to transmit a virus from one subject to another. Risk factors for a subject can be evaluated, e.g., by a skilled clinician or by the subject.

### Combination Therapy

In some embodiments of any one of the aspects, a method described herein further comprises administering, e.g., co-administering, at least one additional therapeutic the subject.

In some embodiments of any one of the aspects, the additional therapeutic is an anti-viral therapeutic. Exemplary anti-viral therapeutics include, but are not limited to, Abacavir, Acyclovir (Aciclovir), Adefovir, Amantadine, Ampligen, Amprenavir (Agenerase), Arbidol, Atazanavir, Atripla, Balavir, Baloxavir marboxil (Xofluza®), Biktarvy Boceprevir (Victrelis®), Cidofovir, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Fomivirsen, Fosamprenavir, Foscamet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Lamivudine, Letermovir (Prevymis®), Lopinavir, Loviride, Maraviroc, Methisazone, Moroxydine, Nelfinavir, Nevirapine, Nexavir®, Nitazoxanide, Norvir, Nucleoside analogues, Oseltamivir (Tamiflu®), Peginterferon alfa-2a, Peginterferon alfa-2b, Penciclovir, Peramivir (Rapivab®), Pleconaril, Podophyllotoxin, Protease inhibitor (pharmacology), Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Telaprevir, Telbivudine (Tyzeka®), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza®), and Zidovudine.

In some embodiments of any one of the aspects, the additional therapeutic is an immunostimulatory agent.

For treating HBV infections, additional therapeutic can be selected from the group consisting of HBV DNA polymerase inhibitors, toll-like receptor 7 modulators, toll-like receptor 8 modulators, Toll-like receptor 7 and 8 modulators, Toll-like receptor 3 modulators, interferon alpha ligands, HBsAg inhibitors, compounds targeting HbcAg, cyclophilin inhibitors, HBV therapeutic vaccines, HBV prophylactic vaccines, HBV viral entry inhibitors, NTCP inhibitors or binders, receptor tyrosine kinase inhibitors, antisense oligonucleotide targeting viral mRNA, short interfering RNAs (siRNA), hepatitis B virus E antigen inhibitors, HBx inhibitors, cccDNA inhibitors, HBV transcription inhibitors, HBV RNA destabilizers, RNaseH inhibitors, HBV antibodies including HBV antibodies targeting the surface antigens of the hepatitis B virus, nucleic acid polymers, thymosin agonists, cytokines, HBV core or capsid protein inhibitors, stimulators of retinoic acid-inducible gene 1, stimulators of NOD2, recombinant thymosin alpha-1 and hepatitis B virus replication inhibitors, and combinations thereof. For example, the additional therapeutic can be selected from the group consisting of HBV combination drugs, other drugs for treating HBV, 3-dioxygenase (IDO) inhibitors, antisense oligonucleotide targeting viral mRNA, Apolipoprotein A1 modulator, arginase inhibitors, B- and T-lymphocyte attenuator inhibitors, Bruton's tyrosine kinase (BTK) inhibitors, CCR2 chemokine antagonist, CD137 inhibitors, CD160 inhibitors, CD305 inhibitors, CD4 agonist and modulator, compounds targeting HBcAg, compounds targeting hepatitis B core antigen (HBcAg), covalently closed circular DNA (cccDNA) inhibitors, cyclophilin inhibitors, cytokines, cytotoxic T-lymphocyte-associated protein 4 (ipi4) inhibitors, DNA polymerase inhibitor, Endonuclease modulator, epigenetic modifiers, Farnesoid X receptor agonist, gene modifiers or editors, HBsAg inhibitors, HBsAg secretion or assembly inhibitors, HBV antibodies, HBV DNA polymerase inhibitors, HBV replication inhibitors, HBV RNAse inhibitors, HBV vaccines, HBV viral entry inhibitors. HBx inhibitors, Hepatitis B large envelope protein modulator, Hepatitis B large envelope protein stimulator, Hepatitis B structural protein modulator, hepatitis B surface antigen (HBsAg) inhibitors, hepatitis B surface antigen (HBsAg) secretion or assembly inhibitors, hepatitis B virus E antigen inhibitors, hepatitis B virus replication inhibitors, Hepatitis virus structural protein inhibitor, HIV-1 reverse transcriptase inhibitor, Hyaluronidase inhibitor, IAPs inhibitors, TL-2 agonist, TL-7 agonist, Immunoglobulin agonist, Immunoglobulin G modulator, immunomodulators, indoleamine-2, inhibitors of ribonucleotide reductase, Interferon agonist, Interferon alpha 1 ligand, Interferon alpha 2 ligand, Interferon alpha 5 ligand modulator, Interferon alpha ligand, Interferon alpha ligand modulator, interferon alpha receptor ligands, Interferon beta ligand. Interferon ligand, Interferon receptor modulator, Interleukin-2 ligand, ipi4 inhibitors, lysine demethylase inhibitors, histone demethylase inhibitors, KDM5 inhibitors, KDMI inhibitors, killer cell lectin-like receptor subfamily G member 1 inhibitors, lymphocyte-activation gene 3 inhibitors, lymphotoxin beta receptor activators, microRNA (miRNA) gene therapy agents, modulators of Axl, modulators of B7-H3, modulators of B7-H4, modulators of CD160, modulators of CD161, modulators of CD27, modulators of CD47, modulators of CD70, modulators of GITR, modulators of HEVEM, modulators of ICOS, modulators of Mer, modulators of NKG2A, modulators of NKG2D, modulators of OX40, modulators of STRPalpha, modulators of TIGIT, modulators of Tim-4, modulators of Tyro, Na+-taurocholate cotransportmg polypeptide (NTCP) inhibitors, natural killer cell receptor 2B4 inhibitors, NOD2 gene stimulator, Nucleoprotein inhibitor, nucleoprotein modulators, PD-1 inhibitors, PD-L1 inhibitors, PEG-Tnterferon Lambda, Peptidylprolyl isomerase inhibitor, phosphatidylinositol-3 kinase (PI3K) inhibitors, recombinant scavenger receptor A (SRA) proteins, recombinant thymosin alpha-1, Retinoic acid-inducible gene 1 stimulator, Reverse transcriptase inhibitor, Ribonuclease inhibitor, RNA DNA polymerase inhibitor, short interfering RNAs (siRNA), short synthetic hairpin RNAs (sshRNAs), SLC10A1 gene inhibitor, SMAC mimetics, Src tyrosine kinase inhibitor, stimulator of interferon gene (STING) agonists, stimulators of NOD1, T cell surface glycoprotein CD28 inhibitor, T-cell surface glycoprotein CD8 modulator, Thymosin agonist, Thymosin alpha 1 ligand, Tim-3 inhibitors, TLR-3 agonist, TLR-7 agonist, TLR-9 agonist, TLR9 gene stimulator, toll-like receptor (TLR) modulators, Viral ribonucleotide reductase inhibitor, zinc finger nucleases or synthetic nucleases (TALENs), and combinations thereof.

The compounds of Formula (I) can inhibit virus entry into cells. Accordingly, a compound of Formula (I), or a pharmaceutically acceptable salt or solvate thereof, can be administered to a cell for inhibiting virus entry.

It is noted that administering to the cell can be in vitro or in-vivo. Methods for administering a compound to a cell are well known and available to one of skill in the art. As used herein, administering the compound to the cell means contacting the cell with the compound so that the compound is taken up by the cell. Generally, the cell can be contacted with the compound in a cell culture e.g., in vitro or ex vivo, or the compound can be administrated to a subject, e.g., in vivo. The term “contacting” or “contact” as used herein in connection with contacting a cell includes subjecting the cells to an appropriate culture media, which comprises a compound of Formula (I). Where the cell is in vivo, “contacting” or “contact” includes administering the compound, e.g., in a pharmaceutical composition to a subject via an appropriate administration route such that the compound contacts the cell in vivo.

For example, when the cell is in vitro, said administering to the cell can include subjecting the cell to an appropriate culture media which comprises the indicated compound. Where the cell is in vivo, said administering to the cell includes administering the compound to a subject via an appropriate administration route such that the compound is administered to the cell in vivo.

The cell to be administered a compound of Formula (I) can be any desired cell. For example, the cell can be a cell susceptible to infection or replication by a virus. The term “susceptible cell” as used herein refers to any cell that may be infected with a virus. One skilled in the art will readily recognize the variety of cells capable of being infected with a virus. Exemplary susceptible cells include, but are not limited to, liver or hepatic cells, primary cells, hepatoma cells, kidney cells, CaCo2 cells, dendritic cells, placental cells, endometrial cells, lymph node cells, lymphoid cells (B and T cells), peripheral blood mononuclear cells, monocytes/macrophages, epithelial cells, mesenchymal cells, and endothelial cells.

In some embodiments of any one of the aspects, the cell is a liver cell. There are four basic cell types in the liver: hepatocytes; stellate fat storing cells; Kupffer cells; and liver endothelial cells. Hepatocytes are particularly susceptible to viral infection. Accordingly, in some embodiments of any one of the aspects, the compound of Formula (I) is adminstered to a hepatocyte. In some embodiments of any one of the aspects, the cell is a Calu-3.

In some embodiments of any one of the aspects, the cell is a cell of the respiratory system. For example, the compound of Formula (I) is administered to a ciliated cells, basal cells, epithelial cells, goblet cells and/or an alveolar cells. In some embodiments of any one of the aspects, the cell is a Calu-3 cell.

### Compounds

In compounds of Formula (I), [symbol] is a single or double bond; R₁ and R₁′ are independently hydrogen, substituted or unsubstituted C₁-C₈ alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl, provided that one of R₁ and R₁′ is OH, or R₁ and R₁′ together are ═O; R₂, R₃, R₄, and R₅ are independently hydrogen, deuterium, C₁-C₈alkyl, or —OH, or one of R₂ or R₃ together with one of R₄ or R₅ forms a double bond; R₆ is alkyl, aryl or heteroaryl, wherein the alkyl, aryl or the heteroaryl are optionally substituted with 1, 2, 3, or 4 R₉ groups; R₇ is hydrogen, substituted or unsubstituted C₁-C₈alkyl, or —C(O)NR₁₀R₁₁; R₈ is hydrogen or —OH; each R₉ is independently selected from deuterium, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₂₋₉heteroaryl, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR₁₂, —SR₁₂, —N(R₁₃)(R₁₃), —C(O)OR₁₃, —C(O)N(R₁₃)(R₁₄), —C(O)R₁₅, —S(O)₂R₁₅, and —S(O)₂N(R₁₃)(R₁₄); R₁₀ and R₁₁ are independently hydrogen, substituted or unsubstituted C₁-C₈alkyl, or substituted or unsubstituted aryl; each R₁₂ is independently selected from H, C₁₋₆alkyl, C₁₋₆haloalkyl, C₃₋₆cycloalkyl, C₂₋₉heterocycloalkyl, C₆₋₁₀aryl, and C₁₋₆heteroaryl; each R₁₃ and each R₁₄ are each independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl; and each R₁₅ is independently selected from C₁₋₆alkyl, C₁₋₆haloalkyl, C₃₋₆cycloalkyl, C₂₋₉heterocycloalkyl, C₆₋₁₀aryl, and C₁₋₉heteroaryl.

In some embodiments of the any one of the aspects described herein, [symbol] is a single. In some other embodiments of any one of the aspects described herein, [symbol] is a double bond.

In some embodiments of any one of the aspects described herein, R₁ is substituted or unsubstituted C₁-C₈alkyl. In some embodiments of any one of the aspects described herein, R₁ is substituted C₁-C₈alkyl. In some embodiments of any one of the aspects described herein, R₁ is —CF₃. In some embodiments of any one of the aspects described herein, R₁ is unsubstituted C₁-C₈alkyl. In some embodiments of any one of the aspects described herein, R₁ is unsubstituted C₁-C₄alkyl. In some embodiments of any one of the aspects described herein, R₁ is —CH₃. In some embodiments of any one of the aspects described herein, R₁ is —CH₂CH₃. In some embodiments of any one of the aspects described herein, R₁ is substituted or unsubstituted aryl. In some embodiments of any one of the aspects described herein, R₁ is unsubstituted phenyl. In some embodiments of any one of the aspects described herein, R₁ is substituted or unsubstituted C₁-C₈alkyl or substituted or unsubstituted phenyl. In some embodiments of any one of the aspects described herein, R₁ is H. In some embodiments of any one of the aspects described herein, R₁ is OH.

In some embodiments of any one of the aspects described herein, R₁′ is substituted or unsubstituted C₁-C₈alkyl. In some embodiments of any one of the aspects described herein, R₁′ is substituted C₁-C₈alkyl. In some embodiments of any one of the aspects described herein, R₁′ is —CF₃. In some embodiments of any one of the aspects described herein, R₁′ is unsubstituted C₁-C₈alkyl. In some embodiments of any one of the aspects described herein, R₁′ is unsubstituted C₁-C₄alkyl. In some embodiments of any one of the aspects described herein, R₁′ is —CH₃. In some embodiments of any one of the aspects described herein, R₁′ is —CH₂CH₃. In some embodiments of any one of the aspects described herein, R₁′ is substituted or unsubstituted aryl. In some embodiments of any one of the aspects described herein, R₁′ is unsubstituted phenyl. In some embodiments of any one of the aspects described herein, R₁′ is substituted or unsubstituted C₁-C₈alkyl or substituted or unsubstituted phenyl. In some embodiments of any one of the aspects described herein, R₁′ is H. In some embodiments of any one of the aspects described herein, R₁′ is OH.

In some embodiments of any one of the aspects described herein, one of R₁ and R₁′ is OH, e.g., only one of R₁ and R₁′ is OH. Accordingly, in some embodiments, R₁′ is OH. In some other embodiments, R₁ is OH.

In some embodiments of any one of the aspects described herein, R₁′ is OH and R₁ is substituted or unsubstituted C₁-C₈alkyl. In some embodiments of any one of the aspects described herein, R₁′ is OH and R₁ is substituted C₁-C₈alkyl. In some embodiments of any one of the aspects described herein, R₁′ is OH and R₁ is —CF₃. In some embodiments of any one of the aspects described herein, R₁′ is OH and R₁ is unsubstituted C₁-C₄alkyl. In some embodiments of any one of the aspects described herein, R₁′ is OH and R₁ is unsubstituted C₁-C₄alkyl. In some embodiments of any one of the aspects described herein, R₁′ is OH and R₁ is —CH₃. In some embodiments of any one of the aspects described herein, R₁′ is OH and R₁ is —CH₂CH₃. In some embodiments of any one of the aspects described herein, R₁′ is OH and R₁ is substituted or unsubstituted aryl. In some embodiments of any one of the aspects described herein, R₁′ is OH and R₁ is unsubstituted phenyl. In some embodiments of any one of the aspects described herein, R₁′ is OH and R₁ is or unsubstituted C₁-C₈alkyl or substituted or unsubstituted phenyl. In some embodiments of any one of the aspects described herein, R₁′ is OH and R₁ is H.

In some embodiments of any one of the aspects described herein, R₁ is OH and R₁′ is substituted or unsubstituted C₁-C₈alkyl. In some embodiments of any one of the aspects described herein, R₁ is OH and R₁′ is substituted C₁-C₈alkyl. In some embodiments of any one of the aspects described herein, R₁ is OH and R₁′ is —CF₃. In some embodiments of any one of the aspects described herein, R₁ is OH and R₁′ is unsubstituted C₁-C₈alkyl. In some embodiments of any one of the aspects described herein, R₁ is OH and R₁′ is unsubstituted C₁-C₄alkyl. In some embodiments of any one of the aspects described herein, R₁ is OH and R₁′ is —CH₃. In some embodiments of any one of the aspects described herein, R₁ is OH and R₁′ is —CH₂CH₃. In some embodiments of any one of the aspects described herein, R₁ is OH and R₁′ is substituted or unsubstituted aryl. In some embodiments of any one of the aspects described herein, R₁ is OH and R₁′ is unsubstituted phenyl. In some embodiments of any one of the aspects described herein, R₁ is OH and R₁′ is substituted or unsubstituted C₁-C₈alkyl or substituted or unsubstituted phenyl. In some embodiments of any one of the aspects described herein, R₁ is OH and R₁′ is H.

In some embodiments of any one of the aspects described herein, R₁ and R₁′ together are ═O.

In compounds of any one of the aspects, R₂ can be hydrogen, deuterium, C₁-C₈alkyl, or —OH. For example, R₂ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is hydrogen, deuterium, or —OH. In some embodiments, R₂ is hydrogen. In some other embodiments, R₂ is deuterium. In still some other embodiments, R₂ is OH.

In compounds of any one of the aspects, R₃ can be hydrogen, deuterium, C₁-C₈alkyl, or —OH. For example, R₃ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is hydrogen, deuterium, or —OH. In some embodiments, R₃ is hydrogen. In some other embodiments, R₃ is deuterium. In still some other embodiments, R₃ is OH.

In compounds of any one of the aspects, R₄ can be hydrogen, deuterium, C₁-C₈alkyl, or —OH. For example, R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₄ is hydrogen, deuterium, or —OH. In some embodiments, R₄ is hydrogen. In some other embodiments, R₄ is deuterium. In still some other embodiments, R₄ is OH.

In compounds of any one of the aspects, R₅ can be hydrogen, deuterium, C₁-C₈alkyl, or —OH. For example, R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₅ is hydrogen, deuterium, or —OH. In some embodiments, R₅ is hydrogen. In some other embodiments, R₅ is deuterium. In still some other embodiments, R₅ is OH.

In compounds of any one of the aspects, R₂ is hydrogen and R₃ can be hydrogen, deuterium, C₁-C₈alkyl, or —OH. For example, R₂ is hydrogen and R₃ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is hydrogen and R₃ is hydrogen, deuterium, or —OH. In some embodiments, R₂ is hydrogen and R₃ is hydrogen. In some other embodiments, R₂ is hydrogen and R₃ is deuterium. In still some other embodiments, R₂ is hydrogen and R₃ is OH.

In compounds of any one of the aspects, R₂ is deuterium and R₃ can be hydrogen, deuterium, C₁-C₈alkyl, or —OH. For example, R₂ is deuterium and R₃ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is deuterium and R₃ is hydrogen, deuterium, or —OH. In some embodiments, R₂ is deuterium and R₃ is hydrogen. In some other embodiments, R₂ is deuterium and R₃ is deuterium. In still some other embodiments, R₂ is deuterium and R₃ is OH.

In compounds of any one of the aspects, R₂ is C₁-C₈alkyl and R₃ can be hydrogen, deuterium, C₁-C₈alkyl, or —OH. For example, R₂ is C₁-C₈alkyl and R₃ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is C₁-C₈alkyl and R₃ is hydrogen, deuterium, or —OH. In some embodiments, R₂ is C₁-C₈alkyl and R₃ is hydrogen. In some other embodiments, R₂ is C₁-C₈alkyl and R₃ is deuterium. In still some other embodiments, R₂ is C₁-C₈alkyl and R₃ is OH.

In compounds of any one of the aspects, R₂ is OH and R₃ can be hydrogen, deuterium, C₁-C₈alkyl, or —OH. For example, R₂ is OH and R₃ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is OH and R₃ is hydrogen, deuterium, or —OH. In some embodiments, R₂ is OH and R₃ is hydrogen. In some other embodiments, R₂ is OH and R₃ is deuterium. In still some other embodiments, R₂ is OH and R₃ is OH.

In compounds of any one of the aspects, R₂ is hydrogen and R₄ can be hydrogen, deuterium, C₁-C₈alkyl, or —OH. For example, R₂ is hydrogen and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is hydrogen and R₄ is hydrogen, deuterium, or —OH. In some embodiments, R₂ is hydrogen and R₄ is hydrogen. In some other embodiments, R₂ is hydrogen and R₄ is deuterium. In still some other embodiments, R₂ is hydrogen and R₄ is OH.

In compounds of any one of the aspects, R₂ is deuterium and R₄ can be hydrogen, deuterium, C₁-C₈alkyl, or —OH. For example, R₂ is deuterium and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is deuterium and R₄ is hydrogen, deuterium, or —OH. In some embodiments, R₂ is deuterium and R₄ is hydrogen. In some other embodiments, R₂ is deuterium and R₄ is deuterium. In still some other embodiments, R₂ is deuterium and R₄ is OH.

In compounds of any one of the aspects, R₂ is C₁-C₈alkyl and R₄ can be hydrogen, deuterium, C₁-C₈alkyl, or —OH. For example, R₂ is C₁-C₈alkyl and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is C₁-C₈alkyl and R₄ is hydrogen, deuterium, or —OH. In some embodiments, R₂ is C₁-C₈alkyl and R₄ is hydrogen. In some other embodiments, R₂ is C₁-C₈alkyl and R₄ is deuterium. In still some other embodiments, R₂ is C₁-C₈alkyl and R₄ is OH.

In compounds of any one of the aspects, R₂ is OH and R₄ can be hydrogen, deuterium, C₁-C₈alkyl, or —OH. For example, R₂ is OH and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is OH and R₄ is hydrogen, deuterium, or —OH. In some embodiments, R₂ is OH and R₄ is hydrogen. In some other embodiments, R₂ is OH and R₄ is deuterium. In still some other embodiments, R₂ is OH and R₄ is OH.

In compounds of any one of the aspects, R₂ is hydrogen and R₅ can be hydrogen, deuterium, C₁-C₈alkyl, or —OH. For example, R₂ is hydrogen and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is hydrogen and R₅ is hydrogen, deuterium, or —OH. In some embodiments, R₂ is hydrogen and R₅ is hydrogen. In some other embodiments, R₂ is hydrogen and R₅ is deuterium. In still some other embodiments, R₂ is hydrogen and R₅ is OH.

In compounds of any one of the aspects, R₂ is deuterium and R₅ can be hydrogen, deuterium, C₁-C₈alkyl, or —OH. For example, R₂ is deuterium and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is deuterium and R₅ is hydrogen, deuterium, or —OH. In some embodiments, R₂ is deuterium and R₅ is hydrogen. In some other embodiments, R₂ is deuterium and R₅ is deuterium. In still some other embodiments, R₂ is deuterium and R₅ is OH.

In compounds of any one of the aspects, R₂ is C₁-C₈alkyl and R₅ can be hydrogen, deuterium, C₁-C₈alkyl, or —OH. For example, R₂ is C₁-C₈alkyl and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is C₁-C₈alkyl and R₅ is hydrogen, deuterium, or —OH. In some embodiments, R₂ is C₁-C₈alkyl and R₅ is hydrogen. In some other embodiments, R₂ is C₁-C₈alkyl and R₅ is deuterium. In still some other embodiments, R₂ is C₁-C₈alkyl and R₅ is OH.

In compounds of any one of the aspects, R₂ is OH and R₅ can be hydrogen, deuterium, C₁-C₈alkyl, or —OH. For example, R₂ is OH and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₂ is OH and R₅ is hydrogen, deuterium, or —OH. In some embodiments, R₂ is OH and R₅ is hydrogen. In some other embodiments, R₂ is OH and R₅ is deuterium. In still some other embodiments, R₂ is OH and R₅ is OH.

In compounds of any one of the aspects, R₃ is hydrogen and R₄ can be hydrogen, deuterium, C₁-C₈alkyl, or —OH. For example, R₃ is hydrogen and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is hydrogen and R₄ is hydrogen, deuterium, or —OH. In some embodiments, R₃ is hydrogen and R₄ is hydrogen. In some other embodiments, R₃ is hydrogen and R₄ is deuterium. In still some other embodiments, R₃ is hydrogen and R₄ is OH.

In compounds of any one of the aspects, R₃ is deuterium and R₄ can be hydrogen, deuterium, C₁-C₈alkyl, or —OH. For example, R₃ is deuterium and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is deuterium and R₄ is hydrogen, deuterium, or —OH. In some embodiments, R₃ is deuterium and R₄ is hydrogen. In some other embodiments, R₃ is deuterium and R₄ is deuterium. In still some other embodiments, R₃ is deuterium and R₄ is OH.

In compounds of any one of the aspects, R₃ is C₁-C₈alkyl and R₄ can be hydrogen, deuterium, C₁-C₈alkyl, or —OH. For example, R₃ is C₁-C₈alkyl and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is C₁-C₈alkyl and R₄ is hydrogen, deuterium, or —OH. In some embodiments, R₃ is C₁-C₈alkyl and R₄ is hydrogen. In some other embodiments, R₃ is C₁-C₈alkyl and R₄ is deuterium. In still some other embodiments, R₃ is C₁-C₈alkyl and R₄ is OH.

In compounds of any one of the aspects, R₃ is OH and R₄ can be hydrogen, deuterium, C₁-C₈alkyl, or —OH. For example, R₃ is OH and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is OH and R₄ is hydrogen, deuterium, or —OH. In some embodiments, R₃ is OH and R₄ is hydrogen. In some other embodiments, R₃ is OH and R₄ is deuterium. In still some other embodiments, R₃ is OH and R₄ is OH.

In compounds of any one of the aspects, R₃ is hydrogen and R₅ can be hydrogen, deuterium, C₁-C₈alkyl, or —OH. For example, R₃ is hydrogen and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is hydrogen and R₅ is hydrogen, deuterium, or —OH. In some embodiments, R₃ is hydrogen and R₅ is hydrogen. In some other embodiments, R₃ is hydrogen and R₅ is deuterium. In still some other embodiments, R₃ is hydrogen and R₅ is OH.

In compounds of any one of the aspects, R₃ is deuterium and R₅ can be hydrogen, deuterium, C₁-C₈alkyl, or —OH. For example, R₃ is deuterium and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is deuterium and R₅ is hydrogen, deuterium, or —OH. In some embodiments, R₃ is deuterium and R₅ is hydrogen. In some other embodiments, R₃ is deuterium and R₅ is deuterium. In still some other embodiments, R₃ is deuterium and R₅ is OH.

In compounds of any one of the aspects, R₃ is C₁-C₈alkyl and R₅ can be hydrogen, deuterium, C₁-C₈alkyl, or —OH. For example, R₃ is C₁-C₈alkyl and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is C₁-C₈alkyl and R₅ is hydrogen, deuterium, or —OH. In some embodiments, R₃ is C₁-C₈alkyl and R₅ is hydrogen. In some other embodiments, R₃ is C₁-C₈alkyl and R₅ is deuterium. In still some other embodiments, R₃ is C₁-C₈alkyl and R₅ is OH.

In compounds of any one of the aspects, R₃ is OH and R₅ can be hydrogen, deuterium, C₁-C₈alkyl, or —OH. For example, R₃ is OH and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₃ is OH and R₅ is hydrogen, deuterium, or —OH. In some embodiments, R₃ is OH and R₅ is hydrogen. In some other embodiments, R₃ is OH and R₅ is deuterium. In still some other embodiments, R₃ is OH and R₅ is OH.

In compounds of any one of the aspects, R₄ is hydrogen and R₅ can be hydrogen, deuterium, C₁-C₈alkyl, or —OH. For example, R₄ is hydrogen and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₄ is hydrogen and R₅ is hydrogen, deuterium, or —OH. In some embodiments, R₄ is hydrogen and R₅ is hydrogen. In some other embodiments, R₄ is hydrogen and R₅ is deuterium. In still some other embodiments, R₄ is hydrogen and R₅ is OH.

In compounds of any one of the aspects, R₄ is deuterium and R₅ can be hydrogen, deuterium, C₁-C₈alkyl, or —OH. For example, R₄ is deuterium and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₄ is deuterium and R₅ is hydrogen, deuterium, or —OH. In some embodiments, R₄ is deuterium and R₅ is hydrogen. In some other embodiments, R₄ is deuterium and R₅ is deuterium. In still some other embodiments, R₄ is deuterium and R₅ is OH.

In compounds of any one of the aspects, R₄ is C₁-C₈alkyl and R₅ can be hydrogen, deuterium, C₁-C₈alkyl, or —OH. For example, R₄ is C₁-C₈alkyl and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₄ is C₁-C₈alkyl and R₅ is hydrogen, deuterium, or —OH. In some embodiments, R₄ is C₁-C₈alkyl and R₅ is hydrogen. In some other embodiments, R₄ is C₁-C₈alkyl and R₅ is deuterium. In still some other embodiments, R₄ is C₁-C₈alkyl and R₅ is OH.

In compounds of any one of the aspects, R₄ is OH and R₅ can be hydrogen, deuterium, C₁-C₈alkyl, or —OH. For example, R₄ is OH and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₄ is OH and R₅ is hydrogen, deuterium, or —OH. In some embodiments, R₄ is OH and R₅ is hydrogen. In some other embodiments, R₄ is OH and R₅ is deuterium. In still some other embodiments, R₄ is OH and R₅ is OH.

In some embodiments of any one of the aspects, R₂, R₃, R₄, and R₅ are each deuterium. In some embodiments of any one of the aspects, R₂, R₃, R₄, and R₅ are each hydrogen. In some embodiments of any one of the aspects, R₂ is —OH, and R₃, R₄, and R₅ are each hydrogen. In some embodiments of any one of the aspects, R₃ is —OH, and R₂, R₄, and R₅ are each hydrogen. In some embodiments of any one of the aspects, R₂ and R₄ are each —OH, and R₃ and R₅ are each hydrogen. In some embodiments of any one of the aspects, R₂ and R₅ are each —OH, and R₃ and R₄ are each hydrogen. In some embodiments of any one of the aspects, R₃ and R₄ are each —OH, and R₂ and R₅ are each hydrogen. In some embodiments of any one of the aspects, R₃ and R₅ are each —OH, and R₂ and R₄ are each hydrogen.

In some embodiments of any one of the aspects described herein, R₂ and R₄ form a second bond between the carbon atoms they are attached to. In some compounds of the various aspects described herein, R₂ and R₅ form a second bond between the carbon atoms they are attached to.

In compounds of any one of the aspects, R₂ and R₄ form a second bond between the carbon atoms they are attached to and R₃ can be hydrogen, deuterium, C₁-C₈alkyl, or —OH. For example, R₂ and R₄ form a second bond between the carbon atoms they are attached to and R₃ can be hydrogen, deuterium, methyl, ethyl, propyl, i-propyl, n-butyl, t-butyl, n-pentyl, hexyl, heptyl, octyl, or OH. In some embodiments of any one of the aspects, R₂ and R₄ form a second bond between the carbon atoms they are attached to and R₃ is hydrogen, deuterium, or —OH. In some embodiments, R₂ and R₄ form a second bond between the carbon atoms they are attached to and R₃ is hydrogen. In some other embodiments, R₂ and R₄ form a second bond between the carbon atoms they are attached to and R₃ is deuterium. In still some other embodiments, R₂ and R₄ form a second bond between the carbon atoms they are attached to and R₃ is OH.

In compounds of any one of the aspects, R₂ and R₄ form a second bond between the carbon atoms they are attached to and R₅ can be hydrogen, deuterium, C₁-C₈alkyl, or —OH. For example, R₂ and R₄ form a second bond between the carbon atoms they are attached to and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₂ and R₄ form a second bond between the carbon atoms they are attached to and R₅ is hydrogen, deuterium, or —OH. In some embodiments, R₂ and R₄ form a second bond between the carbon atoms they are attached to and R₅ is hydrogen. In some other embodiments, R₂ and R₄ form a second bond between the carbon atoms they are attached to and R₅ is deuterium. In still some other embodiments, R₂ and R₄ form a second bond between the carbon atoms they are attached to and R₅ is OH.

In compounds of any one of the aspects, R₂ and R₅ form a second bond between the carbon atoms they are attached to and R₃ can be hydrogen, deuterium, C₁-C₈alkyl, or —OH. For example, R₂ and R₅ form a second bond between the carbon atoms they are attached to and R₃ can be hydrogen, deuterium, methyl, ethyl, propyl, i-propyl, n-butyl, t-butyl, n-pentyl, hexyl, heptyl, octyl, or OH. In some embodiments of any one of the aspects, R₂ and R₅ form a second bond between the carbon atoms they are attached to and R₃ is hydrogen, deuterium, or —OH. In some embodiments, R₂ and R₅ form a second bond between the carbon atoms they are attached to and R₃ is hydrogen. In some other embodiments, R₂ and R₅ form a second bond between the carbon atoms they are attached to and R₃ is deuterium. In still some other embodiments, R₂ and R₅ form a second bond between the carbon atoms they are attached to and R₃ is OH.

In compounds of any one of the aspects, R₂ and R₅ form a second bond between the carbon atoms they are attached to and R₄ can be hydrogen, deuterium, C₁-C₈alkyl, or —OH. For example, R₂ and R₅ form a second bond between the carbon atoms they are attached to and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₂ and R₅ form a second bond between the carbon atoms they are attached to and R₄ is hydrogen, deuterium, or —OH. In some embodiments, R₂ and R₅ form a second bond between the carbon atoms they are attached to and R₄ is hydrogen. In some other embodiments, R₂ and R₅ form a second bond between the carbon atoms they are attached to and R₄ is deuterium. In still some other embodiments, R₂ and R₅ form a second bond between the carbon atoms they are attached to and R₄ is OH.

In some embodiments of any one of the aspects described herein, R₅ and R₄ form a second bond between the carbon atoms they are attached to. In some compounds of the various aspects described herein, R₂ and R₅ form a second bond between the carbon atoms they are attached to.

In compounds of any one of the aspects, R₃ and R₄ form a second bond between the carbon atoms they are attached to and R₂ can be hydrogen, deuterium, C₁-C₈alkyl, or —OH. For example, R₃ and R₄ form a second bond between the carbon atoms they are attached to and R₂ can be hydrogen, deuterium, methyl, ethyl, propyl, i-propyl, n-butyl, t-butyl, n-pentyl, hexyl, heptyl, octyl, or OH. In some embodiments of any one of the aspects, R₅ and R₄ form a second bond between the carbon atoms they are attached to and R₂ is hydrogen, deuterium, or —OH. In some embodiments, R₃ and R₄ form a second bond between the carbon atoms they are attached to and R₂ is hydrogen.

In some other embodiments, R₃ and R₄ form a second bond between the carbon atoms they are attached to and R₂ is deuterium. In still some other embodiments, R₃ and R₄ form a second bond between the carbon atoms they are attached to and R₂ is OH.

In compounds of any one of the aspects, R₃ and R₄ form a second bond between the carbon atoms they are attached to and R₅ can be hydrogen, deuterium, C₁-C₈alkyl, or —OH. For example, R₃ and R₄ form a second bond between the carbon atoms they are attached to and R₅ can be hydrogen, deuterium, methyl, ethyl, propyl, i-propyl, n-butyl, t-butyl, n-pentyl, hexyl, heptyl, octyl, or OH. In some embodiments of any one of the aspects, R₃ and R₄ form a second bond between the carbon atoms they are attached to and R₅ is hydrogen, deuterium, or —OH. In some embodiments, R₃ and R₄ form a second bond between the carbon atoms they are attached to and R₅ is hydrogen. In some other embodiments, R₂ and R₄ form a second bond between the carbon atoms they are attached to and R₅ is deuterium. In still some other embodiments, R₃ and R₄ form a second bond between the carbon atoms they are attached to and R₅ is OH.

In compounds of any one of the aspects, R₃ and R₅ form a second bond between the carbon atoms they are attached to and R₂ can be hydrogen, deuterium, C₁-C₈alkyl, or —OH. For example, R₃ and R₅ form a second bond between the carbon atoms they are attached to and R₂ can be hydrogen, deuterium, methyl, ethyl, propyl, i-propyl, n-butyl, t-butyl, n-pentyl, hexyl, heptyl, octyl, or OH. In some embodiments of any one of the aspects, R₃ and R₅ form a second bond between the carbon atoms they are attached to and R₂ is hydrogen, deuterium, or —OH. In some embodiments, R₃ and R₅ form a second bond between the carbon atoms they are attached to and R₂ is hydrogen. In some other embodiments, R₃ and R₅ form a second bond between the carbon atoms they are attached to and R₂ is deuterium. In still some other embodiments, R₃ and R₅ form a second bond between the carbon atoms they are attached to and R₂ is OH.

In compounds of any one of the aspects, R₃ and R₅ form a second bond between the carbon atoms they are attached to and R₄ can be hydrogen, deuterium, C₁-C₈alkyl, or —OH. For example, R₄ and R₅ form a second bond between the carbon atoms they are attached to and R₄ can be hydrogen, deuterium, methyl, ethyl, propyl, i-propyl, n-butyl, t-butyl, n-pentyl, hexyl, heptyl, octyl, or OH. In some embodiments of any one of the aspects, R₃ and R₅ form a second bond between the carbon atoms they are attached to and R₄ is hydrogen, deuterium, or —OH. In some embodiments, R₃ and R₅ form a second bond between the carbon atoms they are attached to and R₄ is hydrogen. In some other embodiments, R₃ and R₅ form a second bond between the carbon atoms they are attached to and R₄ is deuterium. In still some other embodiments, R₃ and R₅ form a second bond between the carbon atoms they are attached to and R₄ is OH.

In some embodiments of any one of the aspects, R₆ is substituted or unsubstituted aryl. In some embodiments of any one of the aspects, R₆ is substituted or unsubstituted phenyl. In some embodiments of any one of the aspects, R₆ is unsubstituted phenyl. In some embodiments of any one of the aspects, R₆ is substituted phenyl. In some embodiments of any one of the aspects, R₆ is phenyl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments of any one of the aspects, R₆ is phenyl substituted with at least one substituent selected from alkyl, hydroxy, alkoxy, halogen, and haloalkyl. In some embodiments of any one of the aspects, R₆ is phenyl substituted with at least one halogen substituent. In some embodiments of any one of the aspects, R₆ is phenyl substituted with at least one fluoro substituent. In some embodiments of any one of the aspects, R₆ is 4-fluorophenyl.

In some embodiments of any one of the aspects, R₆ is substituted or unsubstituted heteroaryl. In some embodiments of any one of the aspects, R₆ is unsubstituted heteroaryl. In some embodiments of any one of the aspects, R₆ is substituted heteroaryl. In some embodiments of any one of the aspects, R₆ is heteroaryl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments of any one of the aspects, R₆ is heteroaryl substituted with at least one substituent selected from alkyl, hydroxy, alkoxy, halogen, and haloalkyl. In some embodiments of any one of the aspects, R₆ is heteroaryl substituted with at least one halogen substituent. In some embodiments of any one of the aspects, R₆ is a heteroaryl selected from thienyl, furyl, thiadiazolyl, benzothiadiazolyl, pyrrolyl, imidazolyl, oxazolyl, pyrazolyl, isothiazolyl, isoxazolyl, pyridyl, pyrazinyl, pyrimidinyl, pyridazinyl, pyrazolo-pyrimidinyl, triazolo-pyrimidinyl, and imidazo-pyrimidinyl.

In some embodiments of any one of the aspects described herein, R₆ is C₆-C₁₀aryl optionally substituted with 1, 2, 3, or 4 R₉ groups. In some embodiments of any one of the aspects described herein, R₆ is C₆-C₁₀aryl substituted with 1, 2, or 3 R₉ groups. In some embodiments of any one of the aspects described herein, R₆ is phenyl substituted with 1, 2, or 3 R₉ groups. In some embodiments of any one of the aspects described herein, R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆-alkyl, C₁₋₆haloalkyl, C₁₋₆alkoxy, and C₁₋₆haloalkoxy. In some embodiments of any one of the aspects described herein, R₆ is phenyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is phenyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is phenyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is phenyl substituted with 1 R₉ group and R₉ is halogen. In some embodiments of any one of the aspects described herein, R₆ is phenyl substituted with 1 R₉ group and R₉ is fluoro. In some embodiments of any one of the aspects described herein, R₆ is phenyl substituted with 1 R₉ group and R₉ is C₁₋₆alkyl optionally substituted with one, two, or three groups independently selected from halogen. In some embodiments of any one of the aspects described herein, R₆ is phenyl substituted with 1 R₉ group and R₉ is unsubstituted C₁₋₆alkyl. In some embodiments of any one of the aspects described herein, R₆ is phenyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is phenyl substituted with 1 R₉ group and R₉ is unsubstituted phenyl.

In some embodiments of any one of the aspects described herein, R₆ is C₂-C₉heteroaryl optionally substituted with 1, 2, or 3 R₉ groups. In some embodiments of any one of the aspects described herein, R₆ is phenyl substituted with 1, 2, or 3 R₉ groups. In some embodiments of any one of the aspects described herein, R₆ is C₂-C₉heteroaryl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, R₆ is C₂-C₉heteroaryl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, R₆ is C₂-C₉heteroaryl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is C₂-C₉heteroaryl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is C₂-C₉heteroaryl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is C₂-C₉heteroaryl substituted with 1 R₉ group and R₉ is halogen. In some embodiments of any one of the aspects described herein, R₆ is C₂-C₉heteroaryl substituted with 1 R₉ group and R₉ is fluoro. In some embodiments of any one of the aspects described herein, R₆ is C₂-C₉heteroaryl substituted with 1 R₉ group and R₉ is C₁₋₆alkyl optionally substituted with one, two, or three groups independently selected from halogen. In some embodiments of any one of the aspects described herein, R₆ is C₂-C₉heteroaryl substituted with 1 R₉ group and R₉ is unsubstituted C₁₋₆alkyl. In some embodiments of any one of the aspects described herein, R₆ is C₂-C₉heteroaryl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is C₂-C₉heteroaryl substituted with 1 R₉ group and R₉ is unsubstituted phenyl.

In some embodiments of any one of the aspects described herein, R₆ is unsubstituted C₂-C₉heteroaryl.

In some embodiments of any one of the aspects described herein, R₆ is pyridyl optionally substituted with 1, 2, or 3 R₉ groups. In some embodiments of any one of the aspects described herein, R₆ is phenyl substituted with 1, 2, or 3 R₉ groups. In some embodiments of any one of the aspects described herein, R₆ is pyridyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, R₆ is pyridyl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, R₆ is pyridyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is pyridyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is pyridyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is pyridyl substituted with 1 R₉ group and R₉ is halogen. In some embodiments of any one of the aspects described herein, R₆ is pyridyl substituted with 1 R₉ group and R₉ is fluoro. In some embodiments of any one of the aspects described herein, R₆ is pyridyl substituted with 1 R₉ group and R₉ is C₁₋₆alkyl optionally substituted with one, two, or three groups independently selected from halogen. In some embodiments of any one of the aspects described herein, R₆ is pyridyl substituted with 1 R₉ group and R₉ is unsubstituted C₁₋₆alkyl. In some embodiments of any one of the aspects described herein, R₆ is pyridyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, R₆ is pyridyl substituted with 1 R₉ group and R₉ is unsubstituted phenyl. In some embodiments of any one of the aspects described herein, R₆ is unsubstituted pyridyl.

In some embodiments of any one of the aspects, R₆ is a substituted or unsubstituted alkyl. For example, R₆ is a substituted or unsubstituted C₁-C₆alkyl. In some embodiments of any one of the aspects, R₆ can be methyl, ethyl, propyl, i-propyl, n-butyl, t-butyl, n-pentyl or hexyl. For example, R₆ can be n-butyl or t-butyl. In some embodiments of any one of the aspects, R₆ is n-butyl. In some other embodiments of any one of the aspects, R₆ is t-butyl.

In compounds of the any one of the aspects described herein, R₇ can be H, substituted or unsubstituted alkyl, or —C(O)NR₁₀R₁₁. For example, R₇ can be hydrogen or substituted or unsubstituted C₁-C₈alkyl. In some embodiments of any one of the aspects, R₇ is hydrogen. In some embodiments of any one of the aspects, R₇ is substituted or unsubstituted C₁-C₈alkyl. In some embodiments of any one of the aspects, R₇ is unsubstituted C₁-C₈alkyl. In some embodiments of any one of the aspects, R₇ is —CH₃. In some embodiments of any one of the aspects, R₇ is —C(O)NR₁₀R₁₁. In some embodiments of any one of the aspects, R₇ is substituted or unsubstituted C₁-C₈alkyl. In some embodiments of any one of the aspects, R₇ is —C(O)NR₁₀R₁₁, and R₁₀ and R₁₁ are independently substituted or unsubstituted C₁-C₈alkyl. In some embodiments of any one of the aspects, R₇ is —C(O)NR₁₀R₁₁, and R₁₀ and R₁₁ are each —CH₃. In some embodiments of any one of the aspects, R₇ is —C(O)NR₁₀R₁₁. R₁₀ is hydrogen, and R₁₁ is substituted or unsubstituted C₁-C₈alkyl. In some embodiments of any one of the aspects, R₇ is —C(O)NR₁₀R₁₁, R₁₀ is hydrogen, and R₁₁ is —CH₃. In some embodiments of any one of the aspects, R₇ is —C(O)NR₁₀R₁₁, R₁₀ is substituted or unsubstituted aryl, and R₁₁ is substituted or unsubstituted C₁-C₈alkyl. In some embodiments of any one of the aspects, R₇ is —C(O)NR₁₀R₁₁, R₁₀ is substituted or unsubstituted aryl, and R₁₁ is hydrogen.

In the compounds described herein, R₈ can be H or OH. For example, in some embodiments of any one of the aspects, R₈ is H. In some other embodiments of any one of the aspects, R₈ is OH.

In some embodiments of any one of the aspects described herein, the compound of Formula (I) is of Formula (Ia):

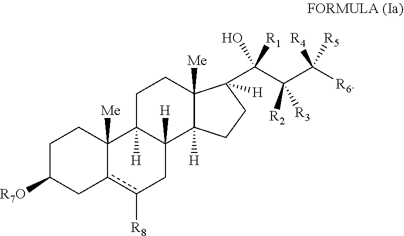

In some embodiments of any one of the aspects described herein, the compound of Formula (I) is of Formula (Ib):

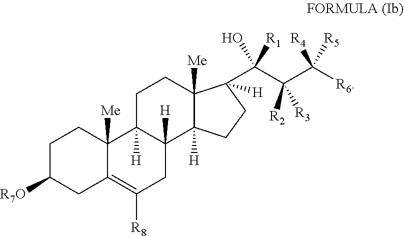

In some embodiments of any one of the aspects described herein, the compound of Formula (I) is of Formula (Ic):

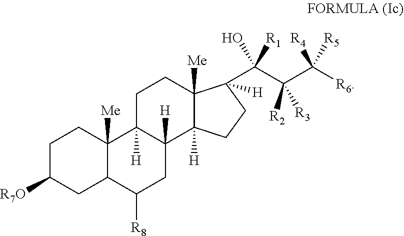

In some embodiments of any one of the aspects described herein, the compound of Formula (I) is of Formula (II):

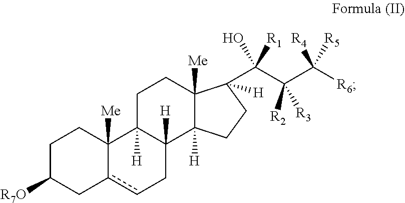

wherein:
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
R₆ is substituted or unsubstituted aryl or substituted or unsubstituted heteroaryl;
[symbol] is a single or double bond;
R₂, R₃, R₄, and R₅ are independently hydrogen, deuterium, or —OH;
R₇ is hydrogen, substituted or unsubstituted C₁-C₈alkyl, or —C(O)NR₁₀R₁₁; and
R₁₀ and R₁₁ are independently hydrogen, substituted or unsubstituted C₁-C₈alkyl, or substituted or unsubstituted aryl.

In some embodiments is a compound of Formula (II) wherein [symbol] is a single bond. In some embodiments is a compound of Formula (II) wherein [symbol] is a double bond.

In some embodiments is a compound of Formula (II), wherein R₆ is substituted or unsubstituted aryl. In some embodiments is a compound of Formula (II), wherein R₆ is substituted or unsubstituted phenyl in some embodiments is a compound of Formula (II), wherein R₆ is unsubstituted phenyl. In some embodiments is a compound of Formula (II), wherein R₆ is substituted phenyl. In some embodiments is a compound of Formula (II), wherein R₆ is phenyl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments is a compound of Formula (II), wherein R₆ is phenyl substituted with at least one substituent selected from alkyl, hydroxy, alkoxy, halogen, and haloalkyl. In some embodiments is a compound of Formula (II), wherein R₆ is phenyl substituted with at least one halogen substituent. In some embodiments is a compound of Formula (II), wherein R₆ is phenyl substituted with at least one fluoro substituent. In some embodiments is a compound of Formula (II), wherein R₆ is 4-fluorophenyl.

In some embodiments is a compound of Formula (II), wherein R₆ is substituted or unsubstituted heteroaryl. In some embodiments is a compound of Formula (II), wherein R₆ is unsubstituted heteroaryl. In some embodiments is a compound of Formula (II), wherein R₆ is substituted heteroaryl. In some embodiments is a compound of Formula (II), wherein R₆ is heteroaryl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments is a compound of Formula (II), wherein R₆ is heteroaryl substituted with at least one substituent selected from alkyl, hydroxy, alkoxy, halogen, and haloalkyl. In some embodiments is a compound of Formula (II), wherein R₆ is heteroaryl substituted with at least one halogen substituent. In some embodiments is a compound of Formula (II), wherein R₆ is a heteroaryl selected from thienyl, furyl, thiadiazolyl, benzothiadiazolyl, pyrrolyl, imidazolyl, oxazolyl, pyrazolyl, isothiazolyl, isoxazolyl, pyridyl, pyrazinyl, pyrimidinyl, pyridazinyl, pyrazolo-pyrimidinyl, triazolo-pyrimidinyl, and imidazo-pyrimidinyl.

In some embodiments is a compound of Formula (II), wherein R₂, R₃, R₄, and R₅ are independently hydrogen or deuterium. In some embodiments is a compound of Formula (II), wherein R₂, R₃, R₄, and R₅ are each deuterium. In some embodiments is a compound of Formula (II), wherein R₂, R₃, R₄, and R₅ are each hydrogen. In some embodiments is a compound of Formula (II), wherein R₂ is —OH, and R₃, R₄, and R₅ are each hydrogen. In some embodiments is a compound of Formula (I), wherein R₃ is —OH, and R₂, R₄, and R₅ are each hydrogen. In some embodiments is a compound of Formula (II), wherein R₂ and R₄ are each —OH, and R₃ and R₅ are each hydrogen. In some embodiments is a compound of Formula (II), wherein R₂ and R₅ are each —OH, and R₃ and R₄ are each hydrogen. In some embodiments is a compound of Formula (II), wherein R₃ and R₄ are each —OH, and R₂ and R₅ are each hydrogen. In some embodiments is a compound of Formula (II), wherein R₂ and R₅ are each —OH, and R₂ and R₄ are each hydrogen.

In some embodiments is a compound of Formula (II), wherein R₁ is substituted or unsubstituted C₁-C₈alkyl. In some embodiments is a compound of Formula (I), wherein R₁ is substituted C₁-C₈alkyl. In some embodiments is a compound of Formula (II), wherein R₁ is —CF₃. In some embodiments is a compound of Formula (II), wherein R₁ is unsubstituted C₁-C₈alkyl. In some embodiments is a compound of Formula (I), wherein R₁ is unsubstituted C₁-C₄alkyl. In some embodiments is a compound of Formula (II), wherein R₁ is —CH₃. In some embodiments is a compound of Formula (II), wherein R₁ is —CH₂CH₃. In some embodiments is a compound of Formula (II), wherein R₁ is substituted or unsubstituted aryl. In some embodiments is a compound of Formula (II), wherein R₁ is unsubstituted phenyl. In some embodiments is a compound of Formula (II), wherein R₁ is substituted or unsubstituted C₁-C₈alkyl or substituted or unsubstituted phenyl.

In some embodiments is a compound of Formula (II), wherein R₇ is hydrogen or substituted or unsubstituted C₁-C₈alkyl. In some embodiments is a compound of Formula (II), wherein R₇ is hydrogen. In some embodiments is a compound of Formula (II), wherein R₇ is substituted or unsubstituted C₁-C₈alkyl. In some embodiments is a compound of Formula (II), wherein R₇ is unsubstituted C₁-C₈alkyl. In some embodiments is a compound of Formula (II), wherein R₇ is —CH₃. In some embodiments is a compound of Formula (II), wherein R₇ is —C(O)NR₁₀R₁₁. In some embodiments is a compound of Formula (II), wherein R₇ is substituted or unsubstituted C₁-C₈alkyl. In some embodiments is a compound of Formula (II), wherein R₇ is —C(O)NR₁₀R₁₁, and R₁₀ and R₁₁ are independently substituted or unsubstituted C₁-C₈alkyl in some embodiments is a compound of Formula (II), wherein R₇ is —C(O)NR₁₀R₁₁, and R₁₀ and R₁₁ are each —CH₃. In some embodiments is a compound of Formula (II), wherein R₇ is —C(O)NR₁₀R₁₁, R₁₀ is hydrogen, and R₁₁ is substituted or unsubstituted C₁-C₈alkyl. In some embodiments is a compound of Formula (II), wherein R₇ is —C(O)NR₁₀R₁₁, R₁₀ is hydrogen, and R₁₁ is —CH₃. In some embodiments is a compound of Formula (II), wherein R₇ is —C(O)NR₁₀R₁₁, R₁₀ is substituted or unsubstituted aryl, and R₁₁ is substituted or unsubstituted C₁-C₈alkyl. In some embodiments is a compound of Formula (II), wherein R₇ is —C(O)NR₁₀R₁₁, R₁₀ is substituted or unsubstituted aryl, and R₁₁ is hydrogen.

In some embodiments of any one of the aspects described herein, the compound of Formula (I) is of Formula (IIa):

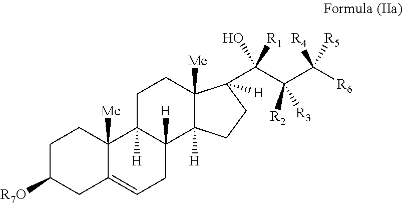

wherein:
[symbol] is a single or double bond;
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
R₆ is substituted or unsubstituted aryl or substituted or unsubstituted heteroaryl;
R₂, R₃, R₄, and R₅ are independently hydrogen, deuterium, or —OH;
R₇ is hydrogen, substituted or unsubstituted C₁-C₈alkyl, or —C(O)NR₁₀R₁₁; and
R₁₀ and R₁₁ are independently hydrogen, substituted or unsubstituted C₁-C₈alkyl, or substituted or unsubstituted aryl.

In some embodiments is a compound of Formula (IIa), wherein R₆ is substituted or unsubstituted aryl. In some embodiments is a compound of Formula (IIa), wherein R₆ is substituted or unsubstituted phenyl. In some embodiments is a compound of Formula (IIa), wherein R₆ is unsubstituted phenyl. In some embodiments is a compound of Formula (IIa), wherein R₆ is substituted phenyl. In some embodiments is a compound of Formula (IIa), wherein R₆ is phenyl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments is a compound of Formula (IIa), wherein R₆ is phenyl substituted with at least one substituent selected from alkyl, hydroxy, alkoxy, halogen, and haloalkyl. In some embodiments is a compound of Formula (IIa), wherein R₆ is phenyl substituted with at least one halogen substituent. In some embodiments is a compound of Formula (IIa), wherein R₆ is phenyl substituted with at least one fluoro substituent.

In some embodiments is a compound of Formula (IIa), wherein R₆ is substituted or unsubstituted heteroaryl. In some embodiments is a compound of Formula (IIa), wherein R₆ is unsubstituted heteroaryl. In some embodiments is a compound of Formula (IIa), wherein R₆ is substituted heteroaryl. In some embodiments is a compound of Formula (IIa), wherein R₆ is heteroaryl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments is a compound of Formula (IIa), wherein R₆ is heteroaryl substituted with at least one substituent selected from alkyl, hydroxy, alkoxy, halogen, and haloalkyl. In some embodiments is a compound of Formula (IIa), wherein R₆ is heteroaryl substituted with at least one halogen substituent. In some embodiments is a compound of Formula (IIa), wherein R₆ is a heteroaryl selected from thienyl, furyl, thiadiazolyl, benzothiadiazolyl, pyrrolyl, imidazolyl, oxazolyl, pyrazolyl, isothiazolyl, isoxazolyl, pyridyl, pyrazinyl, pyrimidinyl, pyridazinyl, pyrazolo-pyrimidinyl, triazolo-pyrimidinyl, and imidazo-pyrimidinyl.

In some embodiments is a compound of Formula (IIa), wherein R₂, R₃, R₄, and R₃ are each deuterium. In some embodiments is a compound of Formula (IIa), wherein R₂, R₃, R₄, and R₅ are each hydrogen. In some embodiments is a compound of Formula (IIa), wherein R₂ is —OH, and R₃, R₄, and R₅ are each hydrogen. In some embodiments is a compound of Formula (IIa), wherein R₃ is —OH, and R₂, R₄, and R₅ are each hydrogen. In some embodiments is a compound of Formula (IIa), wherein R₂ and R₄ are each —OH, and R₃ and R₅ are each hydrogen. In some embodiments is a compound of Formula (IIa), wherein R₂ and R₅ are each —OH, and R₃ and R₄ are each hydrogen. In some embodiments is a compound of Formula (IIa), wherein R₂ and R₄ are each —OH, and R₂ and R₅ are each hydrogen. In some embodiments is a compound of Formula (IIa), wherein R₃ and R₅ are each —OH, and R₂ and R₄ are each hydrogen.

In some embodiments is a compound of Formula (IIa), wherein R₁ is substituted or unsubstituted C₁-C₈alkyl. In some embodiments is a compound of Formula (IIa), wherein R₁ is substituted C₁-C₈alkyl. In some embodiments is a compound of Formula (IIa), wherein R₁ is —CF₃. In some embodiments is a compound of Formula (IIa), wherein R₁ is unsubstituted C₁-C₈alkyl. In some embodiments is a compound of Formula (IIa), wherein R₁ is —CH₃. In some embodiments is a compound of Formula (IIa), wherein R₁ is —CH₂CH₃. In some embodiments is a compound of Formula (IIa), wherein R₁ is substituted or unsubstituted aryl. In some embodiments is a compound of Formula (IIa), wherein R₁ is unsubstituted phenyl.

In some embodiments is a compound of Formula (IIa), wherein R₇ is hydrogen. In some embodiments is a compound of Formula (IIa), wherein R₇ is substituted or unsubstituted C₁-C₈alkyl. In some embodiments is a compound of Formula (IIa), wherein R₇ is —CH₃. In some embodiments is a compound of Formula (IIa), wherein R₇ is —C(O)NR₁₀R₁₁. In some embodiments is a compound of Formula (IIa), wherein R₇ is substituted or unsubstituted C₁-C₈alkyl. In some embodiments is a compound of Formula (IIa), wherein R₇ is —C(O)NR₁₀R₁₁, and R₁₀ and R₁₁ are independently substituted or unsubstituted C₁-C₈alkyl. In some embodiments is a compound of Formula (IIa), wherein R₇ is —C(O)NR₁₀R₁₁, and R₁₀ and R₁₁ are each —CH₃. In some embodiments is a compound of Formula (IIa), wherein R₇ is —C(O)NR₁₀R₁₁, R₁₀ is hydrogen, and R₁₁ is substituted or unsubstituted C₁-C₈alkyl. In some embodiments is a compound of Formula (IIa), wherein R₇ is —C(O)NR₁₀R₁₁, R₁₀ is hydrogen, and R₁₁ is —CH₃. In some embodiments is a compound of Formula (IIa), wherein R₇ is —C(O)NR₁₀R₁₁, R₁₀ is substituted or unsubstituted aryl, and R₁₁ is substituted or unsubstituted C₁-C₈alkyl. In some embodiments is a compound of Formula (IIa), wherein R₇ is —C(O)NR₁₀R₁₁, R₁₀ is substituted or unsubstituted aryl, and R₁₁ is hydrogen.

In some embodiments of any one of the aspects described herein, the compound of Formula (I) is a compound of Formula (IIb):

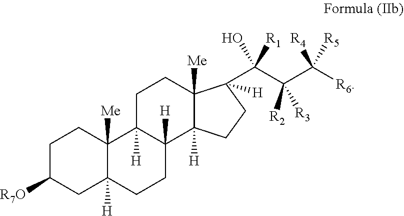

wherein:
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
R₆ is substituted or unsubstituted aryl or substituted or unsubstituted heteroaryl;
R₂, R₃, R₄, and R₅ are independently hydrogen, deuterium, or —OH;
R₇ is hydrogen, substituted or unsubstituted C₁-C₈alkyl, or —C(O)NR₁₀R₁₁; and
R₁₀ and R₁₁ are independently hydrogen, substituted or unsubstituted C₁-C₈alkyl, or substituted or unsubstituted aryl.

In some embodiments is a compound of Formula (IIb), wherein R₆ is substituted or unsubstituted aryl. In some embodiments is a compound of Formula (IIb), wherein R₆ is substituted or unsubstituted phenyl. In some embodiments is a compound of Formula (IIb), wherein R₆ is unsubstituted phenyl. In some embodiments is a compound of Formula (IIb), wherein R₆ is substituted phenyl. In some embodiments is a compound of Formula (IIb), wherein R₆ is phenyl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments is a compound of Formula (IIb), wherein R₆ is phenyl substituted with at least one substituent selected from alkyl, hydroxy, alkoxy, halogen, and haloalkyl. In some embodiments is a compound of Formula (IIb), wherein R₆ is phenyl substituted with at least one halogen substituent. In some embodiments is a compound of Formula (IIb), wherein R₆ is phenyl substituted with at least one fluoro substituent.

In some embodiments is a compound of Formula (IIb), wherein R₆ is substituted or unsubstituted heteroaryl. In some embodiments is a compound of Formula (IIb), wherein R₆ is unsubstituted heteroaryl. In some embodiments is a compound of Formula (IIb), wherein R₆ is substituted heteroaryl. In some embodiments is a compound of Formula (IIb), wherein R₆ is heteroaryl substituted with at least one substituent selected from amide, ester, alkyl, cycloalkyl, heteroalkyl, aryl, heteroaryl, heterocycloalkyl, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, and amido. In some embodiments is a compound of Formula (IIb), wherein R₆ is heteroaryl substituted with at least one substituent selected from alkyl, hydroxy, alkoxy, halogen, and haloalkyl. In some embodiments is a compound of Formula (IIb), wherein R₆ is heteroaryl substituted with at least one halogen substituent. In some embodiments is a compound of Formula (IIb), wherein R₆ is a heteroaryl selected from thienyl, furyl, thiadiazolyl, benzothiadiazolyl, pyrrolyl, imidazolyl, oxazolyl, pyrazolyl, isothiazolyl, isoxazolyl, pyridyl, pyrazinyl, pyrimidinyl, pyridazinyl, pyrazolo-pyrimidinyl, triazolo-pyrimidinyl, and imidazo-pyrimidinyl.

In some embodiments is a compound of Formula (IIb), wherein R₂, R₃, R₄, and R₅ are each deuterium. In some embodiments is a compound of Formula (IIb), wherein R₂, R₃, R₄, and R₅ are each hydrogen. In some embodiments is a compound of Formula (IIb), wherein R₂ is —OH, and R₃, R₄, and R₅ are each hydrogen. In some embodiments is a compound of Formula (IIb), wherein R₃ is —OH, and R₂, R₄, and R₅ are each hydrogen. In some embodiments is a compound of Formula (IIb), wherein R₂ and R₄ are each —OH, and R₃ and R₅ are each hydrogen. In some embodiments is a compound of Formula (IIb), wherein R₂ and R₅ are each —OH, and R₃ and R₄ are each hydrogen. In some embodiments is a compound of Formula (IIb), wherein R₃ and R₅ are each —OH, and R₂ and R₅ are each hydrogen. In some embodiments is a compound of Formula (IIb), wherein R₃ and R₅ are each —OH, and R₂ and R₄ are each hydrogen.

In some embodiments is a compound of Formula (IIb), wherein R₁ is substituted or unsubstituted C₁-C₈alkyl. In some embodiments is a compound of Formula (IIb), wherein R₁ is substituted C₁-C₈alkyl. In some embodiments is a compound of Formula (IIb), wherein R₁ is CF₃. In some embodiments is a compound of Formula (IIb), wherein R₁ is unsubstituted C₁-C₈alkyl. In some embodiments is a compound of Formula (IIb), wherein R₁ is —CH₃. In some embodiments is a compound of Formula (IIb), wherein R₁ is —CH₂CH₃. In some embodiments is a compound of Formula (IIb), wherein R₁ is substituted or unsubstituted aryl. In some embodiments is a compound of Formula (IIb), wherein R₁ is unsubstituted phenyl.

In some embodiments is a compound of Formula (IIb), wherein R₇ is hydrogen. In some embodiments is a compound of Formula (IIb), wherein R₇ is substituted or unsubstituted C₁-C₈alkyl. In some embodiments is a compound of Formula (IIb), wherein R₇ is —CH₃. In some embodiments is a compound of Formula (IIb), wherein R₇ is —C(O)NR₁₀R₁₁. In some embodiments is a compound of Formula (IIb), wherein R₇ is substituted or unsubstituted C₁-C₈alkyl. In some embodiments is a compound of Formula (IIb), wherein R₇ is —C(O)NR₁₀R₁₁, and R₁₀ and R₁₁ are independently substituted or unsubstituted C₁-C₈alkyl. In some embodiments is a compound of Formula (IIb), wherein R₇ is —C(O)NR₁₀R₁₁, and R₁₀ and R₁₁ are each —CH₃. In some embodiments is a compound of Formula (IIb), wherein R₇ is —C(O)NR₁₀R₁₁, R₁₀ is hydrogen, and R₁₁ is substituted or unsubstituted C₁-C₈alkyl. In some embodiments is a compound of Formula (IIb), wherein R₇ is —C(O)NR₁₀R₁₁, R₁₀ is hydrogen, and R₁₁ is —CH₃. In some embodiments is a compound of Formula (IIb), wherein R₇ is —C(O)NR₁₀R₁₁, R₁₀ is substituted or unsubstituted aryl, and R₁₁ is substituted or unsubstituted C₁-C₈alkyl. In some embodiments is a compound of Formula (IIb), wherein R₇ is —C(O)NR₁₀R₁₁, R₁₀ is substituted or unsubstituted aryl, and R₁₁ is hydrogen.

In some embodiments of any one of the aspects described herein, the compound of Formula (I) is of Formula (III).

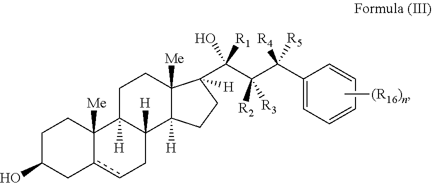

wherein:
[symbol] is a single or double bond;
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl;
R₂, R₃, R₄, and R₅ are independently hydrogen, deuterium, or —OH; and n is 0, 1, 2 or 3.

In some embodiments is a compound of Formula (III) wherein [symbol] is a single bond. In some embodiments is a compound of Formula (III) wherein [symbol] is a double bond.

In some embodiments is a compound of Formula (III), wherein n is 0. In some embodiments is a compound of Formula (III), wherein n is 1 and R₁₆ is halogen. In some embodiments is a compound of Formula (III), wherein n is 1 and R₁₆ is F. In some embodiments is a compound of Formula (II), wherein n is 1 and R₁₆ is Cl in some embodiments is a compound of Formula (III), wherein n is 1 and R₁₆ is Br. In some embodiments is a compound of Formula (III), wherein n is 1 and R₁₆ is hydroxy. In some embodiments is a compound of Formula (III), wherein n is 1 and R₁₆ is substituted or unsubstituted C₁-C₈alkyl. In some embodiments is a compound of Formula (III), wherein n is 1 and R₁₆ is unsubstituted C₁-C₈alkyl. In some embodiments is a compound of Formula (III), wherein n is 1 and R₁₆ is substituted or unsubstituted C₁-C₈alkoxy. In some embodiments is a compound of Formula (III), wherein n is 2 and each R₁₆ is halogen. In some embodiments is a compound of Formula (III), wherein n is 2 and each R₁₆ is F. In some embodiments is a compound of Formula (III), wherein n is 2 and each R₁₆ is Cl. In some embodiments is a compound of Formula (III), wherein n is 2 and each R₁₆ is Br. In some embodiments is a compound of Formula (III), wherein n is 2 and one R₁₆ is halogen and one R₁₆ is hydroxy. In some embodiments is a compound of Formula (III), wherein n is 2 and each R₁₆ is hydroxy. In some embodiments is a compound of Formula (III), wherein n is 2 and one R₁₆ is halogen and one R₁₆ is substituted or unsubstituted C₁-C₈alkyl. In some embodiments is a compound of Formula (III), wherein n is 2 and each R₁₆ is substituted or unsubstituted C₁-C₈alkyl. In some embodiments is a compound of Formula (III), wherein n is 2 and each R₁₆ is unsubstituted C₁-C₈alkyl. In some embodiments is a compound of Formula (III), wherein n is 2 and one R₁₆ is halogen and one R₁₆ is substituted or unsubstituted C₁-C₈alkoxy. In some embodiments is a compound of Formula (III), wherein n is 2 and each R₁₆ is substituted or unsubstituted C₁-C₈alkoxy.

In some embodiments is a compound of Formula (III), wherein R₂, R₃, R₄, and R₅ are each deuterium. In some embodiments is a compound of Formula (III), wherein R₂, R₃, R₄, and R₅ are each hydrogen. In some embodiments is a compound of Formula (III), wherein R₂ is —OH, and R₃, R₄, and R₅ are each hydrogen. In some embodiments is a compound of Formula (III), wherein R₃ is —OH, and Ra, R₄, and R₅ are each hydrogen. In some embodiments is a compound of Formula (III), wherein R₂ and R₄ are each —OH, and R₃ and R₅ are each hydrogen. In some embodiments is a compound of Formula (III), wherein R₂ and R₅ are each —OH, and R₃ and R₄ are each hydrogen. In some embodiments is a compound of Formula (III), wherein R₃ and R₄ are each —OH, and R₂ and R₅ are each hydrogen in some embodiments is a compound of Formula (III), wherein R₃ and R₅ are each OH, and R₂ and R₄ are each hydrogen.

In some embodiments is a compound of Formula (III), wherein R₁ is substituted or unsubstituted C₁-C₈alkyl. In some embodiments is a compound of Formula (III), wherein R₁ is substituted C₁-C₈alkyl. In some embodiments is a compound of Formula (III), wherein R₁ is —CF₃. In some embodiments is a compound of Formula (III), wherein R₁ is unsubstituted C₁-C₈alkyl. In some embodiments is a compound of Formula (III), wherein R₁ is —CH₃. In some embodiments is a compound of Formula (III), wherein R₁ is —CH₂CH₃. In some embodiments is a compound of Formula (III), wherein R₁ is substituted or unsubstituted aryl. In some embodiments is a compound of Formula (III), wherein R₁ is unsubstituted phenyl.

In some embodiments of any one of the aspects described herein, the compound of Formula (I) is of Formula (IIIa):

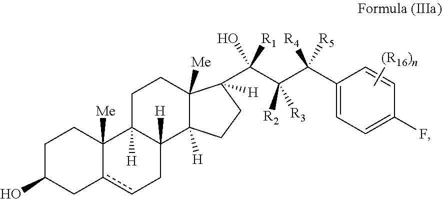

wherein:
[symbol] is a single or double bond;
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl, each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl;
R₂, R₃, R₄, and R₅ are independently hydrogen, deuterium, or —OH; and
n is 0, 1, or 2.

In some embodiments is a compound of Formula (IIIa) wherein [symbol] is a single bond. In some embodiments is a compound of Formula (IIIa) wherein [symbol] is a double bond.

In some embodiments is a compound of Formula (IIIa), wherein n is 0. In some embodiments is a compound of Formula (IIIa), wherein n is 1 and R₁₆ is halogen. In some embodiments is a compound of Formula (IIIa), wherein n is 1 and R₁₆ is F. In some embodiments is a compound of Formula (IIIa), wherein n is 1 and R₁₆ is Cl. In some embodiments is a compound of Formula (IIIa), wherein n is 1 and R₁₆ is Br. In some embodiments is a compound of Formula (IIIa), wherein n is 1 and R₁₆ is hydroxy. In some embodiments is a compound of Formula (IIIa), wherein n is 1 and R₁₆ is substituted or unsubstituted C₁-C₈alkyl. In some embodiments is a compound of Formula (IIIa), wherein n is 1 and R₁₆ is unsubstituted C₁-C₈alkyl. In some embodiments is a compound of Formula (IIIa), wherein n is 1 and R₁₆ is substituted or unsubstituted C₁-C₈alkoxy. In some embodiments is a compound of Formula (IIIa), wherein n is 2 and each R₁₆ is halogen. In some embodiments is a compound of Formula (IIIa), wherein n is 2 and each R₁₆ is F. In some embodiments is a compound of Formula (IIIa), wherein n is 2 and each R₁₆ is Cl. In some embodiments is a compound of Formula (IIIa), wherein n is 2 and each R₁₆ is Br. In some embodiments is a compound of Formula (IIIa), wherein n is 2 and one R₁₆ is halogen and one R₁₆ is hydroxy. In some embodiments is a compound of Formula (IIIa), wherein n is 2 and each R₁₆ is hydroxy. In some embodiments is a compound of Formula (IIIa), wherein n is 2 and one R₁₆ is halogen and one R₁ is substituted or unsubstituted C₁-C₈alkyl. In some embodiments is a compound of Formula (IIIa), wherein n is 2 and each R₁₆ is substituted or unsubstituted C₁-C₈alkyl. In some embodiments is a compound of Formula (IIIa), wherein n is 2 and each R₁₆ is unsubstituted C₁-C₈alkyl. In some embodiments is a compound of Formula (IIIa), wherein n is 2 and one R₁₆ is halogen and one R₁₆ is substituted or unsubstituted C₁-C₈alkoxy. In some embodiments is a compound of Formula (IIIa), wherein n is 2 and each R₁₆ is substituted or unsubstituted C₁-C₈alkoxy.

In some embodiments is a compound of Formula (IIIa), wherein R₂, R₃, R₄, and R₅ are each deuterium. In some embodiments is a compound of Formula (IIIa), wherein R₂, R₃, R₄, and R₅ are each hydrogen. In some embodiments is a compound of Formula (IIIa), wherein R₂ is —OH, and R₃, R₄, and R₅ are each hydrogen. In some embodiments is a compound of Formula (IIIa), wherein R₃ is —OH, and R₂, R₄, and R₅ are each hydrogen. In some embodiments is a compound of Formula (IIIa), wherein R₂ and R₄ are each —OH, and R₃ and R₅ are each hydrogen. In some embodiments is a compound of Formula (IIIa), wherein R₂ and R₅ are each —OH, and R₃ and R₄ are each hydrogen. In some embodiments is a compound of Formula (IIa), wherein R₃ and R₄ are each —OH, and R₂ and R₅ are each hydrogen. In some embodiments is a compound of Formula (IIa), wherein R₂ and R₅ are each —OH, and R₂ and R₄ are each hydrogen.

In some embodiments, the compound is a compound of Formula (IIIa), wherein R₁ is substituted or unsubstituted C₁-C₈alkyl. In some embodiments is a compound of Formula (IIIa), wherein R₁ is substituted C₁-C₈alkyl. In some embodiments is a compound of Formula (IIa), wherein R₁ is —CF₃. In some embodiments is a compound of Formula (IIIa), wherein R₁ is unsubstituted C₁-C₈alkyl. In some embodiments is a compound of Formula (IIIa), wherein R₁ is —CH₃. In some embodiments is a compound of Formula (IIIa), wherein R₁ is —CH₂CH₃. In some embodiments is a compound of Formula (IIIa), wherein R₁ is substituted or unsubstituted aryl. In some embodiments is a compound of Formula (IIIa), wherein R₁ is unsubstituted phenyl.

In some embodiments of any one of the aspects described herein, the compound of Formula (I) is of Formula (IV):

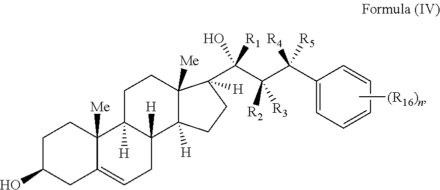

wherein:
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl;
R₂, R₃, R₄, and R₅ are independently hydrogen, deuterium, or —OH; and
n is 0, 1, 2, or 3.

In some embodiments is a compound of Formula (IV), wherein n is 0. In some embodiments is a compound of Formula (IV), wherein n is 1 and R₁₆ is halogen. In some embodiments is a compound of Formula (IV), wherein n is 1 and R₁₆ is F. In some embodiments is a compound of Formula (IV), wherein n is 1 and R₁₆ is Cl. In some embodiments is a compound of Formula (IV), wherein n is 1 and R₁₆ is Br. In some embodiments is a compound of Formula (IV), wherein n is 1 and R₁₆ is hydroxy. In some embodiments is a compound of Formula (IV), wherein n is 1 and R₁₆ is substituted or unsubstituted C₁-C₈alkyl. In some embodiments is a compound of Formula (IV), wherein n is 1 and R₁₆ is unsubstituted C₁-C₈alkyl. In some embodiments is a compound of Formula (IV), wherein n is 1 and R₁₆ is substituted or unsubstituted C₁-C₈alkoxy. In some embodiments is a compound of Formula (IV), wherein n is 2 and each R₁₆ is halogen. In some embodiments is a compound of Formula (IV), wherein n is 2 and each R₁₆ is F. In some embodiments is a compound of Formula (IV), wherein n is 2 and each R₁₆ is Cl. In some embodiments is a compound of Formula (IV), wherein n is 2 and each R₁₆ is Br. In some embodiments is a compound of Formula (IV), wherein n is 2 and one R₁₆ is halogen and one R₁₆ is hydroxy. In some embodiments is a compound of Formula (IV), wherein n is 2 and each R₁₆ is hydroxy. In some embodiments is a compound of Formula (IV), wherein n is 2 and one R₁₆ is halogen and one R₁₆ is substituted or unsubstituted C₁-C₈alkyl. In some embodiments is a compound of Formula (IV), wherein n is 2 and each R₁₆ is substituted or unsubstituted C₁-C₈alkyl. In some embodiments is a compound of Formula (IV), wherein n is 2 and each R₁₆ is unsubstituted C₁-C₈alkyl. In some embodiments is a compound of Formula (IV), wherein n is 2 and one R₁₆ is halogen and one R₁₆ is substituted or unsubstituted C₁-C₈alkoxy. In some embodiments is a compound of Formula (IV), wherein n is 2 and each R₁₆ is substituted or unsubstituted C₁-C₈alkoxy.

In some embodiments is a compound of Formula (IV), wherein R₂, R₃, R₄, and R₅ are each deuterium. In some embodiments is a compound of Formula (IV), wherein R₂, R₃, R₄, and R₅ are each hydrogen. In some embodiments is a compound of Formula (IV), wherein R₂ is —OH, and R₃, R₄, and R₅ are each hydrogen. In some embodiments is a compound of Formula (IV), wherein R₃ is —OH, and R₂, R₄, and R₅ are each hydrogen in some embodiments is a compound of Formula (IV), wherein R₂ and R₄ are each —OH, and R₃ and R₅ are each hydrogen. In some embodiments is a compound of Formula (IV), wherein R₂ and R₅ are each —OH, and R₃ and R₄ are each hydrogen. In some embodiments is a compound of Formula (IV), wherein R₃ and R₄ are each —OH, and R₂ and R₅ are each hydrogen. In some embodiments is a compound of Formula (IV), wherein R₃ and R₅ are each —OH, and R₂ and R₄ are each hydrogen.

In some embodiments is a compound of Formula (IV), wherein R₁ is substituted or unsubstituted C₁-C₈alkyl. In some embodiments is a compound of Formula (IV), wherein R₁ is substituted C₁-C₈alkyl. In some embodiments is a compound of Formula (IV), wherein R₁ is-CF₃. In some embodiments is a compound of Formula (IV), wherein R₁ is unsubstituted C₁-C₈alkyl. In some embodiments is a compound of Formula (IV), wherein R₁ is —CH₃. In some embodiments is a compound of Formula (IV), wherein R₁ is —CH₂CH₃. In some embodiments is a compound of Formula (IV), wherein R₁ is substituted or unsubstituted aryl. In some embodiments is a compound of Formula (IV), wherein R₁ is unsubstituted phenyl.

In some embodiments of any one of the aspects described herein, the compound of Formula (I) is of Formula (IVa):

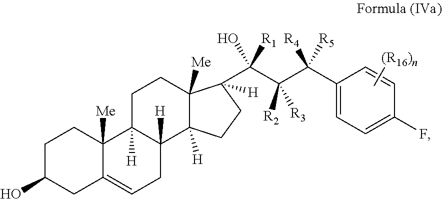

wherein:
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl;
R₂, R₃, R₄, and R₅ are independently hydrogen, deuterium, or —OH; and n is 0, 1, or 2.

In some embodiments is a compound of Formula (IVa), wherein n is 0. In some embodiments is a compound of Formula (IVa), wherein n is 1 and R₁₆ is halogen. In some embodiments is a compound of Formula (IVa), wherein n is 1 and R₁₆ is F. In some embodiments is a compound of Formula (IVa), wherein n is 1 and R₁₆ is Cl. In some embodiments is a compound of Formula (IVa), wherein n is 1 and R₁₆ is Br. In some embodiments is a compound of Formula (IVa), wherein n is 1 and R₁₆ is hydroxy. In some embodiments is a compound of Formula (IVa), wherein n is 1 and R₁₆ is substituted or unsubstituted C₁-C₈alkyl. In some embodiments is a compound of Formula (IVa), wherein n is 1 and R₁₆ is unsubstituted C₁-C₈alkyl. In some embodiments is a compound of Formula (IVa), wherein n is 1 and R₁₆ is substituted or unsubstituted C₁-C₈alkoxy. In some embodiments is a compound of Formula (IVa), wherein n is 2 and each R₁₆ is halogen. In some embodiments is a compound of Formula (IVa), wherein n is 2 and each R₁₆ is F. In some embodiments is a compound of Formula (IVa), wherein n is 2 and each R₁₆ is Cl. In some embodiments is a compound of Formula (IVa), wherein n is 2 and each R₁₆ is Br. In some embodiments is a compound of Formula (IVa), wherein n is 2 and one R₁₆ is halogen and one R₁₆ is hydroxy. In some embodiments is a compound of Formula (IVa), wherein n is 2 and each R₁₆ is hydroxy. In some embodiments is a compound of Formula (IVa), wherein n is 2 and one R₁₆ is halogen and one R₁₀ is substituted or unsubstituted C₁-C₈alkyl. In some embodiments is a compound of Formula (IVa), wherein n is 2 and each R₁₆ is substituted or unsubstituted C₁-C₈alkyl. In some embodiments is a compound of Formula (IVa), wherein n is 2 and each R₁₆ is unsubstituted C₁-C₈alkyl. In some embodiments is a compound of Formula (IVa), wherein n is 2 and one R₁₀ is halogen and one R₁ is substituted or unsubstituted C₁-C₈alkoxy. In some embodiments is a compound of Formula (IVa), wherein n is 2 and each R₁₆ is substituted or unsubstituted C₁-C₈alkoxy.

In some embodiments is a compound of Formula (IVa), wherein R₂, R₃, R₄, and R₅ are each deuterium. In some embodiments is a compound of Formula (IVa), wherein R₂, R₃, R₄, and R₅ are each hydrogen. In some embodiments is a compound of Formula (IVa), wherein R₂ is —OH, and R₃, R₄, and R₅ are each hydrogen. In some embodiments is a compound of Formula (IVa), wherein R₃ is —OH, and R₂, R₄, and R₅ are each hydrogen. In some embodiments is a compound of Formula (IVa), wherein R₂ and R₄ are each —OH, and R₃ and R₅ are each hydrogen. In some embodiments is a compound of Formula (IVa), wherein R₂ and R₅ are each —OH, and R₃ and R₄ are each hydrogen. In some embodiments is a compound of Formula (IVa), wherein R₃ and R₄ are each —OH, and R₂ and R₅ are each hydrogen. In some embodiments is a compound of Formula (IVa), wherein R₃ and R₅ are each —OH, and R₂ and R₄ are each hydrogen.

In some embodiments is a compound of Formula (IVa), wherein R₁ is substituted or unsubstituted C₁-C₈alkyl. In some embodiments is a compound of Formula (IVa), wherein R₁ is substituted C₁-C₈alkyl. In some embodiments is a compound of Formula (IVa), wherein R₁ is —CF₃. In some embodiments is a compound of Formula (IVa), wherein R₁ is unsubstituted C₁-C₈alkyl. In some embodiments is a compound of Formula (IVa), wherein R₁ is —CH₃. In some embodiments is a compound of Formula (IVa), wherein R₁ is —CH₂CH₃. In some embodiments is a compound of Formula (IVa), wherein R₁ is substituted or unsubstituted aryl. In some embodiments is a compound of Formula (IVa), wherein R₁ is unsubstituted phenyl.

In some embodiments of any one of the aspects described herein, the compound of Formula (I) is of Formula (V):

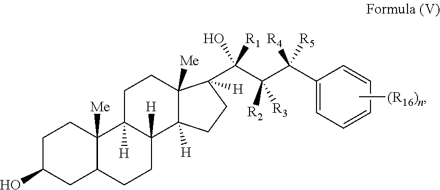

wherein:
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₈alkylaryl;
each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl;
R₂, R₃, R₄, and R₅ are independently hydrogen, deuterium, or —OH; and
n is 0, 1, 2, or 3.

In some embodiments is a compound of Formula (V), wherein n is 0. In some embodiments is a compound of Formula (V), wherein n is 1 and R₁₆ is halogen. In some embodiments is a compound of Formula (V), wherein n is 1 and R₁₆ is F. In some embodiments is a compound of Formula (V), wherein n is 1 and R₁₆ is Cl. In some embodiments is a compound of Formula (V), wherein n is 1 and R₁₆ is Br. In some embodiments is a compound of Formula (V), wherein n is 1 and R₁₆ is hydroxy. In some embodiments is a compound of Formula (V), wherein n is 1 and R₁₆ is substituted or unsubstituted C₁-C₈alkyl. In some embodiments is a compound of Formula (V), wherein n is 1 and R₁₆ is unsubstituted C₁-C₈alkyl. In some embodiments is a compound of Formula (V), wherein n is 1 and R₁₆ is substituted or unsubstituted C₁-C₈alkoxy. In some embodiments is a compound of Formula (V), wherein n is 2 and each R₁₆ is halogen. In some embodiments is a compound of Formula (V), wherein n is 2 and each R₁₆ is F. In some embodiments is a compound of Formula (V), wherein n is 2 and each R₁₆ is Cl. In some embodiments is a compound of Formula (V), wherein n is 2 and each R₁₆ is Br. In some embodiments is a compound of Formula (V), wherein n is 2 and one R₁₆ is halogen and one R₁₆ is hydroxy. In some embodiments is a compound of Formula (V), wherein n is 2 and each R₁₆ is hydroxy. In some embodiments is a compound of Formula (V), wherein n is 2 and one R₁₆ is halogen and one R₁₆ is substituted or unsubstituted C₁-C₈alkyl. In some embodiments is a compound of Formula (V), wherein n is 2 and each R₁₆ is substituted or unsubstituted C₁-C₈alkyl. In some embodiments is a compound of Formula (V), wherein ii is 2 and each R₁₆ is unsubstituted C₁-C₈alkyl. In some embodiments is a compound of Formula (V), wherein n is 2 and one R₁₆ is halogen and one R₁₆ is substituted or unsubstituted C₁-C₈alkoxy. In some embodiments is a compound of Formula (V), wherein n is 2 and each R₁₆ is substituted or unsubstituted C₁-C₈alkoxy.

In some embodiments is a compound of Formula (V), wherein R₂, R₃, R₄, and R₅ are each deuterium. In some embodiments is a compound of Formula (V), wherein R₂, R₃, R₄, and R₅ are each hydrogen. In some embodiments is a compound of Formula (V), wherein R₂ is —OH, and R₃, R₄, and R₅ are each hydrogen. In some embodiments is a compound of Formula (V), wherein R₃ is —OH, and R₂, R₄, and R₅ are each hydrogen. In some embodiments is a compound of Formula (V), wherein R₂ and R₄ are each —OH, and R₃ and R₅ are each hydrogen. In some embodiments is a compound of Formula (V), wherein R₂ and R₅ are each —OH, and R₃ and R₄ are each hydrogen. In some embodiments is a compound of Formula (V), wherein R₃ and R₄ are each —OH, and R₂ and R₅ are each hydrogen. In some embodiments is a compound of Formula (V), wherein R₃ and R₅ are each —OH, and R₂ and R₄ are each hydrogen.

In some embodiments is a compound of Formula (V), wherein R₁ is substituted or unsubstituted C₁-C₈alkyl. In some embodiments is a compound of Formula (V), wherein R₁ is substituted C₁-C₈alkyl. In some embodiments is a compound of Formula (V), wherein R₁ is —CF₃. In some embodiments is a compound of Formula (V), wherein R₁ is unsubstituted C₁-C₈alkyl. In some embodiments is a compound of Formula (V), wherein R₁ is —CH₃. In some embodiments is a compound of Formula (V), wherein R₁ is —CH₂CH₃. In some embodiments is a compound of Formula (V), wherein R₁ is substituted or unsubstituted aryl. In some embodiments is a compound of Formula (V), wherein R₁ is unsubstituted phenyl.

In some embodiments of any one of the aspects described herein, the compound of Formula (I) is of Formula (Va):

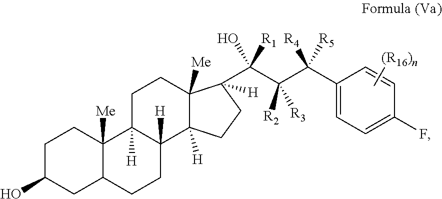

wherein:
R₁ is substituted or unsubstituted C₁-C₈alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl;
each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl;
R₂, R₃, R₄, and R₅ are independently hydrogen, deuterium, or —OH; and
n is 0, 1, or 2.

In some embodiments is a compound of Formula (Va), wherein n is 0. In some embodiments is a compound of Formula (Va), wherein n is 1 and R₁₆ is halogen. In some embodiments is a compound of Formula (Va), wherein n is 1 and R₁₆ is F. In some embodiments is a compound of Formula (Va), wherein n is 1 and R₁₆ is Cl. In some embodiments is a compound of Formula (Va), wherein n is 1 and R₁₆ is Br. In some embodiments is a compound of Formula (Va), wherein n is 1 and R₁₆ is hydroxy. In some embodiments is a compound of Formula (Va), wherein n is 1 and R₁₆ is substituted or unsubstituted C₁-C₈alkyl. In some embodiments is a compound of Formula (Va), wherein n is 1 and R₁₆ is unsubstituted C₁-C₈alkyl. In some embodiments is a compound of Formula (Va), wherein n is 1 and R₁₆ is substituted or unsubstituted C₁-C₈alkoxy. In some embodiments is a compound of Formula (Va), wherein n is 2 and each Rib is halogen. In some embodiments is a compound of Formula (Va), wherein n is 2 and each R₁₆ is F. In some embodiments is a compound of Formula (Va), wherein n is 2 and each R₁₆ is Cl. In some embodiments is a compound of Formula (Va), wherein n is 2 and each R₁₆ is Br. In some embodiments is a compound of Formula (Va), wherein n is 2 and one R₁₆ is halogen and one R₁₆ is hydroxy. In some embodiments is a compound of Formula (Va), wherein n is 2 and each R₁₆ is hydroxy. In some embodiments is a compound of Formula (Va), wherein n is 2 and one R₁₆ is halogen and one R₁₆ is substituted or unsubstituted C₁-C₈alkyl. In some embodiments is a compound of Formula (Va), wherein n is 2 and each R₁₆ is substituted or unsubstituted C₁-C₈alkyl. In some embodiments is a compound of Formula (Va), wherein n is 2 and each R₁₆ is unsubstituted C₁-C₈alkyl. In some embodiments is a compound of Formula (Va), wherein n is 2 and one R₁₆ is halogen and one R₁₆ is substituted or unsubstituted C₁-C₈alkoxy. In some embodiments is a compound of Formula (Va), wherein n is 2 and each R₁₆ is substituted or unsubstituted C₁-C₈alkoxy.

In some embodiments is a compound of Formula (Va), wherein R₂, R₃, R₄, and R₅ are each deuterium. In some embodiments is a compound of Formula (Va), wherein R₂, R₃, R₄, and R₅ are each hydrogen. In some embodiments is a compound of Formula (Va), wherein R₂ is —OH, and R₃, R₄, and R₅ are each hydrogen. In some embodiments is a compound of Formula (Va), wherein R₃ is —OH, and R₂, R₄, and R₅ are each hydrogen. In some embodiments is a compound of Formula (Va), wherein R₂ and R₄ are each —OH, and R₃ and R₅ are each hydrogen. In some embodiments is a compound of Formula (Va), wherein R₂ and R₃ are each —OH, and R₃ and R₄ are each hydrogen. In some embodiments is a compound of Formula (Va), wherein R₃ and R₄ are each —OH, and R₂ and R₅ are each hydrogen. In some embodiments is a compound of Formula (Va), wherein R₃ and R₅ are each —OH, and R₂ and R₄ are each hydrogen.

In some embodiments is a compound of Formula (Va), wherein R₁ is substituted or unsubstituted C₁-C₈alkyl. In some embodiments is a compound of Formula (Va), wherein R₁ is substituted C₁-C₈alkyl. In some embodiments is a compound of Formula (Va), wherein R₁ is-CF₃. In some embodiments is a compound of Formula (Va), wherein R₁ is unsubstituted C₁-C₈alkyl. In some embodiments is a compound of Formula (Va), wherein R₁ is —CH₃. In some embodiments is a compound of Formula (Va), wherein R₁ is —CH₂CH₃. In some embodiments is a compound of Formula (Va), wherein R₁ is substituted or unsubstituted aryl. In some embodiments is a compound of Formula (Va), wherein R₁ is unsubstituted phenyl.

In some embodiments of any one of the aspects described herein, the compound of Formula (I) is of Formula (VI):

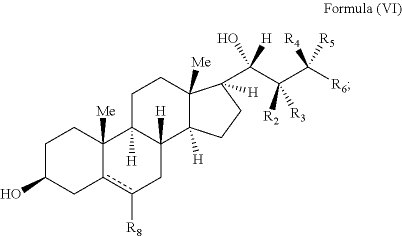

wherein:
[symbol] is a single or double bond;
R₈ is hydrogen or —OH;
R₂, R₃, R₄, and R₅ are independently hydrogen, deuterium, C₁-C₈alkyl, or —OH;
R₆ is C₆-C₁₀aryl or C₂-C₉heteroaryl, wherein C₆-C₁₀aryl or C₂-C₉heteroaryl are optionally substituted with 1, 2, 3, or 4 R₉ groups;
each R₉ is independently selected from deuterium, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₂₋₉heteroaryl, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄);
each R₁₂ is independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl;
each R₁₃ and each R₁₄ are each independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl; and
each R₁₅ is independently selected from C₁₋₆alkyl, C₁₋₆haloalkyl, C₃₋₆cycloalkyl, C₂₋₉heterocycloalkyl, C₆₋₁₀aryl, and C₁₋₉heteroaryl.

In some embodiments ofany one of the aspects described herein, the compound is of Formula (VI) wherein [symbol] is a single bond. In some embodiments of any one of the aspects described herein, the compound is of Formula (VI) wherein [symbol] is a double bond.

In some embodiments of any one of the aspects described herein, the compound is of Formula (VI), wherein R₈ is hydrogen. In some embodiments of any one of the aspects described herein, the compound is of Formula (VI), wherein R₈ is —OH.

In some embodiments of any one of the aspects described herein, the compound is of Formula (VI), wherein R₆ is C₆-C₁₀aryl optionally substituted with 1, 2, 3, or 4 R₉ groups. In some embodiments of any one of the aspects described herein, the compound is of Formula (VI), wherein R₆ is C₆-C₁₀aryl substituted with 1, 2, or 3 R₉ groups. In some embodiments of any one of the aspects described herein, the compound is of Formula (VI), wherein R₆ is phenyl substituted with 1, 2, or 3 R₉ groups. In some embodiments of any one of the aspects described herein, the compound is of Formula (VI), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is phenyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is phenyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is phenyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is phenyl substituted with 1 R₉ group and R₉ is halogen. In some embodiments of any one of the aspects described herein, the compound is of Formula (VI), wherein R₆ is phenyl substituted with 1 R₉ group and R₉ is fluoro. In some embodiments of any one of the aspects described herein, the compound is of Formula (VI), wherein R₆ is phenyl substituted with 1 R₉ group and R₉ is C₁₋₆alkyl optionally substituted with one, two, or three groups independently selected from halogen. In some embodiments of any one of the aspects described herein, the compound is of Formula (VI), wherein R₆ is phenyl substituted with 1 R₉ group and R₉ is unsubstituted C₁₋₆alkyl. In some embodiments of any one of the aspects described herein, the compound is of Formula (VI), wherein R₆ is phenyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is phenyl substituted with 1 R₉ group and R₉ is unsubstituted phenyl.

In some embodiments of any one of the aspects described herein, the compound is of Formula (VI), wherein R₆ is unsubstituted phenyl.

In some embodiments of any one of the aspects described herein, the compound is of Formula (VI), wherein R₆ is C₂-C₉heteroaryl optionally substituted with 1, 2, or 3 R₉ groups. In some embodiments of any one of the aspects described herein, the compound is of Formula (VI), wherein R₆ is phenyl substituted with 1, 2, or 3 R₉ groups. In some embodiments of any one of the aspects described herein, the compound is of Formula (VI), wherein R₆ is C₂-C₉heteroaryl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is C₂-C₉heteroaryl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is C₂-C₉heteroaryl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is C₂-C₉heteroaryl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is C₂-C₉heteroaryl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is C₂-C₉heteroaryl substituted with 1 R₉ group and R₉ is halogen. In some embodiments of any one of the aspects described herein, the compound is of Formula (VI), wherein R₆ is C₂-C₉heteroaryl substituted with 1 R₉ group and R₉ is fluoro. In some embodiments of any one of the aspects described herein, the compound is of Formula (VI), wherein R₆ is C₂-C₉heteroaryl substituted with 1 R₉ group and R₉ is C₁₋₆alkyl optionally substituted with one, two, or three groups independently selected from halogen. In some embodiments of any one of the aspects described herein, the compound is of Formula (VI), wherein R₆ is C₂-C₉heteroaryl substituted with 1 R₉ group and R₉ is unsubstituted C₁₋₆alkyl. In some embodiments of any one of the aspects described herein, the compound is of Formula (VI), wherein R₆ is C₂-C₉heteroaryl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is C₂-C₉heteroaryl substituted with 1 R₉ group and R₉ is unsubstituted phenyl.

In some embodiments of any one of the aspects described herein, the compound is of Formula (VI), wherein R₆ is unsubstituted C₂-C₉heteroaryl.

In some embodiments of any one of the aspects described herein, the compound is of Formula (VI), wherein R₆ is pyridyl optionally substituted with 1, 2, or 3 R₉ groups. In some embodiments of any one of the aspects described herein, the compound is of Formula (VI), wherein R₆ is phenyl substituted with 1, 2, or 3 R₉ groups. In some embodiments of any one of the aspects described herein, the compound is of Formula (VI), wherein R₆ is pyridyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is pyridyl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is pyridyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is pyridyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is pyridyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is pyridyl substituted with 1 R₉ group and R₉ is halogen. In some embodiments of any one of the aspects described herein, the compound is of Formula (VI), wherein R₆ is pyridyl substituted with 1 R₉ group and R₉ is fluoro. In some embodiments of any one of the aspects described herein, the compound is of Formula (VI), wherein R₆ is pyridyl substituted with 1 R₉ group and R₉ is C₁₋₆alkyl optionally substituted with one, two, or three groups independently selected from halogen. In some embodiments of any one of the aspects described herein, the compound is of Formula (VI), wherein R₆ is pyridyl substituted with 1 R₉ group and R₉ is unsubstituted C₁₋₆alkyl. In some embodiments of any one of the aspects described herein, the compound is of Formula (VI), wherein R₆ is pyridyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VI), wherein R₆ is pyridyl substituted with 1 R₉ group and R₉ is unsubstituted phenyl.

In some embodiments of any one of the aspects described herein, the compound is of Formula (VI), wherein R₆ is unsubstituted pyridyl.

In some embodiments of any one of the aspects described herein, the compound is of Formula (VI), wherein R₂, R₃, R₄, and R₅ are each hydrogen. In some embodiments of any one of the aspects described herein, the compound is of Formula (VI), wherein R₂, R₃, R₄, and R₅ are each deuterium. In some embodiments of any one of the aspects described herein, the compound is of Formula (VI), wherein R₂ is —OH, and R₃, R₄, and R₅ are each hydrogen. In some embodiments of any one of the aspects described herein, the compound is of Formula (VI), wherein R₃ is —OH, and R₂, R₄, and R₅ are each hydrogen. In some embodiments of any one of the aspects described herein, the compound is of Formula (VI), wherein R₂ and R₄ are each —OH, and R₃ and R₅ are each hydrogen. In some embodiments of any one of the aspects described herein, the compound is of Formula (VI), wherein R₂ and R₅ are each —OH, and R₃ and R₄ are each hydrogen. In some embodiments of any one of the aspects described herein, the compound is of Formula (VI), wherein R₃ and R₄ are each —OH, and R₂ and R₅ are each hydrogen. In some embodiments of any one of the aspects described herein, the compound is of Formula (VI), wherein R₃ and R₅ are each —OH, and R₂ and R₄ are each hydrogen.

In some embodiments of any one of the aspects described herein, the compound of Formula (I) is of Formula (VIa):

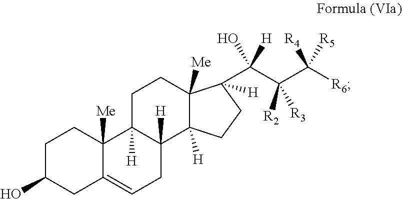

wherein:
R₂, R₃, R₄, and R₅ are independently hydrogen, deuterium, C₁-C₈alkyl, or —OH;
R₆ is C₆-C₁₀aryl or C₂-C₉heteroaryl, wherein C₆-C₁₀aryl or C₂-C₉heteroaryl are optionally substituted with 1, 2, 3, or 4 R₉ groups;
   each R₉ is independently selected from deuterium, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₂₋₉heteroaryl, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄);
   each R₁₂ is independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl;
   each R₁₃ and each R₁₄ are each independently selected from H, C₁₋₆alkyl, C₁₋₆haloalkyl, C₃₋₆cycloalkyl, C₂₋₉heterocycloalkyl, C₆₋₁₀aryl, and C₁₋₆heteroaryl; and
   each R₁₅ is independently selected from C₁₋₆alkyl, C₁₋₆haloalkyl, C₃₋₆cycloalkyl, C₂₋₉heterocycloalkyl, C₆₋₁₀aryl, and C₁₋₉heteroaryl.

In some embodiments of any one of the aspects described herein, the compound is of Formula (VIa), wherein R₆ is C₆-C₁₀aryl optionally substituted with 1, 2, 3, or 4 R₉ groups. In some embodiments of any one of the aspects described herein, the compound is of Formula (VIa), wherein R₆ is C₆-C₁₀aryl substituted with 1, 2, or 3 R₉ groups. In some embodiments of any one of the aspects described herein, the compound is of Formula (VIa), wherein R₆ is phenyl substituted with 1, 2, or 3 R₉ groups. In some embodiments of any one of the aspects described herein, the compound is of Formula (VIa), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is phenyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is phenyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is phenyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is phenyl substituted with 1 R₉ group and R₉ is halogen. In some embodiments of any one of the aspects described herein, the compound is of Formula (VIa), wherein R₆ is phenyl substituted with 1 R₉ group and R₉ is fluoro. In some embodiments of any one of the aspects described herein, the compound is of Formula (VIa), wherein R₆ is phenyl substituted with 1 R₉ group and R₉ is C₁₋₆alkyl optionally substituted with one, two, or three groups independently selected from halogen. In some embodiments of any one of the aspects described herein, the compound is of Formula (VIa), wherein R₆ is phenyl substituted with 1 R₉ group and R₉ is unsubstituted C₁₋₆alkyl. In some embodiments of any one of the aspects described herein, the compound is of Formula (VIa), wherein R₆ is phenyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is phenyl substituted with 1 R₉ group and R₉ is unsubstituted phenyl.

In some embodiments of any one of the aspects described herein, the compound is of Formula (VIa), wherein R₆ is unsubstituted phenyl.

In some embodiments of any one of the aspects described herein, the compound is of Formula (VIa), wherein R₆ is C₂-C₉heteroaryl optionally substituted with 1, 2, or 3 R₉ groups. In some embodiments of any one of the aspects described herein, the compound is of Formula (VIa), wherein R₆ is phenyl substituted with 1, 2, or 3 R₉ groups. In some embodiments of any one of the aspects described herein, the compound is of Formula (VIa), wherein R₆ is C₂-C₉heteroaryl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is C₂-C₉heteroaryl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is C₂-C₉heteroaryl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is C₂-C₉heteroaryl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is C₂-C₉heteroaryl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is C₂-C₉heteroaryl substituted with 1 R₉ group and R₉ is halogen. In some embodiments of any one of the aspects described herein, the compound is of Formula (VIa), wherein R₆ is C₂-C₉heteroaryl substituted with 1 R₉ group and R₉ is fluoro. In some embodiments of any one of the aspects described herein, the compound is of Formula (VIa), wherein R₆ is C₂-C₉heteroaryl substituted with 1 R₉ group and R₉ is C₁₋₆alkyl optionally substituted with one, two, or three groups independently selected from halogen. In some embodiments of any one of the aspects described herein, the compound is of Formula (VIa), wherein R₆ is C₂-C₉heteroaryl substituted with 1 R₉ group and R₉ is unsubstituted C₁₋₆alkyl. In some embodiments of any one of the aspects described herein, the compound is of Formula (VIa), wherein R₆ is C₂-C₉heteroaryl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is C₂-C₉heteroaryl substituted with 1 R₉ group and R₉ is unsubstituted phenyl.

In some embodiments of any one of the aspects described herein, the compound is of Formula (VIa), wherein R₆ is unsubstituted C₂-C₉heteroaryl.

In some embodiments of any one of the aspects described herein, the compound is of Formula (VIa), wherein R₆ is pyridyl optionally substituted with 1, 2, or 3 R₉ groups. In some embodiments of any one of the aspects described herein, the compound is of Formula (VIa), wherein R₆ is phenyl substituted with 1, 2, or 3 R₉ groups. In some embodiments of any one of the aspects described herein, the compound is of Formula (VIa), wherein R₆ is pyridyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is pyridyl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is pyridyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is pyridyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is pyridyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is pyridyl substituted with 1 R₉ group and R₉ is halogen. In some embodiments of any one of the aspects described herein, the compound is of Formula (VIa), wherein R₆ is pyridyl substituted with 1 R₉ group and R₉ is fluoro. In some embodiments of any one of the aspects described herein, the compound is of Formula (VIa), wherein R₆ is pyridyl substituted with 1 R₉ group and R₉ is C₁₋₆alkyl optionally substituted with one, two, or three groups independently selected from halogen. In some embodiments of any one of the aspects described herein, the compound is of Formula (VIa), wherein R₆ is pyridyl substituted with 1 R₉ group and R₉ is unsubstituted C₁₋₆alkyl. In some embodiments of any one of the aspects described herein, the compound is of Formula (VIa), wherein R₆ is pyridyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIa), wherein R₆ is pyridyl substituted with 1 R₉ group and R₉ is unsubstituted phenyl.

In some embodiments of any one of the aspects described herein, the compound is of Formula (VIa), wherein R₆ is unsubstituted pyridyl.

In some embodiments of any one of the aspects described herein, the compound is of Formula (VIa), wherein R₂, R₃, R₄, and R₅ are each hydrogen. In some embodiments of any one of the aspects described herein, the compound is of Formula (VIa), wherein R₂, R₃, R₄, and R₅ are each deuterium. In some embodiments of any one of the aspects described herein, the compound is of Formula (VIa), wherein R₂ is —OH, and R₃, R₄, and R₅ are each hydrogen. In some embodiments of any one of the aspects described herein, the compound is of Formula (VIa), wherein R₃ is —OH, and R₂, R₄, and R₅ are each hydrogen. In some embodiments of any one of the aspects described herein, the compound is of Formula (VIa), wherein R₂ and R₄ are each —OH, and R₃ and R₅ are each hydrogen. In some embodiments of any one of the aspects described herein, the compound is of Formula (VIa), wherein R₂ and R₅ are each —OH, and R₃ and R₄ are each hydrogen. In some embodiments of any one of the aspects described herein, the compound is of Formula (VIa), wherein R₃ and R₄ are each —OH, and R₂ and R₅ are each hydrogen. In some embodiments of any one of the aspects described herein, the compound is of Formula (VIa), wherein R₃ and R₅ are each —OH, and R₂ and R₄ are each hydrogen.

In some embodiments of any one of the aspects described herein, the compound of Formula (I) is of Formula (VIb):

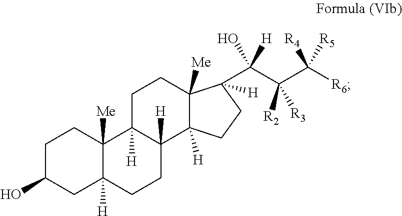

wherein:
R₂, R₃, R₄, and R₅ are independently hydrogen, deuterium, C₁-C₈alkyl, or —OH;
R₆ is C₆-C₁₀aryl or C₂-C₉heteroaryl, wherein C₆-C₁₀aryl or C₂-C₉heteroaryl are optionally substituted with 1, 2, 3, or 4 R₉ groups;
   each R₉ is independently selected from deuterium, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₂₋₉heteroaryl, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄);
   each R₁₂ is independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl;
   each R₁₃ and each R₁₄ are each independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl; and
   each R₁₅ is independently selected from C₁₋₆alkyl, C₁₋₆haloalkyl, C₃₋₆cycloalkyl, C₂₋₉heterocycloalkyl, C₆₋₁₀aryl, and C₁₋₉heteroaryl.

In some embodiments of any one of the aspects described herein, the compound is of Formula (VIb), wherein R₆ is C₆-C₁₀aryl optionally substituted with 1, 2, 3, or 4 R₉ groups. In some embodiments of any one of the aspects described herein, the compound is of Formula (VIb), wherein R₆ is C₆-C₁₀aryl substituted with 1, 2, or 3 R₉ groups. In some embodiments of any one of the aspects described herein, the compound is of Formula (VIb), wherein R₆ is phenyl substituted with 1, 2, or 3 R₉ groups. In some embodiments of any one of the aspects described herein, the compound is of Formula (VIb), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is phenyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is phenyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is phenyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is phenyl substituted with 1 R₉ group and R₉ is halogen. In some embodiments of any one of the aspects described herein, the compound is of Formula (VIb), wherein R₆ is phenyl substituted with 1 R₉ group and R₉ is fluoro. In some embodiments of any one of the aspects described herein, the compound is of Formula (VIb), wherein R₆ is phenyl substituted with 1 R₉ group and R₉ is C₁₋₆alkyl optionally substituted with one, two, or three groups independently selected from halogen. In some embodiments of any one of the aspects described herein, the compound is of Formula (VIb), wherein R₆ is phenyl substituted with 1 R₉ group and R₉ is unsubstituted C₁₋₆alkyl. In some embodiments of any one of the aspects described herein, the compound is of Formula (VIb), wherein R₆ is phenyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is phenyl substituted with 1 R₉ group and R₉ is unsubstituted phenyl.

In some embodiments of any one of the aspects described herein, the compound is of Formula (VIb), wherein R₆ is unsubstituted phenyl.

In some embodiments of any one of the aspects described herein, the compound is of Formula (VIb), wherein R₆ is C₂-C₉heteroaryl optionally substituted with 1, 2, or 3 R₉ groups. In some embodiments of any one of the aspects described herein, the compound is of Formula (VIb), wherein R₆ is phenyl substituted with 1, 2, or 3 R₉ groups. In some embodiments of any one of the aspects described herein, the compound is of Formula (VIb), wherein R₆ is C₂-C₉heteroaryl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is C₂-C₉heteroaryl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is C₂-C₉heteroaryl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is C₂-C₉heteroaryl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is C₂-C₉heteroaryl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is C₂-C₉heteroaryl substituted with 1 R₉ group and R₉ is halogen. In some embodiments of any one of the aspects described herein, the compound is of Formula (VIb), wherein R₆ is C₂-C₉heteroaryl substituted with 1 R₉ group and R₉ is fluoro. In some embodiments of any one of the aspects described herein, the compound is of Formula (VIb), wherein R₆ is C₂-C₉heteroaryl substituted with 1 R₉ group and R₉ is C₁₋₆alkyl optionally substituted with one, two, or three groups independently selected from halogen. In some embodiments of any one of the aspects described herein, the compound is of Formula (VIb), wherein R₆ is C₂-C₉heteroaryl substituted with 1 R₉ group and R₉ is unsubstituted C₁₋₆alkyl. In some embodiments of any one of the aspects described herein, the compound is of Formula (VIb), wherein R₆ is C₂-C₉heteroaryl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is C₂-C₉heteroaryl substituted with 1 R₉ group and R₉ is unsubstituted phenyl.

In some embodiments of any one of the aspects described herein, the compound is of Formula (VIb), wherein R₆ is unsubstituted C₂-C₉heteroaryl.

In some embodiments of any one of the aspects described herein, the compound is of Formula (VIb), wherein R₆ is pyridyl optionally substituted with 1, 2, or 3 R₉ groups. In some embodiments of any one of the aspects described herein, the compound is of Formula (VIb), wherein R₆ is phenyl substituted with 1, 2, or 3 R₉ groups. In some embodiments of any one of the aspects described herein, the compound is of Formula (VIb), wherein R₆ is pyridyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is pyridyl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is pyridyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is pyridyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is pyridyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is pyridyl substituted with 1 R₉ group and R₉ is halogen. In some embodiments of any one of the aspects described herein, the compound is of Formula (VIb), wherein R₆ is pyridyl substituted with 1 R₉ group and R₉ is fluoro. In some embodiments of any one of the aspects described herein, the compound is of Formula (VIb), wherein R₆ is pyridyl substituted with 1 R₉ group and R₉ is C₁₋₆alkyl optionally substituted with one, two, or three groups independently selected from halogen. In some embodiments of any one of the aspects described herein, the compound is of Formula (VIb), wherein R₆ is pyridyl substituted with 1 R₉ group and R₉ is unsubstituted C₁₋₆alkyl. In some embodiments of any one of the aspects described herein, the compound is of Formula (VIb), wherein R₆ is pyridyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIb), wherein R₆ is pyridyl substituted with 1 R₉ group and R₉ is unsubstituted phenyl.

In some embodiments of any one of the aspects described herein, the compound is of Formula (VIb), wherein R₆ is unsubstituted pyridyl.

In some embodiments of any one of the aspects described herein, the compound is of Formula (VIb), wherein R₂, R₃, R₄, and R₅ are each hydrogen. In some embodiments of any one of the aspects described herein, the compound is of Formula (VIb), wherein R₂, R₃, R₄, and R₅ are each deuterium. In some embodiments of any one of the aspects described herein, the compound is of Formula (VIb), wherein R₂ is —OH, and R₃, R₄, and R₅ are each hydrogen. In some embodiments of any one of the aspects described herein, the compound is of Formula (VIb), wherein R₃ is —OH, and R₂, R₄, and R₅ are each hydrogen. In some embodiments of any one of the aspects described herein, the compound is of Formula (VIb), wherein R₂ and R₄ are each —OH, and R₃ and R₅ are each hydrogen. In some embodiments of any one of the aspects described herein, the compound is of Formula (VIb), wherein R₂ and R₅ are each —OH, and R₃ and R₄ are each hydrogen. In some embodiments of any one of the aspects described herein, the compound is of Formula (VIb), wherein R₃ and R₄ are each —OH, and R₂ and R₅ are each hydrogen. In some embodiments of any one of the aspects described herein, the compound is of Formula (VIb), wherein R₃ and R₅ are each —OH, and R₂ and R₄ are each hydrogen.

In some embodiments of any one of the aspects described herein, the compound of Formula (I) is of Formula (VIc):

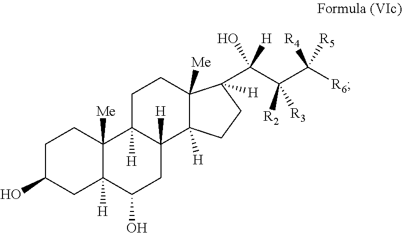

wherein:
R₂, R₃, R₄, and R₅ are independently hydrogen, deuterium, C₁-C₈alkyl, or —OH;
R₆ is C₆-C₁₀aryl or C₂-C₉heteroaryl, wherein C₆-C₁₀aryl or C₂-C₉heteroaryl are optionally substituted with 1, 2, 3, or 4 R₉ groups;
   each R₉ is independently selected from deuterium, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₂₋₉heteroaryl, —OR₁₂, —SR₁₂, —N(R₁₃)(R₁₄), —C(O)OR₁₅, —C(O)N(R₁₃)(R₁₄), —C(O)R₁₅, —S(O)₂R₁₅, and —S(O)₂N(R₁₃)(R₁₄), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR₁₂, —SR₁₂, —N(R₁₃)(R₁₄), —C(O)OR₁₅, —C(O)N(R₁₃)(R₁₄), —C(O)R₁₅, —S(O)₂R₁₅, and —S(O)₂N(R₁₃)(R₁₄);
   each R₁₂ is independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl;
   each R₁₃ and each R₁₄ are each independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl; and
   each R₁₅ is independently selected from C₁₋₆alkyl, C₁₋₆haloalkyl, C₃₋₆cycloalkyl, C₂₋₉heterocycloalkyl, C₆₋₁₀aryl, and C₁₋₉heteroaryl.

In some embodiments of any one of the aspects described herein, the compound is of Formula (VIc), wherein R₆ is C₆-C₁₀aryl optionally substituted with 1, 2, 3, or 4 R₉ groups. In some embodiments of any one of the aspects described herein, the compound is of Formula (VIc), wherein R₆ is C₆-C₁₀aryl substituted with 1, 2, or 3 R₉ groups. In some embodiments of any one of the aspects described herein, the compound is of Formula (VIc), wherein R₆ is phenyl substituted with 1, 2, or 3 R₉ groups. In some embodiments of any one of the aspects described herein, the compound is of Formula (VIc), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is phenyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is phenyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is phenyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is phenyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is phenyl substituted with 1 R₉ group and R₉ is halogen. In some embodiments of any one of the aspects described herein, the compound is of Formula (VIc), wherein R₆ is phenyl substituted with 1 R₉ group and R₉ is fluoro. In some embodiments of any one of the aspects described herein, the compound is of Formula (VIc), wherein R₆ is phenyl substituted with 1 R₉ group and R₉ is C₁₋₆alkyl optionally substituted with one, two, or three groups independently selected from halogen. In some embodiments of any one of the aspects described herein, the compound is of Formula (VIc), wherein R₆ is phenyl substituted with 1 R₉ group and R₉ is unsubstituted C₁₋₆alkyl. In some embodiments of any one of the aspects described herein, the compound is of Formula (VIc), wherein R₆ is phenyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is phenyl substituted with 1 R₉ group and R₉ is unsubstituted phenyl.

In some embodiments of any one of the aspects described herein, the compound is of Formula (VIc), wherein R₆ is unsubstituted phenyl.

In some embodiments of any one of the aspects described herein, the compound is of Formula (VIc), wherein R₆ is C₂-C₉heteroaryl optionally substituted with 1, 2, or 3 R₉ groups. In some embodiments of any one of the aspects described herein, the compound is of Formula (VIc), wherein R₆ is phenyl substituted with 1, 2, or 3 R₉ groups. In some embodiments of any one of the aspects described herein, the compound is of Formula (VIc), wherein R₆ is C₂-C₉heteroaryl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is C₂-C₉heteroaryl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is C₂-C₉heteroaryl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is C₂-C₉heteroaryl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is C₂-C₉heteroaryl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is C₂-C₉heteroaryl substituted with 1 R₉ group and R₉ is halogen. In some embodiments of any one of the aspects described herein, the compound is of Formula (VIc), wherein R₆ is C₂-C₉heteroaryl substituted with 1 R₉ group and R₉ is fluoro. In some embodiments of any one of the aspects described herein, the compound is of Formula (VIc), wherein R₆ is C₂-C₉heteroaryl substituted with 1 R₉ group and R₉ is C₁₋₆alkyl optionally substituted with one, two, or three groups independently selected from halogen. In some embodiments of any one of the aspects described herein, the compound is of Formula (VIc), wherein R₆ is C₂-C₉heteroaryl substituted with 1 R₉ group and R₉ is unsubstituted C₁₋₆alkyl. In some embodiments of any one of the aspects described herein, the compound is of Formula (VIc), wherein R₆ is C₂-C₉heteroaryl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is C₂-C₉heteroaryl substituted with 1 R₉ group and R₉ is unsubstituted phenyl.

In some embodiments of any one of the aspects described herein, the compound is of Formula (VIc), wherein R₆ is unsubstituted C₂-C₉heteroaryl.

In some embodiments of any one of the aspects described herein, the compound is of Formula (VIc), wherein R₆ is pyridyl optionally substituted with 1, 2, or 3 R₉ groups. In some embodiments of any one of the aspects described herein, the compound is of Formula (VIc), wherein R₆ is phenyl substituted with 1, 2, or 3 R₉ groups. In some embodiments of any one of the aspects described herein, the compound is of Formula (VIc), wherein R₆ is pyridyl substituted with 1, 2, or 3 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is pyridyl substituted with 1 or 2 R₉ groups and each R₉ is independently selected from halogen, C₁₋₆alkyl, C₆₋₁₀aryl, and C₂₋₉heteroaryl, wherein C₁₋₆alkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is pyridyl substituted with 1 or 2 R₉ groups and each R₉ is independently selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is pyridyl substituted with 1 R₉ group and R₉ is selected from halogen, C₁₋₆alkyl, phenyl, and C₂₋₉heteroaryl, wherein C₁₋₆alkyl, phenyl, and C₂₋₉heteroaryl are optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is pyridyl substituted with 1 R₉ group and R₉ is selected from halogen, C₁₋₆alkyl, and phenyl, wherein C₁₋₆alkyl and phenyl is optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is pyridyl substituted with 1 R₉ group and R₉ is halogen. In some embodiments of any one of the aspects described herein, the compound is of Formula (VIc), wherein R₆ is pyridyl substituted with 1 R₉ group and R₉ is fluoro. In some embodiments of any one of the aspects described herein, the compound is of Formula (VIc), wherein R₆ is pyridyl substituted with 1 R₉ group and R₉ is C₁₋₆alkyl optionally substituted with one, two, or three groups independently selected from halogen. In some embodiments of any one of the aspects described herein, the compound is of Formula (VIc), wherein R₆ is pyridyl substituted with 1 R₉ group and R₉ is unsubstituted C₁₄alkyl. In some embodiments of any one of the aspects described herein, the compound is of Formula (VIc), wherein R₆ is pyridyl substituted with 1 R₉ group and R₉ is phenyl optionally substituted with one, two, or three groups independently selected from halogen, C₁₋₆alkyl, C₁₋₆haloalkyl, C₁₋₆alkoxy, and C₁₋₆haloalkoxy. In some embodiments of any one of the aspects described herein, the compound is of Formula (VIc), wherein R₆ is pyridyl substituted with 1 R₉ group and R₉ is unsubstituted phenyl.

In some embodiments of any one of the aspects described herein, the compound is of Formula (VIc), wherein R₆ is unsubstituted pyridyl.

In some embodiments of any one of the aspects described herein, the compound is of Formula (VIc), wherein R₂, R₃, R₄, and R₅ are each hydrogen. In some embodiments of any one of the aspects described herein, the compound is of Formula (VIc), wherein R₂, R₃, R₄, and R₅ are each deuterium. In some embodiments of any one of the aspects described herein, the compound is of Formula (VIc), wherein R₂ is —OH, and R₃, R₄, and R₅ are each hydrogen. In some embodiments of any one of the aspects described herein, the compound is of Formula (VIc), wherein R₃ is —OH, and R₂, R₄, and R₅ are each hydrogen. In some embodiments of any one of the aspects described herein, the compound is of Formula (VIc), wherein R₂ and R₄ are each —OH, and R₃ and R₅ are each hydrogen. In some embodiments of any one of the aspects described herein, the compound is of Formula (VIc), wherein R₂ and R₅ are each —OH, and R₃ and R₄ are each hydrogen. In some embodiments of any one of the aspects described herein, the compound is of Formula (VIc), wherein R₃ and R₄ are each —OH, and R₂ and R₅ are each hydrogen. In some embodiments of any one of the aspects described herein, the compound is of Formula (VIc), wherein R₃ and R₅ are each —OH, and R₂ and R₄ are each hydrogen.

In some embodiments of any one of the aspects described herein, the compound of Formula (I) is of Formula (VII):

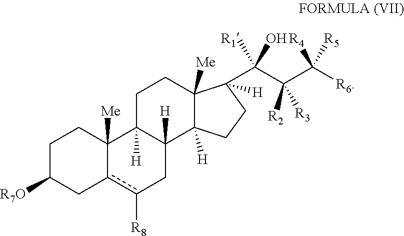

In some embodiments of any one of the aspects described herein, the compound of Formula (I) is of Formula (VIIa):

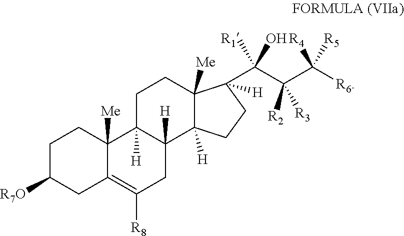

In some embodiments of any one of the aspects described herein, the compound of Formula (I) is of Formula (VIIb):

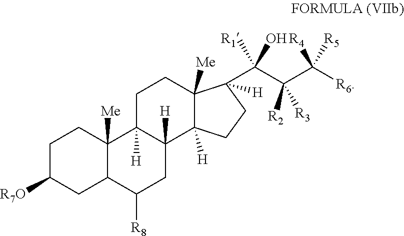

In some embodiments of the various aspect described herein, the compound is selected from the following:

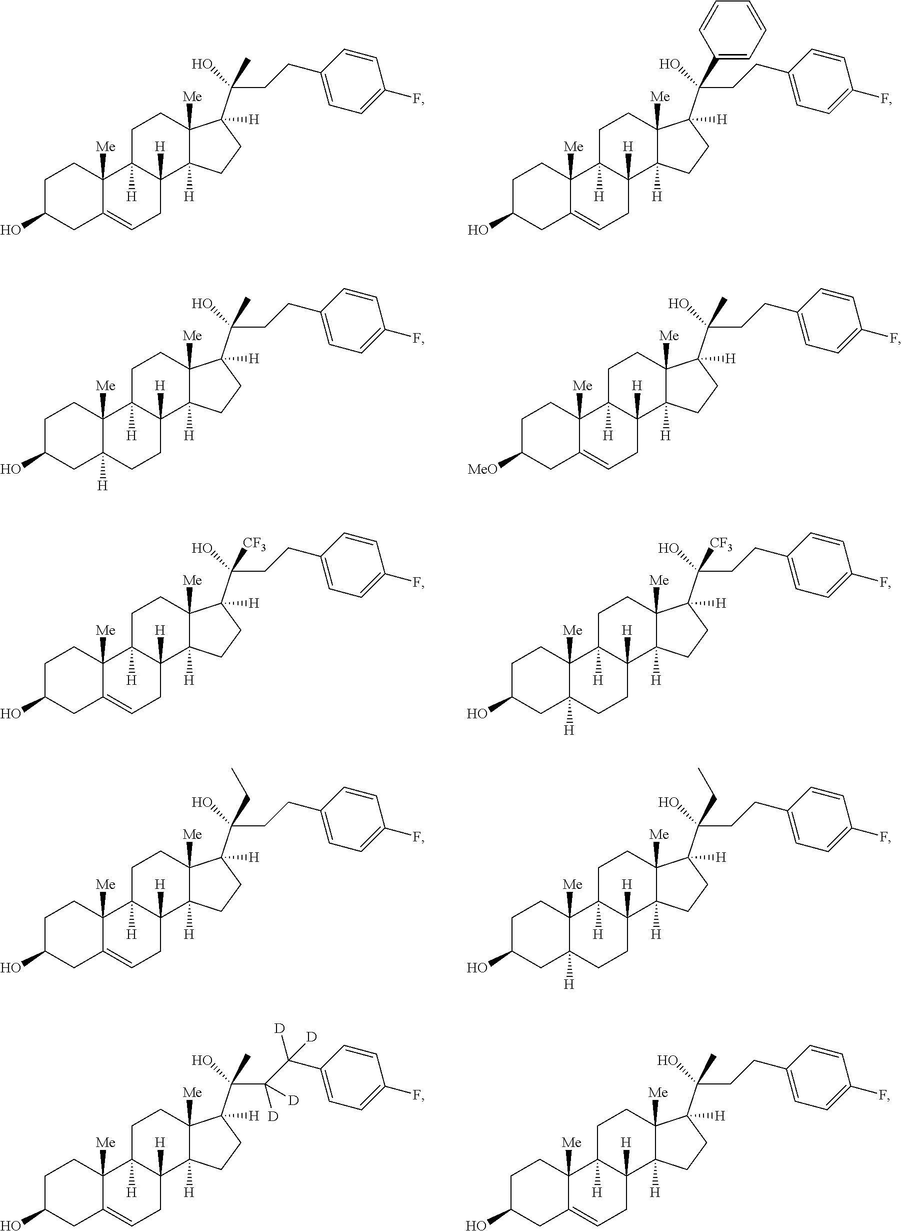

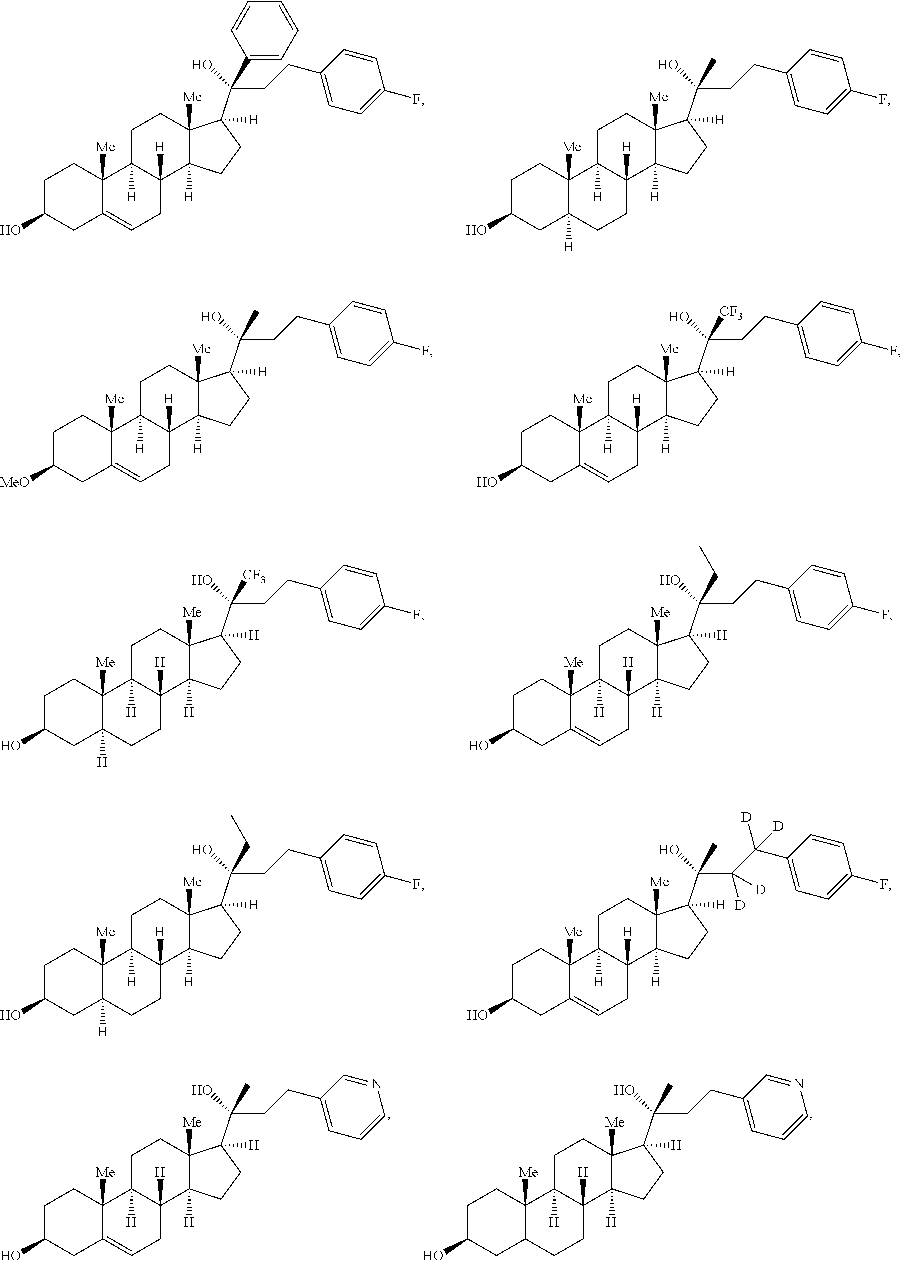

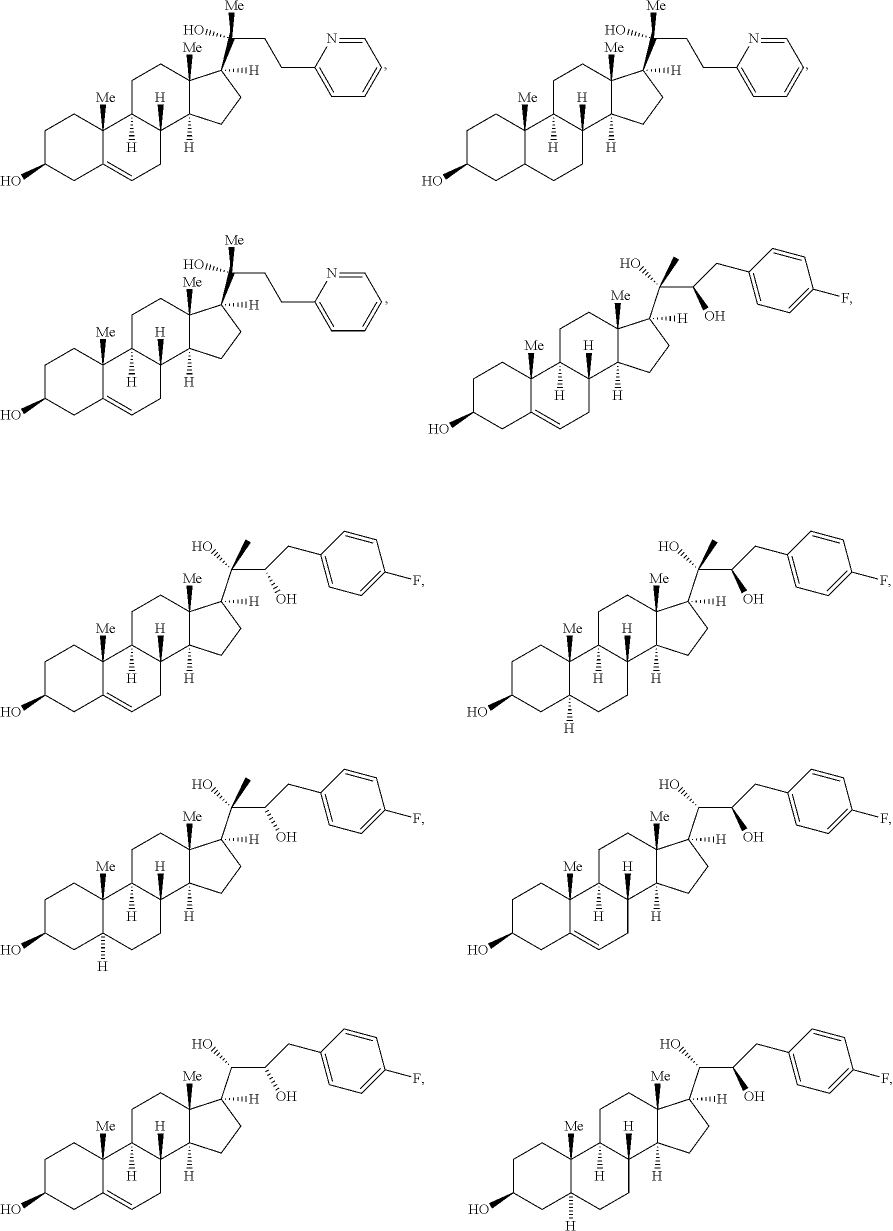

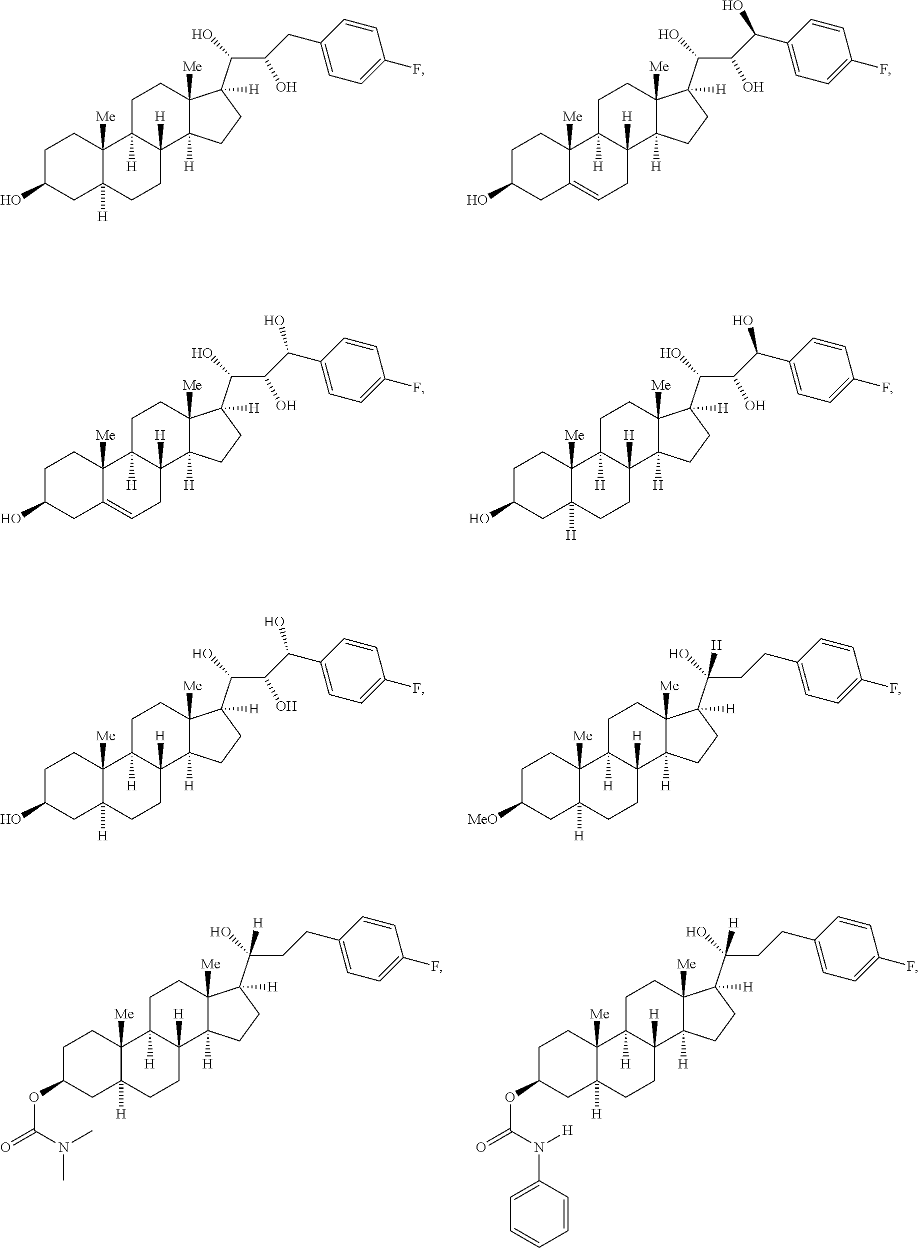

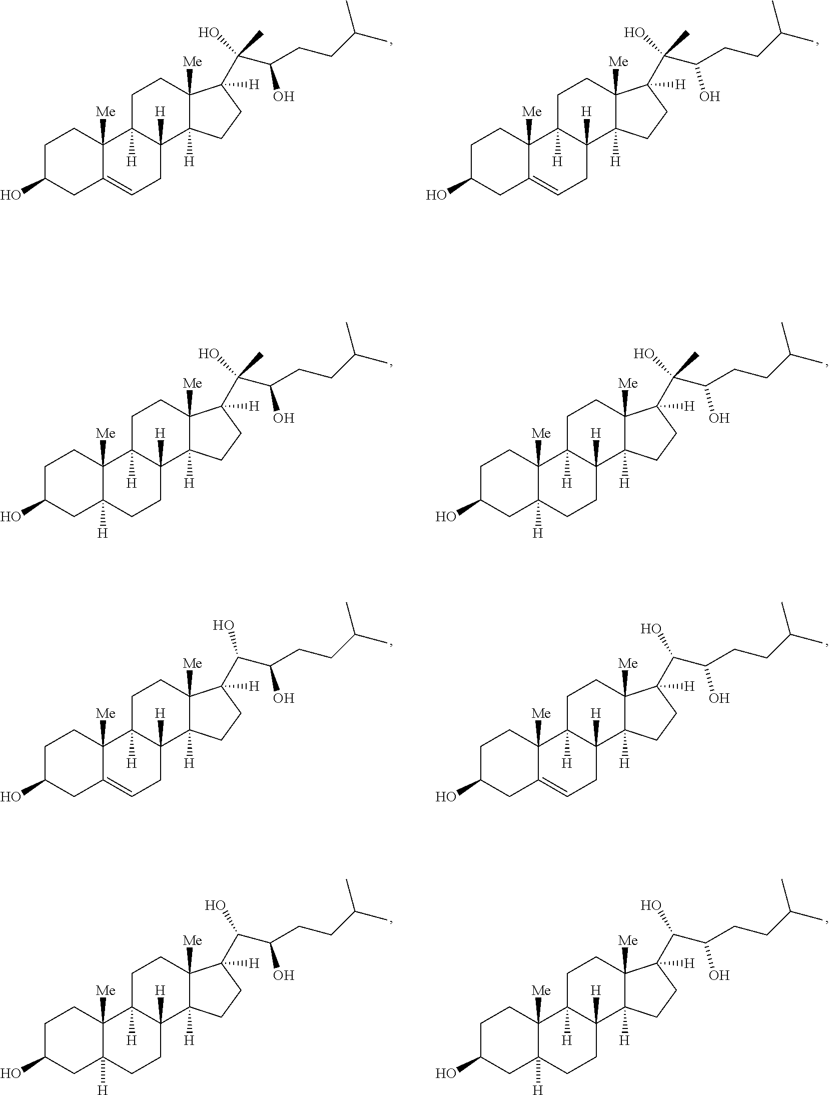

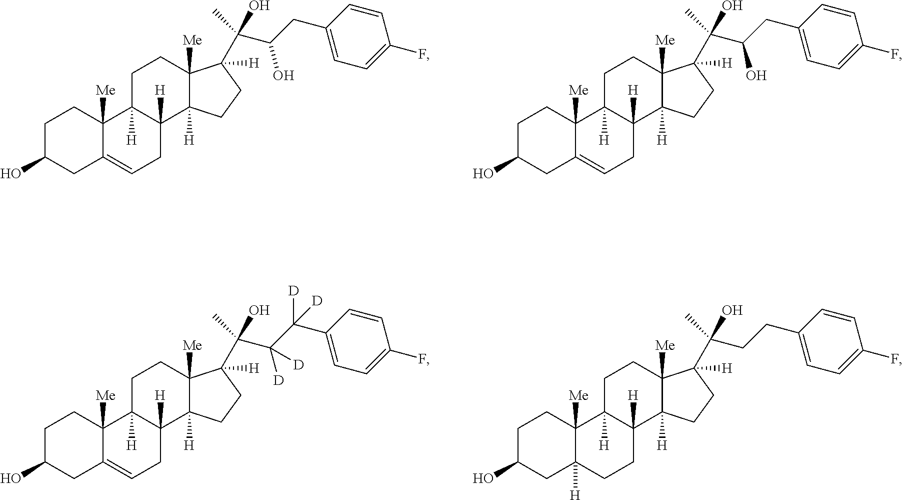

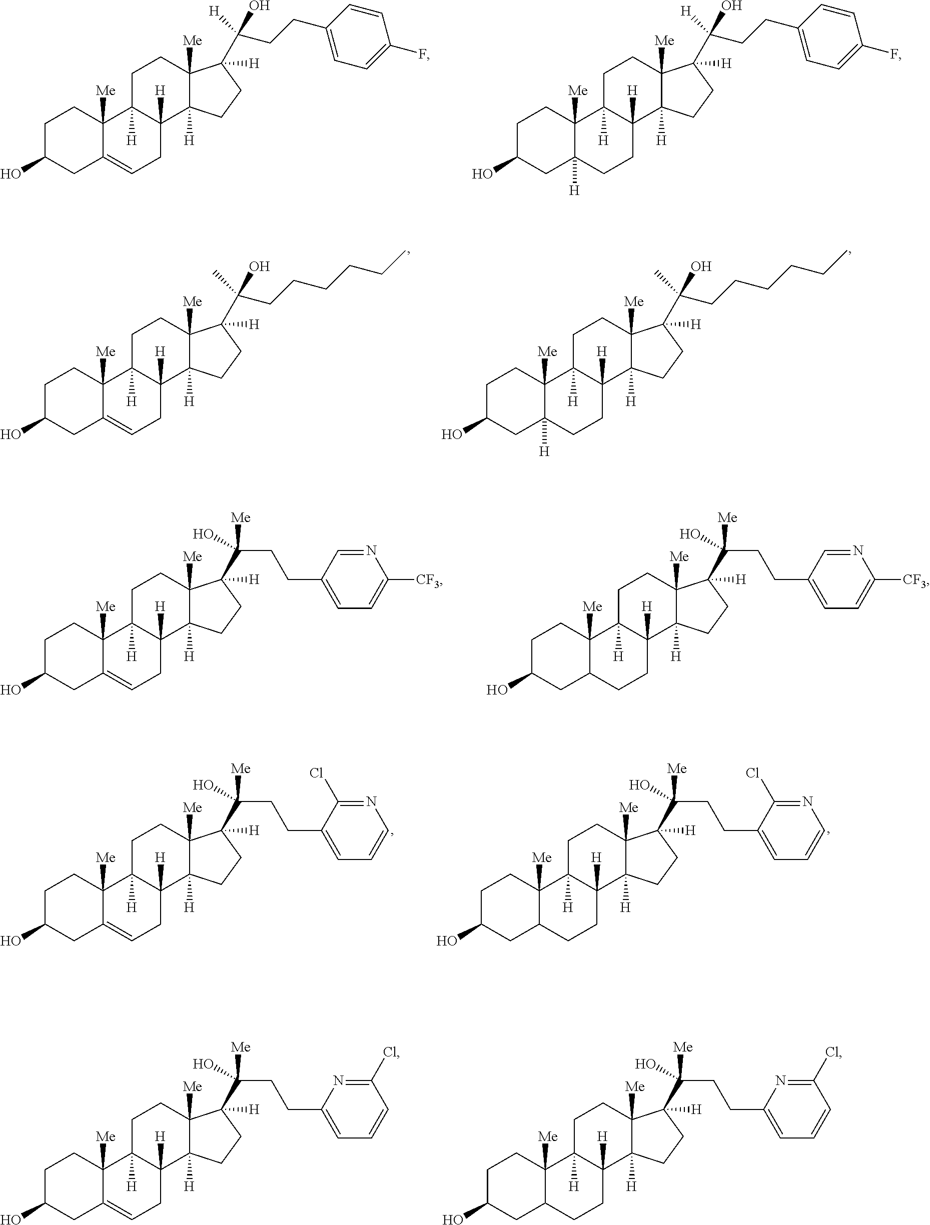

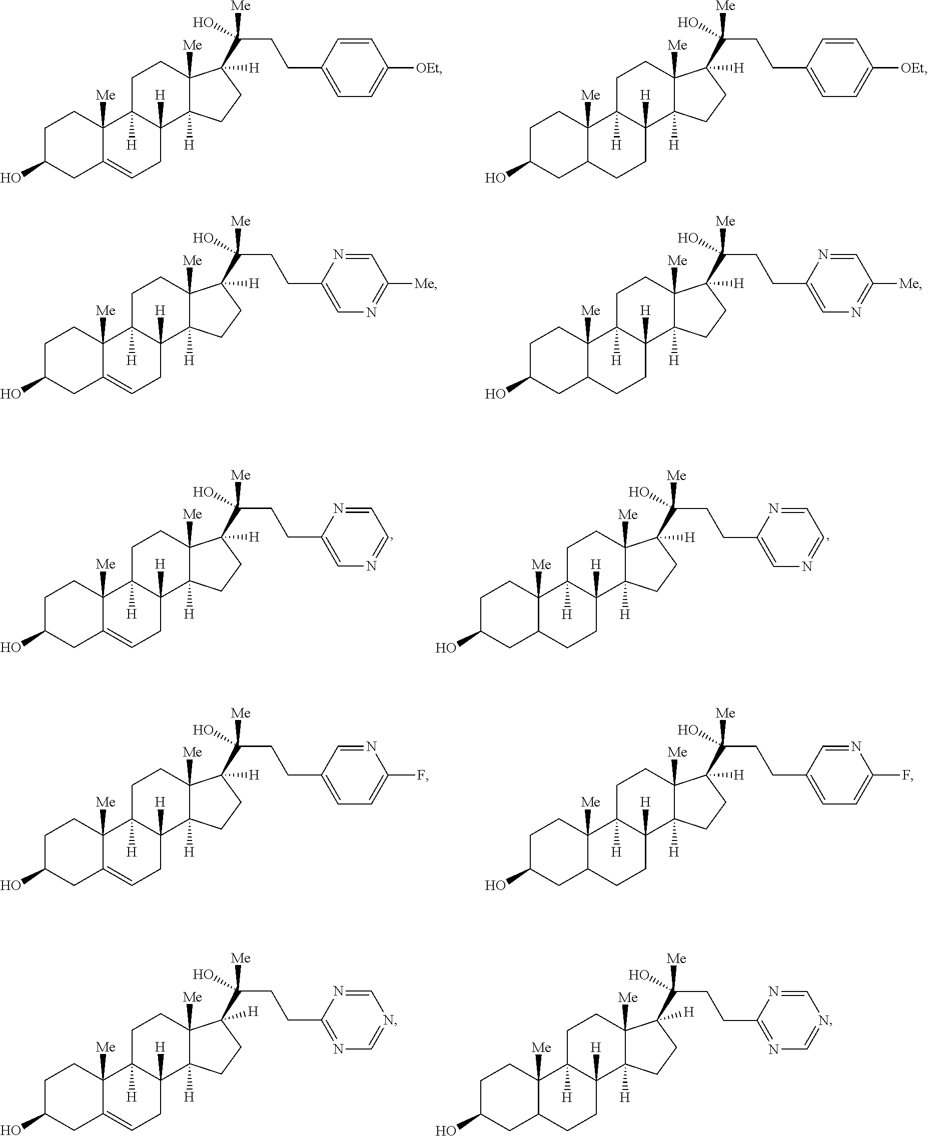

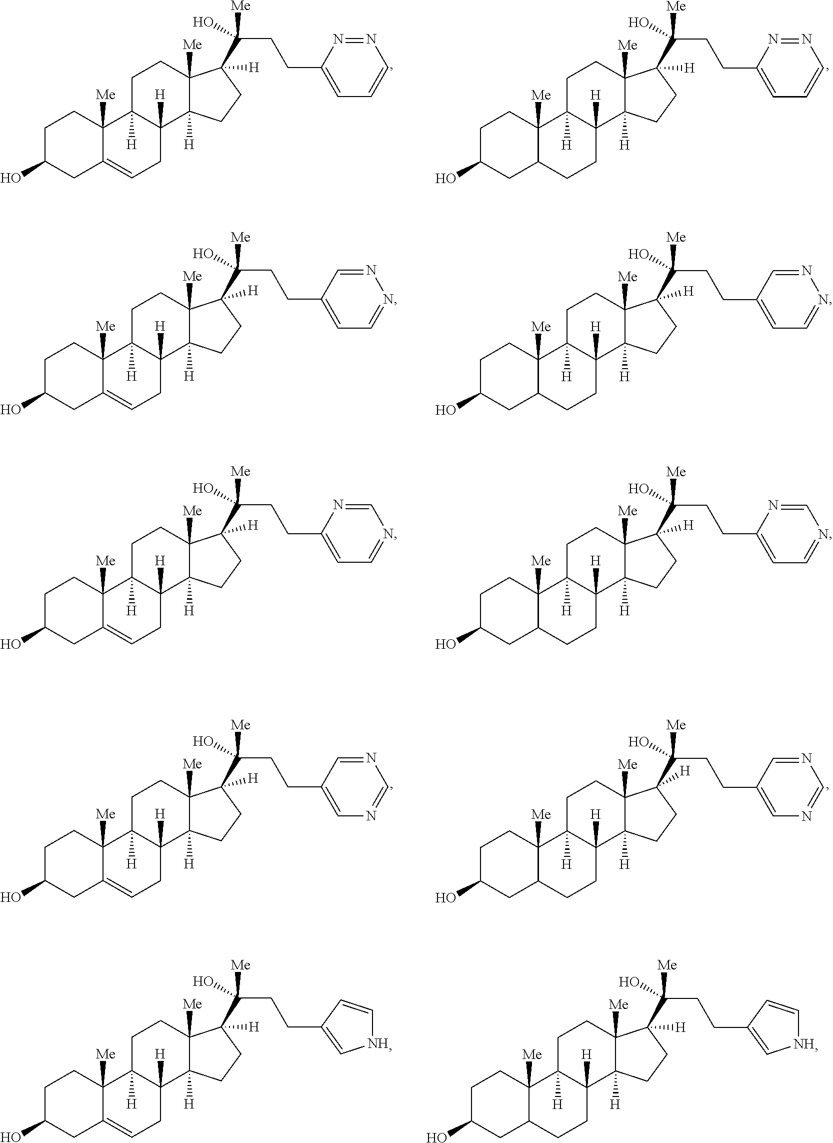

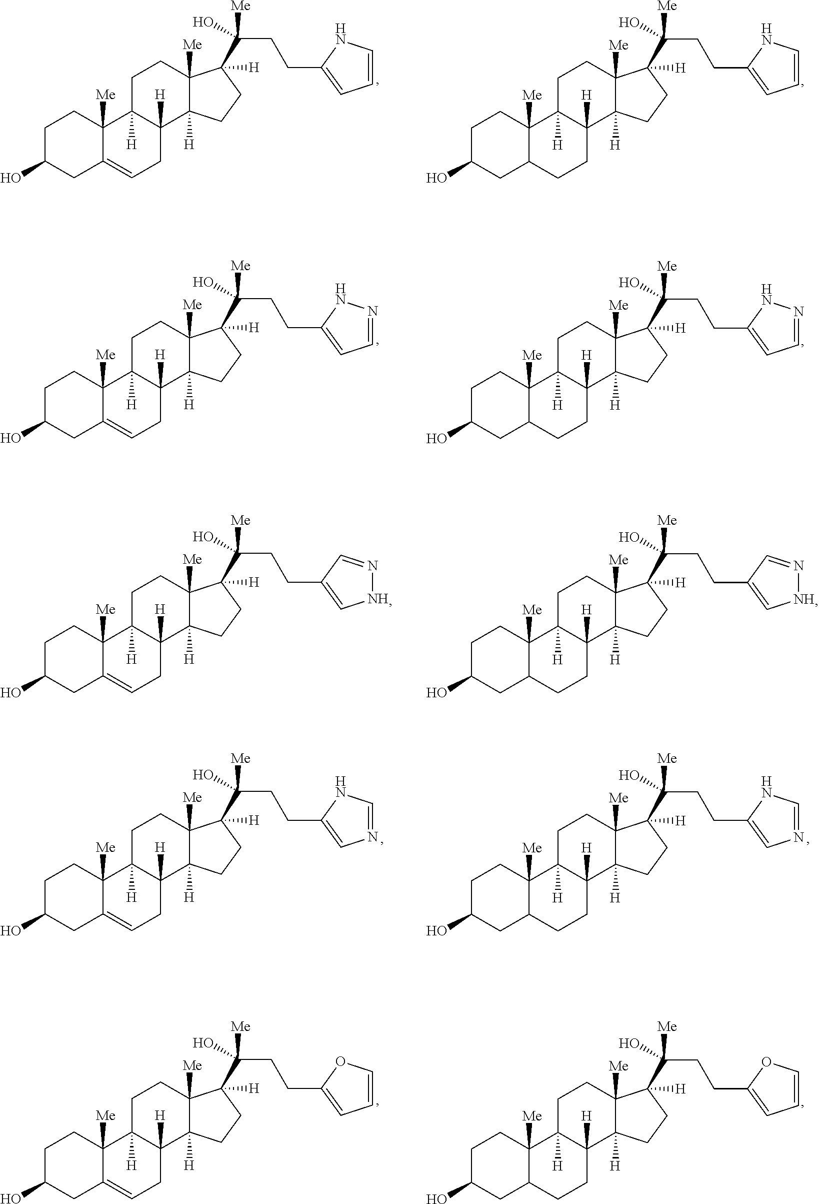

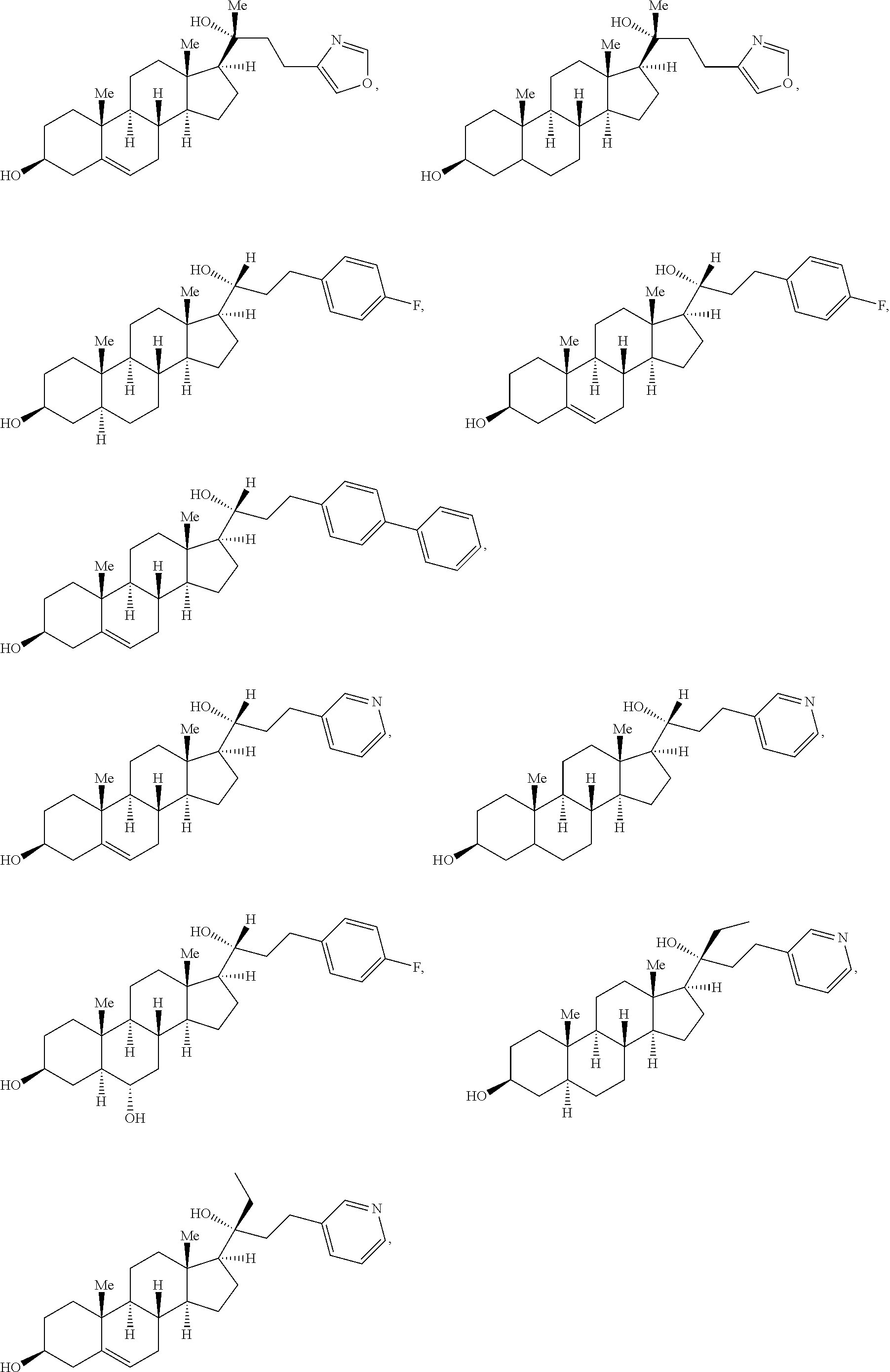

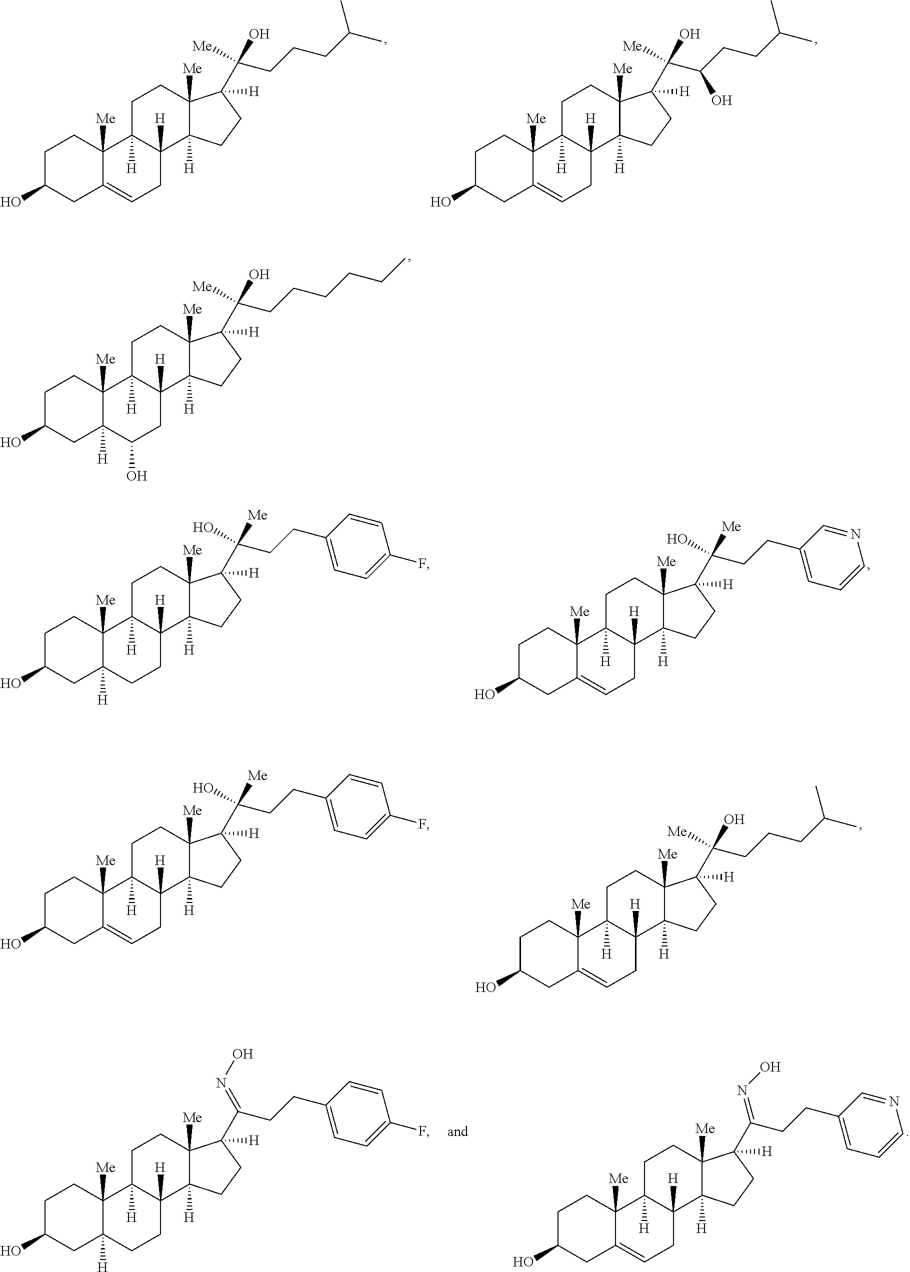

Additional exemplary compounds of Formula (I) are described, for example, in U.S. Pat. No. 9,637,514, US Patent Publication No. 20170189429, US Patent Publication No. 20180311259, and U.S. Provisional Application No. 63/035,597, filed Jun. 5, 2020, contents of all of which are incorporated herein by reference in their entireties.

### Synthesis of the Compounds

The synthesis of compounds described herein can be accomplished using means described in the chemical literature. For example, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999)(all of which are incorporated by reference for such disclosure). General methods for the preparation of compound as disclosed herein may be derived from reactions and the reactions may be modified by the use of appropriate reagents and conditions, for the introduction of the various moieties found in the formulae as provided herein. For example, compounds described herein can be synthesized using methods described, for example, in U.S. Pat. No. 9,637,514, US Patent Publication No. 20170189429, US Patent Publication No. 20180311259, and U.S. Provisional Application No. 63/035,597, filed Jun. 5, 2020.

### Routes of Administration

It is noted that the terms “administered” and “subjected” are used interchangeably in the context of treatment of a disease or disorder. In jurisdictions that forbid the patenting of methods that are practiced on the human body, the meaning of “administering” of a composition to a human subject shall be restricted to prescribing a controlled substance that a human subject will be administer to the subject by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrastemal injection and infusion. In some embodiments, administration will generally be local rather than systemic.

In some embodiments, a compound of Fomrula (I) is orally administered. Without limitations, oral administration can be in the form of solutions, suspensions, tablets, pills, capsules, sustained-release formulations, oral rinses, powders and the like.

In some embodiments, a compound of Formula (I) is compound is administered in a local rather than systemic manner, for example, via topical application of the compound directly on to skin, or intravenously, or subcutaneously, often in a depot preparation or sustained release formulation. In specific embodiments, long acting formulations are administered by implantation (for example subcutaneously or intramuscularly) or by intramuscular injection. In yet other embodiments, the compound as described herein is provided in the form of a rapid release formulation, in the form of an extended release formulation, or in the form of an intermediate release formulation. In yet other embodiments, the compound described herein is administered topically (e.g., as a patch, an ointment, or in combination with a wound dressing, or as a wash or a spray). In alternative embodiments, a formulation is administered systemically (e.g., by injection, or as a pill).

The phrase “therapeutically-effective amount” as used herein means that amount of a compound, material, or composition comprising a compound described herein which is effective for producing some desired therapeutic effect in at least a sub-population of cells, e.g., modulate or inhibit activity of MAGL in a subject at a reasonable benefit/risk ratio applicable to any medical treatment. Thus, “therapeutically effective amount” means that amount which, when administered to a subject for treating a disease, is sufficient to affect such treatment for the disease.

Depending on the route of administration, effective doses can be calculated according to the body weight, body surface area, or organ size of the subject to be treated. Optimization of the appropriate dosages can readily be made by one skilled in the art in light of pharmacokinetic data observed in human clinical trials. Alternatively, or additionally, the dosage to be administered can be determined from studies using animal models for the particular type of condition to be treated, and/or from animal or human data obtained from agents which are known to exhibit similar pharmacological activities. The final dosage regimen will be determined by the attending surgeon or physician, considering various factors which modify the action of active agent, e.g., the agent's specific activity, the agent's specific half-life in vivo, the severity of the condition and the responsiveness of the patient, the age, condition, body weight, sex and diet of the patient, the severity of any present infection, time of administration, the use (or not) of other concomitant therapies, and other clinical factors.

Determination of an effective amount is well within the capability of those skilled in the art. Generally, the actual effective amount can vary with the specific compound, the use or application technique, the desired effect, the duration of the effect and side effects, the subject's history, age, condition, sex, as well as the severity and type of the medical condition in the subject, and administration of other pharmaceutically active agents. Accordingly, an effective dose of compound described herein is an amount sufficient to produce at least some desired therapeutic effect in a subject.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of use or administration utilized.

The effective dose can be estimated initially from cell culture assays. A dose can be formulated in animal models to achieve a circulating plasma concentration range that includes the IC₅₀ (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay. The effective plasma concentration for a compound as disclosed herein can be about 0.01 μM to about 10 μM, about 0.2 μM to about 5 μM, or about 0.8 to about 3 μM in a subject, such as a rat, dog, or human.

Generally, the compositions are administered so that a compound of the disclosure herein is used or given at a dose from 1 μg/kg to 1000 mg/kg; 1 μg/kg to 500 mg/kg; 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. Further contemplated is a dose (either as a bolus or continuous infusion) of about 0.1 mg/kg to about 10 mg/kg, about 0.3 mg/kg to about 5 mg/kg, or 0.5 mg/kg to about 3 mg/kg. It is to be further understood that the ranges intermediate to those given above are also within the scope of this disclosure, for example, in the range 1 mg/kg to 10 mg/kg, for example use or dose ranges such as 2 mg/kg to 8 mg/kg, 3 mg/kg to 7 mg/kg, 4 mg/kg to 6 mg/kg, and the like.

The compounds described herein can be administered at once, or can be divided into a number of smaller doses to be administered at intervals of time. It is understood that the precise dosage and duration of treatment will be a function of the location of where the composition is parenterally administered, the carrier and other variables that can be determined empirically using known testing protocols or by extrapolation from in vivo or in vitro test data. It is to be noted that concentrations and dosage values can also vary with the age of the individual treated. It is to be further understood that for any particular subject, specific dosage regimens can need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The compound can be administered as a single bolus or multiple boluses, as a continuous infusion, or a combination thereof. For example, the compound can be administered as a single bolus initially, and then administered as a continuous infusion following the bolus. The rate of the infusion can be any rate sufficient to maintain effective concentration, for example, to maintain effective plasma concentration. Some contemplated infusion rates include from 1 μg/kg/min to 100 mg/kg/min, or from 1 μg/kg/hr to 1000 mg/kg/hr. Rates of infusion can include 0.2 to 1.5 mg/kg/min, or more specifically 0.25 to 1 mg/kg/min, or even more specifically 0.25 to 0.5 mg/kg/min. It will be appreciated that the rate of infusion can be determined based upon the dose necessary to maintain effective plasma concentration and the rate of elimination of the compound, such that the compound is administered via infusion at a rate sufficient to safely maintain a sufficient effective plasma concentration of compound in the bloodstream.

### Pharmaceutical Compositions/Formulations

For administration to a subject, the compounds of Formula (I) can be provided in pharmaceutically acceptable compositions. These pharmaceutically acceptable compositions comprise a compound of Formula (I), formulated together with one or more pharmaceutically acceptable carriers (additives) and/or diluents. As described in detail below, the pharmaceutical compositions described herein can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Examples of solid carriers include starch, sugar, bentonite, silica, and other commonly used carriers. Further non-limiting examples of carriers and diluents which can be used in the formulations comprising a compound of Formula (I) as disclosed herein of the present invention include saline, syrup, dextrose, and water.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

The phrase “therapeutically-effective amount” as used herein means that amount of a compound, material, or composition which is effective for producing some desired therapeutic effect in at least a sub-population of cells in an animal at a reasonable benefit/risk ratio applicable to any medical treatment. According, a “therapeutically effective amount” refers to an amount effective, at dosage and periods of time necessary, to achieve a desired therapeutic result. A therapeutic result can be, e.g., lessening of symptoms, prolonged survival, improved mobility, and the like. A therapeutic result need not be a “cure.”

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The compounds can be formulated in a gelatin capsule, in tablet form, dragee, syrup, suspension, topical cream, suppository, injectable solution, or kits for the preparation of syrups, suspension, topical cream, suppository or injectable solution just prior to use. Also, compounds can be included in composites, which facilitate its slow release into the blood stream, e.g., silicon disc, polymer beads.

The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171. Methods to make invention formulations include the step of bringing into association or contacting an ActRIIB compound with one or more excipients or carriers. In general, the formulations are prepared by uniformly and intimately bringing into association one or more compounds with liquid excipients or finely divided solid excipients or both, and then, if appropriate, shaping the product.

The preparative procedure may include the sterilization of the pharmaceutical preparations. The compounds may be mixed with auxiliary agents such as lubricants, preservatives, stabilizers, salts for influencing osmotic pressure, etc., which do not react deleteriously with the compounds.

Examples of injectable form include solutions, suspensions and emulsions. Injectable forms also include sterile powders for extemporaneous preparation of injectable solutions, suspensions or emulsions. The compounds of the present invention can be injected in association with a pharmaceutical carrier such as normal saline, physiological saline, bacteriostatic water, Cremophor™ EL (BASF, Parsippany, N.J.), phosphate buffered saline (PBS), Ringer's solution, dextrose solution, ethanol, polyol (e.g., glycerol, propylene glycol, and liquid polyethylene glycol), vegetable oils, and suitable mixtures thereof, and other aqueous carriers known in the art. Appropriate non-aqueous carriers may also be used and examples include fixed oils and ethyl oleate. In all cases, the composition must be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin. A suitable carrier is 5% dextrose in saline. Frequently, it is desirable to include additives in the carrier such as buffers and preservatives or other substances to enhance isotonicity and chemical stability.

In some embodiments, compounds can be administrated encapsulated within liposomes. The manufacture of such liposomes and insertion of molecules into such liposomes being well known in the art, for example, as described in U.S. Pat. No. 4,522,811. Liposomal suspensions (including liposomes targeted to particular cells, e.g., a pituitary cell) can also be used as pharmaceutically acceptable carriers.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Chemg-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185; content of each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments, the compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In the case of oral ingestion, excipients useful for solid preparations for oral administration are those generally used in the art, and the useful examples are excipients such as lactose, sucrose, sodium chloride, starches, calcium carbonate, kaolin, crystalline cellulose, methyl cellulose, glycerin, sodium alginate, gum arabic and the like, binders such as polyvinyl alcohol, polyvinyl ether, polyvinyl pyrrolidone, ethyl cellulose, gum arabic, shellac, sucrose, water, ethanol, propanol, carboxymethyl cellulose, potassium phosphate and the like, lubricants such as magnesium stearate, talc and the like, and further include additives such as usual known coloring agents, disintegrators such as alginic acid and Primogel™, and the like. The compounds can be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these compounds may be incorporated with excipients and used in the form of tablets, capsules, elixirs, suspensions, syrups, and the like. Such compositions and preparations should contain at least 0.1% of compound. The percentage of the agent in these compositions may, of course, be varied and may conveniently be between about 2% to about 60% of the weight of the unit. The amount of compound in such therapeutically useful compositions is such that a suitable dosage will be obtained. Preferred compositions according to the present invention are prepared so that an oral dosage unit contains between about 100 and 2000 mg of compound. Examples of bases useful for formulation of suppositories are oleaginous bases such as cacao butter, polyethylene glycol, lanolin, fatty acid triglycerides, witepsol (trademark, Dynamite Nobel Co. Ltd.) and the like. Liquid preparations may be in the form of aqueous or oleaginous suspension, solution, syrup, elixir and the like, which can be prepared by a conventional way using additives. The compositions can be given as a bolus dose, to maximize the circulating levels for the greatest length of time after the dose. Continuous infusion may also be used after the bolus dose.

The compounds can also be administrated directly to the airways in the form of an aerosol. For administration by inhalation, the compounds in solution or suspension can be delivered in the form of an aerosol spray from pressured container or dispenser which contains a suitable propellant, e.g., a gas such as carbon dioxide, or hydrocarbon propellant like propane, butane or isobutene. The compounds can also be administrated in a no-pressurized form such as in an atomizer or nebulizer.

In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

Representative intranasal formulations are described in, for example, U.S. Pat. Nos. 4,476,116, 5,116,817 and 6,391,452. Formulations that include a compound of Formula (I) are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions

The compounds can also be administered parenterally. Solutions or suspensions of these compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

It may be advantageous to formulate oral or parenteral compositions in dosage unit form for ease of administration and uniformity of dosage. As used herein, “dosage unit” refers to physically discrete units suited as unitary dosages for the subject to be treated; each unit containing a predetermined quantity of compound calculated to produce the desired therapeutic effect in association with the required pharmaceutical carrier.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

For oral or enteral formulations as disclosed herein for use with the present invention, tablets can be formulated in accordance with conventional procedures employing solid carriers well-known in the art. Capsules employed for oral formulations to be used with the methods of the present invention can be made from any pharmaceutically acceptable material, such as gelatin or cellulose derivatives. Sustained release oral delivery systems and/or enteric coatings for orally administered dosage forms are also contemplated, such as those described in U.S. Pat. No. 4,704,295, “Enteric Film-Coating Compositions,” issued Nov. 3, 1987; U.S. Pat. No. 4,556,552, “Enteric Film-Coating Compositions,” issued Dec. 3, 1985; U.S. Pat. No. 4,309,404, “Sustained Release Pharmaceutical Compositions,” issued Jan. 5, 1982; and U.S. Pat. No. 4,309,406, “Sustained Release Pharmaceutical Compositions,” issued Jan. 5, 1982. As regards formulations for administering a compound of Formula I as disclosed herein, one particularly useful embodiment

Also provided herein is a tablet formulation comprising a compound of Formula I with an enteric polymer casing. An example of such a preparation can be found in WO2005/021002. The active material in the core can be present in a micronised or solubilised form. In addition to active materials the core can contain additives conventional to the art of compressed tablets. Appropriate additives in such a tablet can comprise diluents such as anhydrous lactose, lactose monohydrate, calcium carbonate, magnesium carbonate, dicalcium phosphate or mixtures thereof; binders such as microcrystalline cellulose, hydroxypropylmethylcellulose, hydroxypropyl-cellulose, polyvinylpyrrolidone, pre-gelatinised starch or gum acacia or mixtures thereof; disintegrants such as microcrystalline cellulose (fulfilling both binder and disintegrant functions) cross-linked polyvinylpyrrolidone, sodium starch glycollate, croscarmellose sodium or mixtures thereof; lubricants, such as magnesium stearate or stearic acid, glidants or flow aids, such as colloidal silica, talc or starch, and stabilisers such as desiccating amorphous silica, colouring agents, flavours etc. Preferably the tablet comprises lactose as diluent. When a binder is present, it is preferably hydroxypropylmethyl cellulose. Preferably, the tablet comprises magnesium stearate as lubricant. Preferably the tablet comprises croscarmellose sodium as disintegrant. Preferably, the tablet comprises microcrystalline cellulose.

The diluent can be present in a range of 10-80% by weight of the core. The lubricant can be present in a range of 0.25-2% by weight of the core. The disintegrant can be present in a range of 1-10% by weight of the core. Microcrystalline cellulose, if present, can be present in a range of 10-80% by weight of the core.

The active ingredient, e.g., compound of Formula I preferably comprises between 10 and 50% of the weight of the core, more preferably between 15 and 35% of the weight of the core (calculated as free base equivalent). The core can contain any therapeutically suitable dosage level of the active ingredient, but preferably contains up to 150 mg of the active ingredient. Particularly preferably, the core contains 20, 30, 40, 50, 60, 80 or 100 mg of the active ingredient. The active ingredient can be present as is or as any pharmaceutically acceptable salt. If the active ingredient is present as a salt, the weight is adjusted such that the tablet contains the desired amount of active ingredient, calculated as free base or free acid of the salt.

The core can be made from a compacted mixture of its components. The components can be directly compressed, or can be granulated before compression. Such granules can be formed by a conventional granulating process as known in the art. In an alternative embodiment, the granules can be individually coated with an enteric casing, and then enclosed in a standard capsule casing.

The core is surrounded by a casing which comprises an enteric polymer. Examples of enteric polymers are cellulose acetate phthalate, cellulose acetate succinate, methylcellulose phthalate, ethylhydroxycellulose phthalate, polyvinylacetate pthalate, polyvinylbutyrate acetate, vinyl acetate-maleic anhydride copolymer, styrene-maleic mono-ester copolymer, methyl acrylate-methacrylic acid copolymer or methacrylate-methacrylic acid-octyl acrylate copolymer. These can be used either alone or in combination, or together with other polymers than those mentioned above. The casing can also include insoluble substances which are neither decomposed nor solubilised in living bodies, such as alkyl cellulose derivatives such as ethyl cellulose, crosslinked polymers such as styrene-divinylbenzene copolymer, polysaccharides having hydroxyl groups such as dextran, cellulose derivatives which are treated with bifunctional crosslinking agents such as epichlorohydrin, dichlorohydrin or 1, 2-, 3, 4-diepoxybutane. The casing can also include starch and/or dextrin.

In some embodiments, an entericcoating materials are the commercially available Eudragit® enteric polymers such as Eudragit® L, Eudragit® S and Eudragit® NE used alone or with a plasticiser. Such coatings are normally applied using a liquid medium, and the nature of the plasticiser depends upon whether the medium is aqueous or non-aqueous. Plasticisers for use with aqueous medium include propylene glycol, triethyl citrate, acetyl triethyl citrate or Citroflex® or Citroflex® A2. Non-aqueous plasticisers include these, and also diethyl and dibutyl phthalate and dibutyl sebacate. A preferred plasticiser is Triethyl citrate. The quantity of plasticiser included will be apparent to those skilled in the art.

The casing can also include an anti-tack agent such as talc, silica or glyceryl monostearate. Preferably the anti-tack agent is glyceryl monostearate. Typically, the casing can include around 5-25 wt % Plasticizers and up to around 50 wt % of anti-tack agent, preferably 1-10 wt % of anti-tack agent.

If desired, a surfactant can be included to aid with forming an aqueous suspension of the polymer. Many examples of possible surfactants are known to the person skilled in the art. Preferred examples of surfactants are polysorbate 80, polysorbate 20, or sodium lauryl sulphate. If present, a surfactant can form 0.1-10% of the casing, preferably 0.2-5% and particularly preferably 0.5-2%.

A seal coat can also be included between the core and the enteric coating. A seal coat is a coating material which can be used to protect the enteric casing from possible chemical attack by any alkaline ingredients in the core. The seal coat can also provide a smoother surface, thereby allowing easier attachment of the enteric casing. A person skilled in the art would be aware of suitable coatings. Preferably the seal coat is made of an Opadry coating, and particularly preferably it is Opadry White OY-S-28876. Other enteric-coated preparations of this sort can be prepared by one skilled in the art, using these materials or their equivalents.

For intravenous injections or drips or infusions, compounds described herein are formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspension, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

### Definitions

For convenience, certain terms employed herein, in the specification, examples and appended claims are collected herein. Unless stated otherwise, or implicit from context, the following terms and phrases include the meanings provided below Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%, ±1.5%, ±2%, ±2.5%, ±3%, ±3.5%, +4%, +4.5%, or 5%.

The singular terms “a,” “an,” and “the” include plural referents unless context clearly indicates otherwise. Similarly, the word “or” is intended to include “and” unless the context clearly indicates otherwise.

As used herein the terms “comprising” or “comprises” means “including” or “includes” and are used in reference to compositions, methods, systems, and respective component(s) thereof, that are useful to the invention, yet open to the inclusion of unspecified elements, whether useful or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, systems, and respective components thereof as described herein, which are exclusive of any element not recited in that description of the embodiment.

The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

As used herein, the terms “treat,” “treatment,” “treating,” or “amelioration” are used herein to characterize a method or process that is aimed at (1) delaying or preventing the onset of a disease or condition; (2) slowing down or stopping the progression, aggravation, or deterioration of the symptoms of the disease or condition; (3) bringing about ameliorations of the symptoms of the disease or condition; or (4) curing the disease or condition. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased morbidity or mortality. The term “treatment” of a disease also includes providing relief from the symptoms or side-effects of the disease (including palliative treatment). A treatment can be administered prior to the onset of the disease, for a prophylactic or preventive action. Alternatively, or additionally, the treatment can be administered after initiation of the disease or condition, for a therapeutic action.

In some embodiments, treatment is therapeutic and does not include prophylactic treatment.

The terms “co-administration” or the like, as used herein, are meant to encompass administration of the selected therapeutic agents to a single patient and are intended to include treatment regimens in which the agents are administered by the same or different route of administration or at the same or different time. The particular combination of therapies (therapeutics or procedures) to employ in such a combination regimen will take into account compatibility of the desired therapeutics and/or procedures and the desired therapeutic effect to be achieved.

As used herein, the term “subject” refers to any living organism which can be administered compound and/or pharmaceutical compositions of the present invention. The term includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term does not denote a particular age or sex. Thus, adult, child and newborn subjects, whether male or female, are intended to be covered. The term “subject” is also intended to include living organisms susceptible to conditions or disease states as generally disclosed, but not limited to, throughout this specification. Examples of subjects include humans, dogs, cats, cows, goats, and mice. The term subject is further intended to include transgenic species. The term “subject” and “individual” are used interchangeably herein, and refer to an animal, for example a human or non-human mammals/animals, to whom treatment, including prophylactic treatment, with the compounds and compositions according to the present invention, is provided. The term “non-human animals” and “non-human mammals” are used interchangeably herein and include all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc.

In some embodiments, the subject is a human or animal. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of viral-infections.

It is noted that a human subject can be of any age, gender, race or ethnic group, e.g., Caucasian (white), Asian, African, black, African American, African European, Hispanic, Middle eastern, etc. . . .

In addition, the methods described herein can be used to treat domesticated animals and/or pets. A subject can be male or female. A subject can be one who has been previously diagnosed with or identified as suffering from or having a viral infection, but need not have already undergone treatment.

In some embodiments of any one of the aspects, the subject is human.

As used herein, the term “alkyl” refers to an aliphatic hydrocarbon group which can be straight or branched having 1 to about 60 carbon atoms in the chain, and which preferably have about 6 to about 50 carbons in the chain. “Lower alkyl” refers to an alkyl group having 1 to about 8 carbon atoms. “Higher alkyl” refers to an alkyl group having about 10 to about 20 carbon atoms. The alkyl group can be optionally substituted with one or more alkyl group substituents which can be the same or different, where “alkyl group substituent” includes halo, amino, aryl, hydroxy, alkoxy, aryloxy, alkyloxy, alkylthio, arylthio, aralkyloxy, aralkylthio, carboxy, alkoxycarbonyl, oxo and cycloalkyl. “Branched” refers to an alkyl group in which a lower alkyl group, such as methyl, ethyl or propyl, is attached to a linear alkyl chain. Exemplary alkyl groups include methyl, ethyl, propyl, i-propyl, n-butyl, t-butyl, n-pentyl, hexyl, heptyl, octyl, decyl, dodecyl, tridecyl, tetradecyl, pentadecyl and hexadecyl. Useful alkyl groups include branched or straight chain alkyl groups of 6 to 50 carbon, and also include the lower alkyl groups of 1 to about 4 carbons and the higher alkyl groups of about 12 to about 16 carbons.

A “heteroalkyl” group substitutes any one of the carbons of the alkyl group with a heteroatom having the appropriate number of hydrogen atoms attached (e.g., a CH₂ group to an NH group or an O group). The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃

As used herein, the term “alkenyl” refers to an alkyl group containing at least one carbon-carbon double bond. The alkenyl group can be optionally substituted with one or more “alkyl group substituents.” Exemplary alkenyl groups include vinyl, allyl, n-pentenyl, decenyl, dodecenyl, tetradecadienyl, heptadec-8-en-1-yl and heptadec-8,11-dien-1-yl.

As used herein, the term “alkynyl” refers to an alkyl group containing a carbon-carbon triple bond. The alkynyl group can be optionally substituted with one or more “alkyl group substituents.” Exemplary alkynyl groups include ethynyl, propargyl, n-pentynyl, decynyl and dodecynyl. Useful alkynyl groups include the lower alkynyl groups.

As used herein, the term “cycloalkyl” refers to a non-aromatic mono- or multicyclic ring system of about 3 to about 12 carbon atoms. The cycloalkyl group can be optionally partially unsaturated. The cycloalkyl group can be also optionally substituted with an aryl group substituent, oxo and/or alkylene. Representative monocyclic cycloalkyl rings include cyclopentyl, cyclohexyl and cycloheptyl. Useful multicyclic cycloalkyl rings include adamantyl, octahydronaphthyl, decalin, camphor, camphane, and noradamantyl.

“Heterocyclyl” refers to a nonaromatic 3-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

“Aryl” refers to an aromatic carbocyclic radical containing about 3 to about 13 carbon atoms. The aryl group can be optionally substituted with one or more aryl group substituents, which can be the same or different, where “aryl group substituent” includes alkyl, alkenyl, alkynyl, aryl, aralkyl, hydroxy, alkoxy, aryloxy, aralkoxy, carboxy, aroyl, halo, nitro, trihalomethyl, cyano, alkoxycarbonyl, aryloxycarbonyl, aralkoxycarbonyl, acyloxy, acylamino, aroylamino, carbamoyl, alkylcarbamoyl, dialkylcarbamoyl, rylthio, alkylthio, alkylene and —NRR′, where R and R′ are each independently hydrogen, alkyl, aryl and aralkyl. Exemplary aryl groups include substituted or unsubstituted phenyl and substituted or unsubstituted naphthyl.

“Heteroaryl” refers to an aromatic 3-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively.

Exemplary aryl and heteroaryls include, but are not limited to, phenyl, pyridinyl, pyrimidinyl, furanyl, thienyl, imidazolyl, thiazolyl, pyrazolyl, pyridazinyl, pyrazinyl, triazinyl, tetrazolyl, indolyl, benzyl, naphthyl, anthracenyl, azulenyl, fluorenyl, indanyl, indenyl, naphthyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application.

The term “haloalkyl” as used herein refers to alkyl and alkoxy structures structure with at least one substituent of fluorine, chorine, bromine or iodine, or with combinations thereof. In embodiments, where more than one halogen is included in the group, the halogens are the same or they are different. The terms “fluoroalkyl” and “fluoroalkoxy” include haloalkyl and haloalkoxy groups, respectively, in which the halo is fluorine. Exemplary halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (CF₃), perfluoroethyl, 2,2,2-trifluoroethyl, 2,2,2-trifluoro-1,1-dichloroethyl, and the like).

As used herein, the term “amino” means —NH₂. The term “alkylamino” means a nitrogen moiety having one straight or branched unsaturated aliphatic, cyclyl, or heterocyclyl radicals attached to the nitrogen, e.g., —NH(alkyl). The term “dialkylamino” means a nitrogen moiety having at two straight or branched unsaturated aliphatic, cyclyl, or heterocyclyl radicals attached to the nitrogen, e.g., —N(alkyl)(alkyl). The term “alkylamino” includes “alkenylamino,” “alkynylamino,” “cyclylamino,” and “heterocyclylamino.” The term “arylamino” means a nitrogen moiety having at least one aryl radical attached to the nitrogen. For example, —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like. Exemplary alkylamino includes, but is not limited to, NH(C₁-C₁₀alkyl), such as —NHCH₃, —NHCH₂CH₃, —NHCH₂CH₂CH₃, and —NHCH(CH₃)₂. Exemplary dialkylamino includes, but is not limited to, —N(C₁-C₁₀alkyl)₂, such as N(CH₃)₂, —N(CH₂CH₃)₂, —N(CH₂CH₂CH₃)₂, and —N(CH(CH₃)₂)₂.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The terms “hydroxy” and “hydroxyl” mean the radical —OH.

The terms “alkoxyl” or “alkoxy” as used herein refers to an alkyl group, as defined above, having an oxygen radical attached thereto, and can be represented by one of —O-alkyl, —O— alkenyl, and —O-alkynyl. Aroxy can be represented by —O-aryl or O-heteroaryl, wherein aryl and heteroaryl are as defined herein. The alkoxy and aroxy groups can be substituted as described above for alkyl. Exemplary alkoxy groups include, but are not limited to O-methyl, O-ethyl, O-n-propyl, O-isopropyl, O-n-butyl, O-isobutyl, O-sec-butyl, O-tert-butyl, O-pentyl, O-hexyl, O-cyclopropyl, O-cyclobutyl, O-cyclopentyl, O-cyclohexyl and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. As used herein, a carboxy group includes —COOH, i.e., carboxyl group.

The term “ester” refers to a chemical moiety with formula —C(═O)OR, where R is selected from the group consisting of alkyl, cycloalkyl, aryl, heteroaryl and heterocycloalkyl.

The term “cyano” means the radical —CN.

The term “nitro” means the radical —NO₂.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The terms “alkylthio” and “thioalkoxy” refer to an alkoxy group, as defined above, where the oxygen atom is replaced with a sulfur. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

“Acyl” refers to an alkyl-CO— group, wherein alkyl is as previously described. Exemplary acyl groups comprise alkyl of 1 to about 30 carbon atoms. Exemplary acyl groups also include acetyl, propanoyl, 2-methylpropanoyl, butanoyl and palmitoyl.

“Aroyl” means an aryl-CO— group, wherein aryl is as previously described. Exemplary aroyl groups include benzoyl and 1- and 2-naphthoyl.

“Arylthio” refers to an aryl-S— group, wherein the aryl group is as previously described. Exemplary arylthio groups include phenylthio and naphthylthio.

“Aralkyl” refers to an aryl-alkyl- group, wherein aryl and alkyl are as previously described. Exemplary aralkyl groups include benzyl, phenylethyl and naphthylmethyl.

“Aralkyloxy” refers to an aralkyl-O— group, wherein the aralkyl group is as previously described. An exemplary aralkyloxy group is benzyloxy.

“Aralkylthio” refers to an aralkyl-S— group, wherein the aralkyl group is as previously described. An exemplary aralkylthio group is benzylthio.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group, wherein one of R and R′ is hydrogen and the other of R and R′ is alkyl as previously described.

“Dialkylcarbamoyl” refers to R′RN—CO— group, wherein each of R and R′ is independently alkyl as previously described.

“Acyloxy” refers to an acyl-O— group, wherein acyl is as previously described. “Acylamino” refers to an acyl-NH— group, wherein acyl is as previously described. “Aroylamino” refers to an aroyl-NH— group, wherein aroyl is as previously described.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

For example, any alkyl, alkenyl, cycloalkyl, heterocyclyl, heteroaryl or aryl is optionally substituted with 1, 2, 3, 4 or 5 groups selected from OH, CN, SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]2, C(O)NH₂, COOH, COOMe, acetyl, (C₁-C₅)alkyl, O(C₁-C₅)alkyl, O(C₁-C₅)haloalkyl, (C₂-C₅)alkenyl, (C₂-C₅)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; or wherein any alkyl, cycloalkyl or heterocyclyl is optionally substituted with oxo; “m” and “p” are independently 1, 2, 3, 4, 5 or 6.

In some embodiments, an optionally substituted group is substituted with 1 substituent. In some other embodiments, an optionally substituted group is substituted with 2 independently selected substituents, which can be same or different. In some other embodiments, an optionally substituted group is substituted with 3 independently selected substituents, which can be same, different or any combination of same and different. In still some other embodiments, an optionally substituted group is substituted with 4 independently selected substituents, which can be same, different or any combination of same and different. In yet some other embodiments, an optionally substituted group is substituted with 5 independently selected substituents, which can be same, different or any combination of same and different.

An “isocyanato” group refers to a NCO group.

A “thiocyanato” group refers to a CNS group.

An “isothiocyanato” group refers to a NCS group.

“Alkoyloxy” refers to a RC(═O)O— group.

“Alkoyl” refers to a RC(═O)— group.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

### EXAMPLES

### Example 1: Anti-HBV Activity of Exemplary Compounds of Formula (I)

### Materials and Methods

Cell culture: HepG2-hNTCP-C4 and Hep38.7-Tet cells were cultured with the media composed of DMEM/F-12+GlutaMax supplemented with 10 mM HEPES, 100 units/mL penicillin, 100 μg/mL streptomycin, 10% FBS, 5 μg/mL insulin, and 500 μg/mL G418.

HBV Preparation and Infection: HBV derived from the culture supernatant of Hep38.7-Tet cells (genotype D) was used as the HBV inoculum. HBV was inoculated at 12,000 genome equivalents (GEq) per cell in the presence of 4% polyethylene glycol 8000 at 37° C. for 16 h. After washing out to remove free virus, the cells were cultured for an additional 12 days.

Detection of HBs Antigens: HBs protein was quantified by ELISA using plates incubated at 4° C. overnight with a sheep anti-HBs antibody at 1:5000 dilution followed by coating with 0.2% BSA, 0.02% NaN₃, 1×PBS at 4° C. Samples were incubated with the plates for 2 h and after washing with TBST four times, horse-radish peroxidase-labeled rabbit anti-HBs antibody was added for 2 h. The substrate solution (S-BIO SUMILON) was reacted for 10 min to measure A₄₅₀ values with xMark microplate spectrophotometer (Bio-Rad).

Real-Time PCR: HBV DNA was extracted from cells using a QIAamp mini kit (QIAGEN) according to the manufacturer's protocol. HBV DNA was quantified by real time PCR using the primer set 5′-AAGGTAGGAGCTGGAGCATTCG-3′ (SEQ ID NO: 1) and 5′-AGGCGGATTTGCTGGCAAAG-3′ (SEQ ID NO: 2) and a probe 5′FAM-AGCCCTCAGGCTCAGGGCATAC-TAMRA3′ (SEQ ID NO: 3). cccDNA was detected using 5′-CGTCTGTGCCTTCTCATCTGC-3′ (SEQ ID NO: 4) and 5′-GCACAGCTTGGAGGCTTGAA-3′ (SEQ ID NO: 5) as primers and 5′FAM-CTGTAGGCATAAATTGGT-MGB-3′ (SEQ ID NO: 6) as a probe.

MTT Assay: Cell viability was measured by 3-(4, 5-dimethylthial-2-yl)-2, 5-diphenyltetrazolium bromide (MTT) assay using the Cell Proliferation Kit II (Sigma-Aldrich) according to the manufacturer's protocol.

Indirect Immunofluorescence Analysis: Cells were fixed with 4% paraformaldehyde and were permeabilized with 0.3% Triton X-100. The cells were treated with anti-HBc antibody (Thermo Fisher Scientific, Waltham, MA) at a dilution of 1:100 or anti-HDAg antibody at a dilution of 1:5,000 and then visualized with donkey anti-rabbit IgG (H+L) conjugated to Alexa 594 at a dilution of 1:500. The nucleus was stained with 4, 6-diamidino-2-phenylindole (DAPI) at a dilution of 1:5,000. Fluorescences were observed with a fluorescence microscope BZ-X700 (KEYENCE).

HBV replication assay: Hep38.7-Tet cells, a cell line that can induce HBV replication by depletion of tetracycline, were treated with compounds in the absence of tetracycline for 6 days and total HBV DNA intermediates in the cells were recovered and quantified by real-time PCR.

PreS1 binding assay: HBV preS1-mediated attachment to host cells was evaluated by exposing HepG2-hNTCP-C4 cells to 40 nM C-terminally TAMRA-conjugated and N-terminally myristoylated preS1 peptide, which spans amino acid 2-48 of the preS1 region of HBV (preS1-TAMRA), at 37° C. for 30 min. The cells then were washed, fixed with 4% paraformaldehyde, stained with DAPI, and observed for fluorescence by microscopy.

PreS1 internalization assay: HepG2-hNTCP-C4 cells were inoculated with 40 nM preS1-TAMRA at 4° C. for 30 min to allow attachment of the preS1 peptide to the cell surface. The cells were then transferred to 37° C. for 8 h to allow incorporation of the preS1-peptide into the cells. The cells then were washed, fixed with 4% paraformaldehyde, and treated with anti-NTCP antibody at a dilution of 1:20 and goat anti-mouse IgG (H+L) conjugated to Alexa 488 at a dilution of 1:1,000 to stain NTCP. The nucleus was stained with DAPI. These fluorescence signals were observed by using confocal microscopy TCS SP8 (Leica Microsystems).

### Results and Discussion

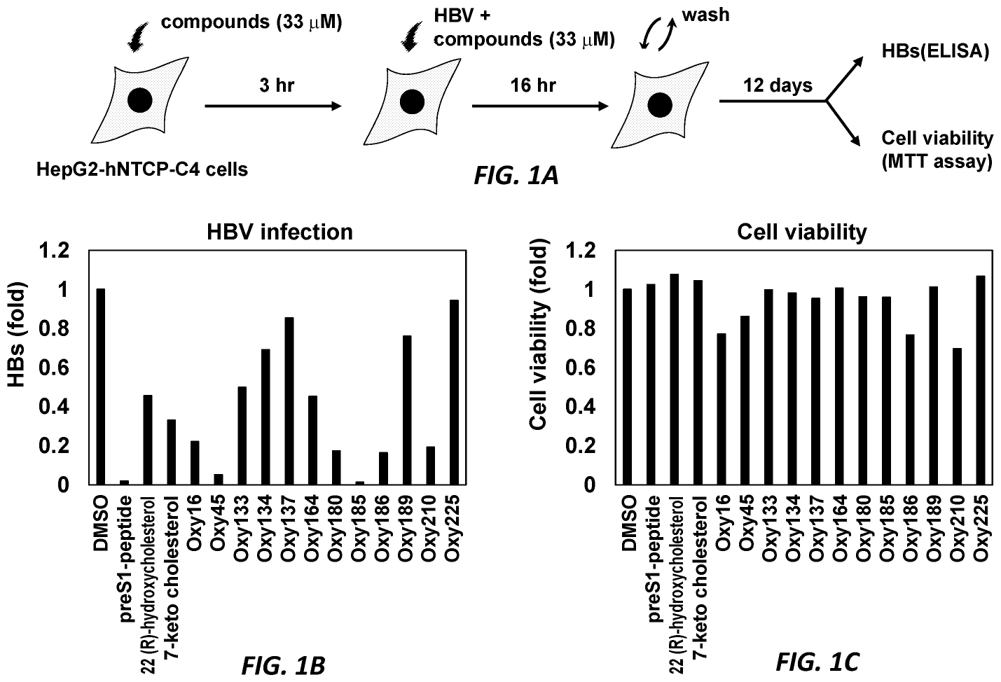

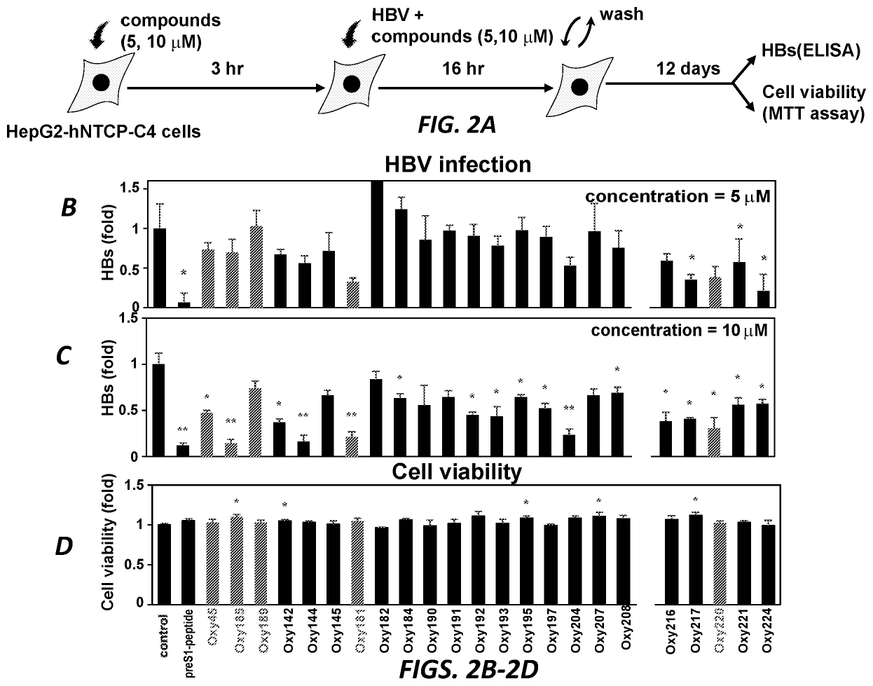

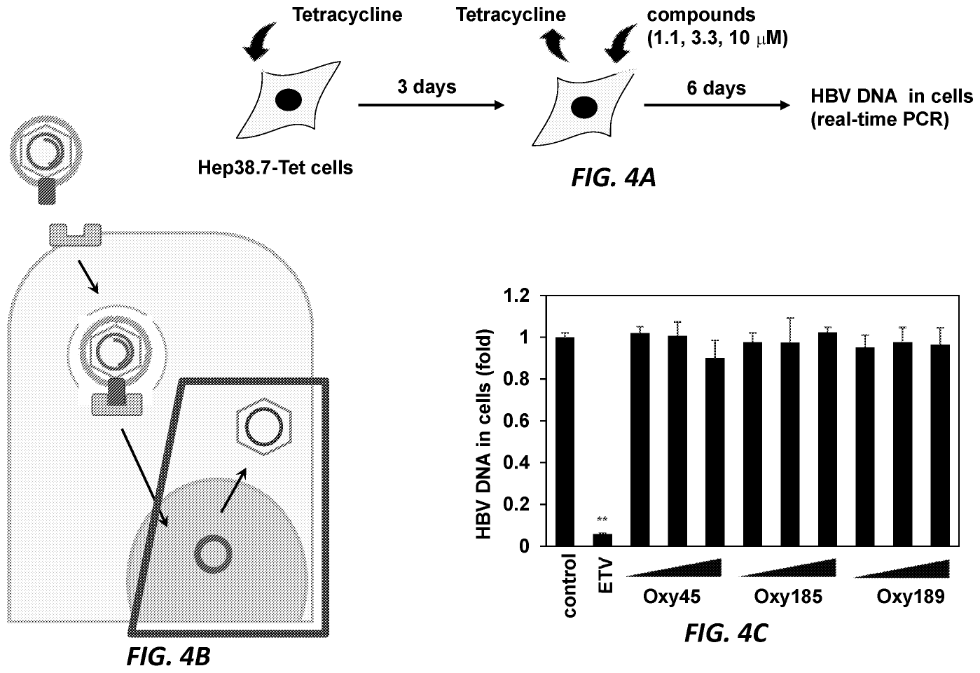

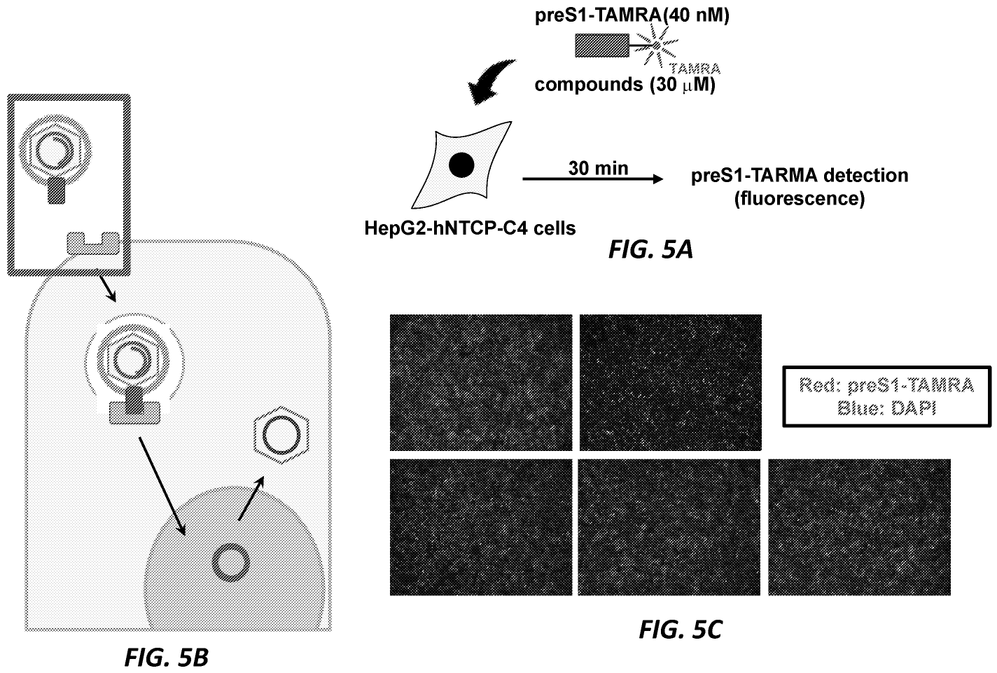

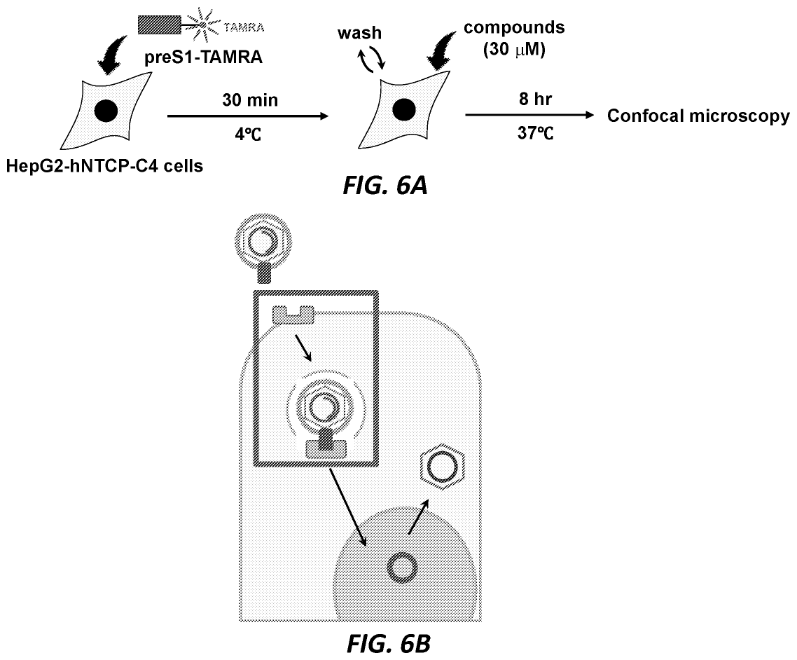

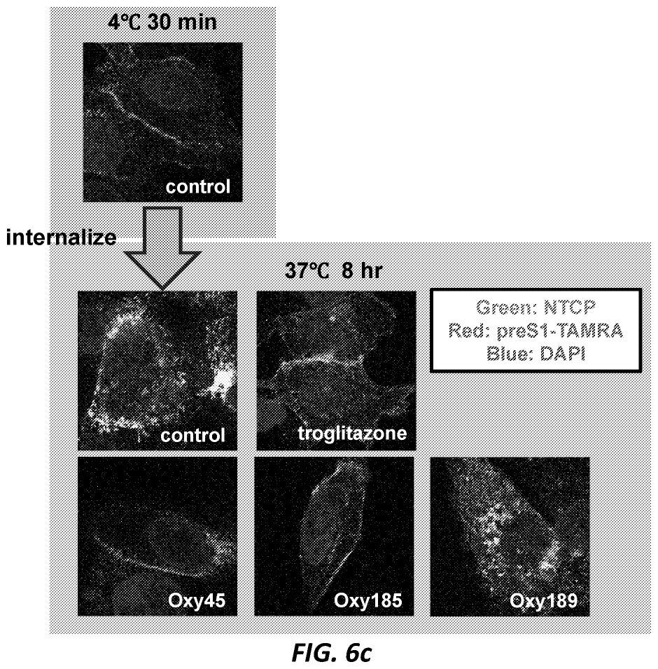

Results are shown in FIGS. 1B, 2B-2D, 3B, 4C, 5C and 6C.

### Example 2: Anti-HDV Activity of Exemplary Compounds of Formula (I)

### Materials and Methods

Hepatitis D Virus (HDV) Infection Assay: HDV was recovered from culture supernatants of Huh-7 cells transfected with pSVLD3 (kindly provided by Dr. John Taylor (Gudima et al. J Virol 2007)) and pT7HB2.7 (kindly provided by Dr. Camille Sureau (Kuo et al. J Virol 1989; Sureau et al. J Virol 1994)). HepG2-hNTCP-C4 cells were incubated with HDV at 25 GEq/cell in 5% PEG 8000 for 16 h, followed by washing out free virus and culturing of the cells for 6 additional days. HDAg produced by HDV replication was detected by immunofluorescence analysis.

### Results and Discussion

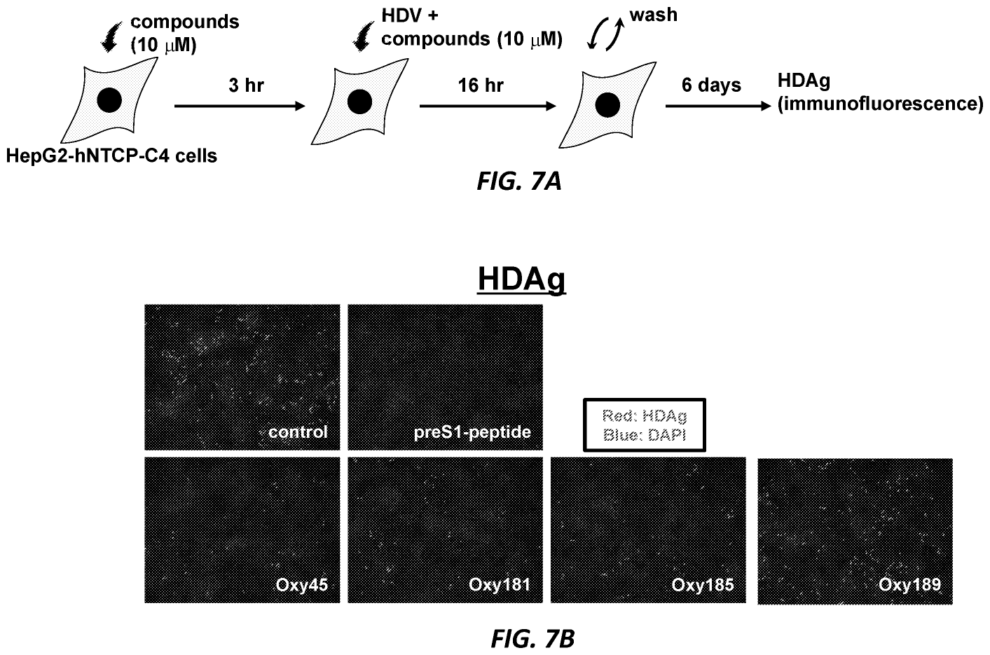

Results are shown in FIG. 7B.

### Example 3: Identification of Anti-Severe Acute Respiratory Syndrome-Related Coronavirus 2 (SARS-CoV-2) Oxysterol Derivatives In Vitro

The development of effective antiviral drugs targeting the severe acute respiratory syndrome-related coronavirus 2 (SARS-CoV-2) is urgently needed to combat the coronavirus disease 2019 (COVID-19). Inventors have previously studied the use of semi-synthetic derivatives of oxysterols, oxidized derivatives of cholesterol as drug candidates for the inhibition of cancer, fibrosis, and bone regeneration. In this study, inventors screened a panel of naturally occurring and semi-synthetic oxysterols for anti-SARS-CoV-2 activity using a cell culture infection assay. This study shows that the natural oxysterols, 7-ketocholesterol, 22(R)-hydroxycholesterol, 24(S)-hydroxycholesterol, and 27-hydroxycholesterol, substantially inhibited SARS-CoV-2 propagation in cultured cells. Among semi-synthetic oxysterols, Oxy210 and Oxy232 displayed more robust anti-SARS-CoV-2 activities, reducing viral replication more than 90% at 10 μM and 99% at 15 μM, respectively. When orally administered in mice, peak plasma concentrations of Oxy210 fell into a therapeutically relevant range (19 μM), based on the dose-dependent curve for antiviral activity in the cell-based assay described herein. Mechanistic studies indicate that Oxy210 reduced replication of SARS-CoV-2 by disrupting the formation of double-membrane vesicles (DMVs); intracellular membrane compartments associated with viral replication.

### Introduction

Coronavirus disease 2019 (COVID-19), caused by infection with the severe acute respiratory syndrome-related coronavirus 2 (SARS-CoV-2), has drastically impacted public health and, on a global scale, caused enormous harm to human societies and their economic vitality. In the search for effective treatments for COVID-19, understandably, the repurposing of existing FDA-approved drugs has been given high priority due to their known safety profiles [1]. For example, remdesivir (RDV), which was originally designed as an anti-ebola virus agent, has been repurposed to become the first and, to date, the only FDA-approved drug treatment for SARS-CoV-2 infection. Similarly, chloroquine (CLQ) and hydroxychloroquine, which are used to control malaria, have been investigated as COVID-19 treatments [2]. Beyond drug repurposing [3], other approaches are urgently needed to invigorate discovery research for new, specific, and potent anti-COVID-19 drugs.

Naturally occurring oxysterols include metabolites of cholesterol involved in the biosynthesis of steroid hormones, vitamin D, bile acids, and other crucial signaling molecules [4,5]. Beyond their role as passive and transient metabolites, endogenous oxysterols are increasingly recognized as lipid signaling molecules that can regulate a range of physiological processes, including lipid homeostasis, transport, and metabolism, as well as the immune response [5]. In recent years, numerous reports have ascribed broad-spectrum antiviral properties to naturally occurring oxysterols. For example, 20(S)-hydroxycholesterol (20(S)-OHC) and 22(S)-hydroxycholesterol (22(S)-OHC) reduced the infection of hepatitis B virus [6]; 25-hydroxycholesterol (25-OHC) and 27-hydroxycholesterol (27-OHC) displayed antiviral activities against herpes simplex virus [7], human papillomavirus-16, human rhinovirus [8], murine norovirus [9], rotavirus [10], and Zika virus [11].

In this study, the inventors focused on oxysterols, including naturally occurring and semi-synthetic oxysterols, to identify potent anti-SARS-CoV-2 agents since they have already developed various semi-synthetic oxysterols as drug candidates in the context of cancer, fibrotic diseases, and bone regeneration: Oxy133, an allosteric activator of Hedgehog (Hh) signaling, was designed for orthopedic applications, such as bone repair and spine fusion [12-14]; Oxy186, an inhibitor of Hh signaling that acts downstream of the Smoothened (Smo) receptor, was designed as a potential anti-tumorigenic agent [15], and Oxy210 was designed for application in cancer and fibrosis through dual inhibition of Hh and transforming growth factor β (TGFβ) signaling [16]. In the present report, using cell-based analysis, the inventors demonstrate that Oxy210 and its analog, Oxy232, display superior anti-SARS-CoV-2 activity compared to the natural oxysterols, 7-ketocholesterol (7-KC), 22(R)-hydroxycholesterol (22(R)-OHC), 24(S)-hydroxycholesterol (24(S)-OHC), and 27-OHC. Importantly, Oxy210 reduced viral replication and the formation of double-membrane vesicles (DMVs), known RNA replication factories of coronaviruses and other RNA viruses [17-19]. Oral administration of a single dose of Oxy210 at 200 mg/kg in mice resulted in a peak plasma concentration (Cₘₐₓ) of about 19 μM, which exceeds both the 50% maximal inhibitory concentration (IC₅₀) (5.5 μM) and 90% maximal inhibitory concentration (IC₉₀) (8.6 μM), respectively, determined by the cell-based assay described herein. These data provide foundational evidence for Oxy210 and Oxy232 as anti-COVID-19 therapeutics.

### Results

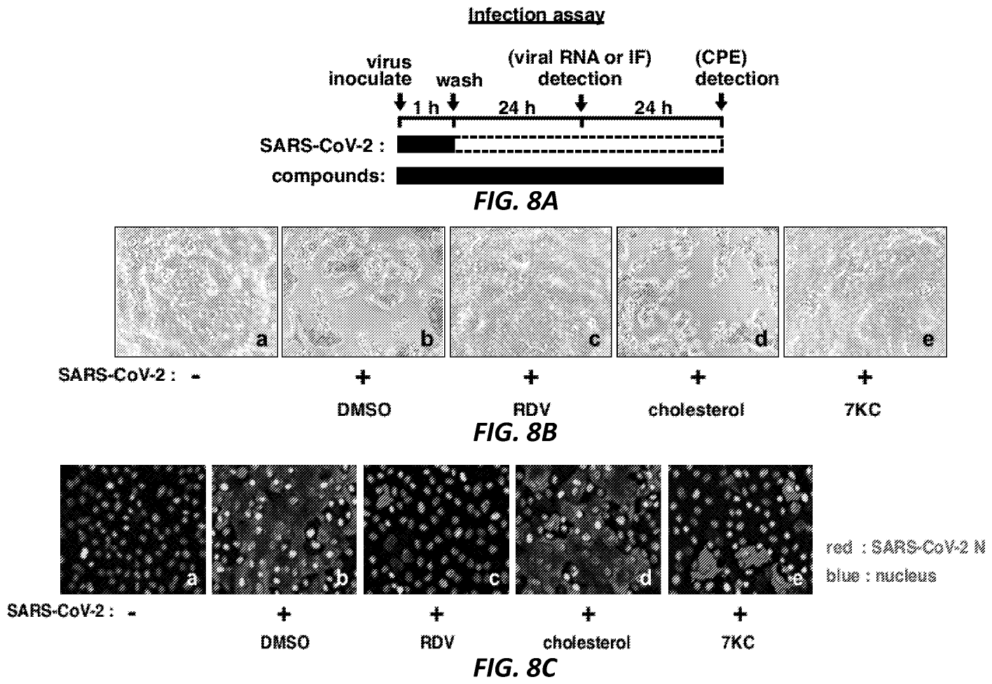

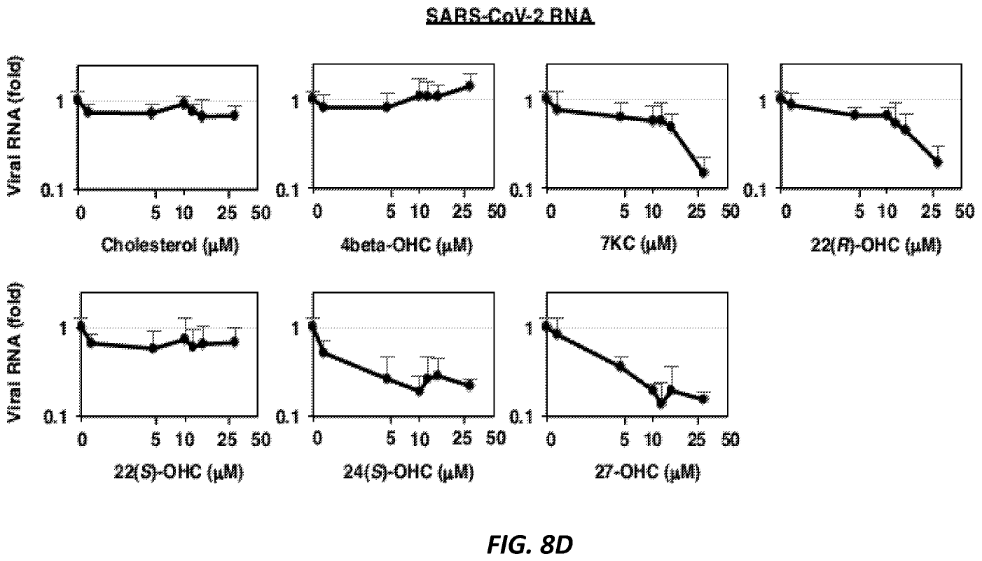

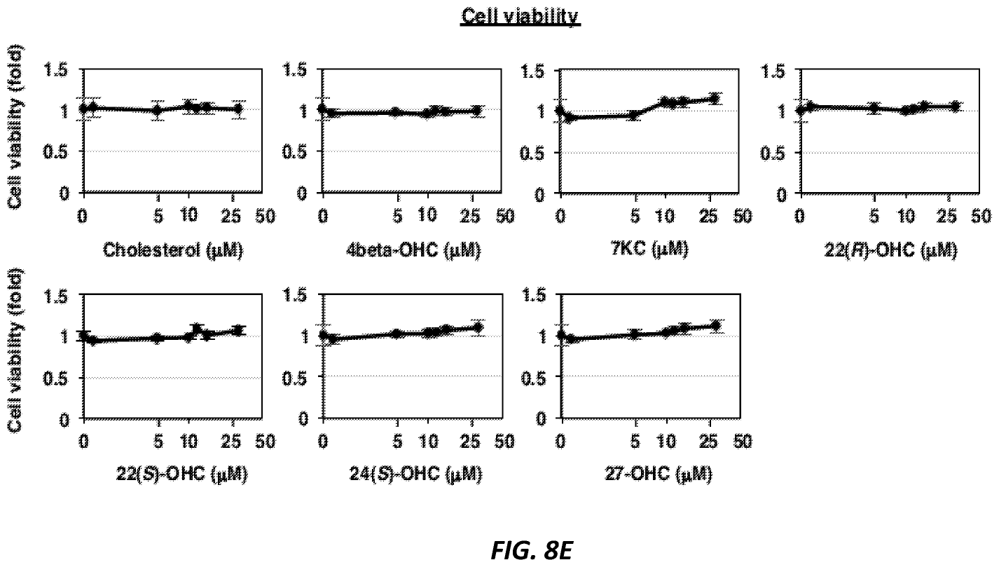

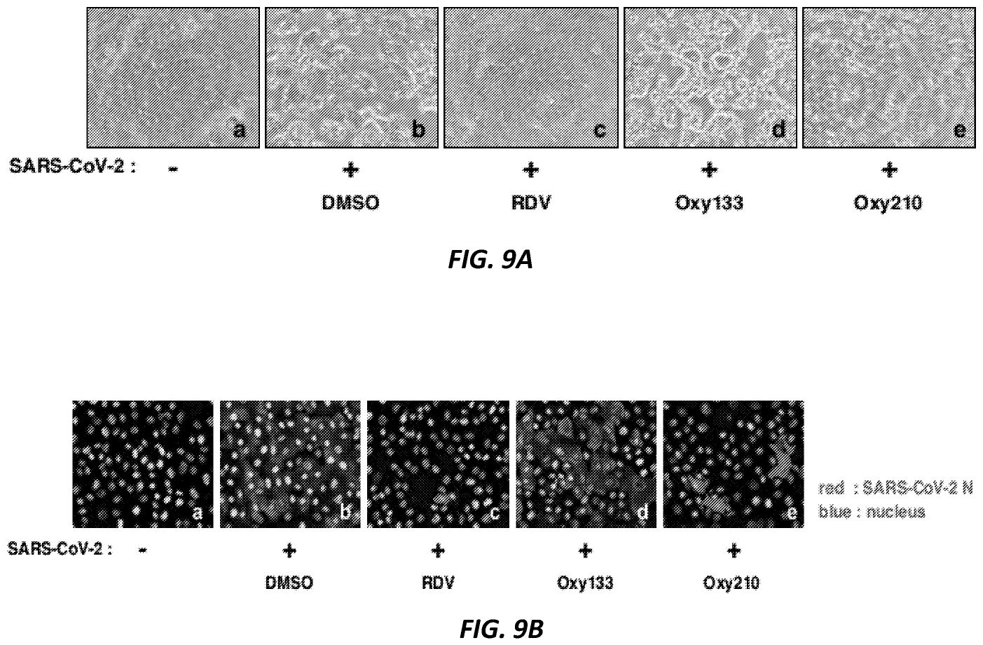

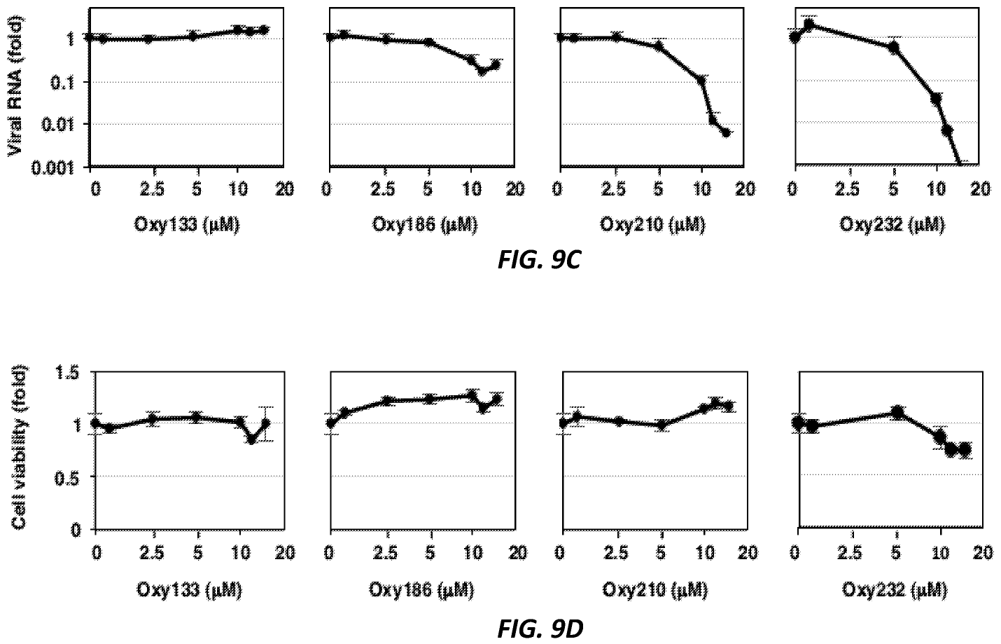

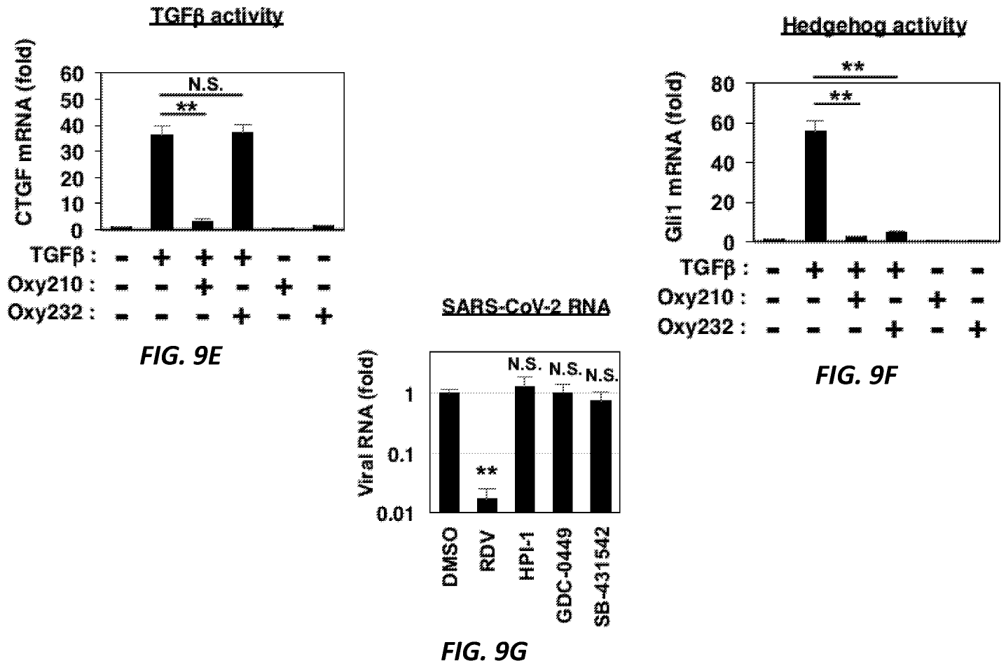

Natural Oxysterols have Antiviral Activity against SARS-CoV-2 Infection: In this study, inventors used a cell-based SARS-CoV-2 infection system previously reported [20]. This infection system uses VeroE6 cells stably overexpressing the TMPRSS2 gene, which is a member of type II transmembrane serine proteases. Cells were treated with test compounds for 1 h during inoculation with a clinical isolate of SARS-CoV-2 at a multiplicity of infection (MOI) of 0.001, followed by washing out free virus and incubating the cells with test compounds for 24 h or 48 h (FIG. 8A and Materials and Methods). SARS-CoV-2 propagation in VeroE6/TMPRSS2 cells induced a cytopathic effect (CPE) at 48 h post-virus inoculation (FIG. 8B, panel b), and the treatment with remdesivir (RDV), a known replication inhibitor of SARS-CoV-2 [2], blocked the virus-induced CPE (FIG. 8B, panel c). SARS-CoV-2 propagation visualized by detecting viral nucleocapsid (N) protein by immunofluorescence (IF) analysis was also blocked by RDV (FIG. 8C, panels b,c, red). Using this assay, we evaluated the antiviral effect of cholesterol and 7-ketocholesterol (7-KC) as a representative of natural oxysterols. 7-KC, but not cholesterol, reduced the SARS-CoV-2-induced CPE (FIG. 8B, panels d, e) and the spread of infection (FIG. 8C, panels d, e). To quantify antiviral activity, we measured viral RNA production in the culture supernatant and cell viability upon treatment with natural oxysterols or cholesterol at 24 h post-inoculation. Cholesterol, 4beta-hydroxysterol (4beta-OHC) and 22(S)-hydroxycholesterol (22(S)-OHC), did not show apparent reductions in viral RNA, while 7-KC, 22(R)-hydroxycholesterol (22(R)-OHC), 24(S)-hydroxycholesterol (24(S)-OHC), and 27-hydroxysterol (27-OHC) reduced the production of viral RNA by 80%-86% as compared to control (FIG. 8D). Evaluation of host cell viability showed no cytotoxic effect of the test compounds up to 30 μM, which is the maximum concentration in the SARS-CoV-2 infection assay shown in FIG. 9D and FIG. 9E. The IC₅₀s, IC₉₀s, and 50% maximal cytotoxic concentrations (CC₅₀s) for these compounds are summarized in Table 1. These findings show that the oxysterols inhibited SARS-CoV-2 propagation without showing cytotoxicity.

TABLE 1
The antiviral activities and cytotoxicities for all compounds.

| Compounds | IC₅₀ (μM) | IC₉₀ (μM) | CC₅₀ (μM) |
|---|---|---|---|
| Cholesterol | >30 | >30 | >30 |
| 4-beta-OHC | >30 | >30 | >30 |
| 7-KC | 14.5 | >30 | >30 |
| 22(S)-OHC | >30 | >30 | >30 |
| 22(R)-OHC | 13.2 | >30 | >30 |
| 24(S)-OHC | 1.3 | >30 | >30 |
| 27-OHC | 3.5 | >30 | >30 |
| RDV | 1.5 | 2.5 | >10 |
| Oxy133 | >15 | >15 | >15 |
| Oxy186 | 7.4 | >15 | >15 |
| Oxy210 | 5.5 | 8.6 | >15 |
| Oxy232 | 5.4 | 7.8 | >15 |

Semi-Synthetic Oxysterol Derivatives, Oxy210, Oxy186, and Oxy232 Inhibit SARS-CoV-2 Production: Although the natural oxysterols, 7-KC, 22(R)-OHC, 24(S)-OHC, and 27-OHC showed modest anti-SARS-CoV-2 activities, their physiological concentrations are far below μM ranges [21,22] in the circulation of healthy humans, suggesting their limited role, if any, in preventing viral infection under physiological conditions. In the search for oxysterols with improved antiviral activity, we evaluated the potential of semi-synthetic oxysterol derivatives for SARS-CoV-2 inhibition. SARS-CoV-2-induced CPE and virus propagation were blocked when treated with Oxy210 but not Oxy133 (FIGS. 9A and 9B, panels d and e). Quantification of SARS-CoV-2 RNA in the culture supernatant at 24 h post-inoculation also showed that Oxy210 and its structurally related derivatives, Oxy186 and Oxy232, reduced viral RNA level in a dose-dependent manner, while Oxy133 did not show antiviral activity up to 15 μM (FIG. 9C). The antiviral activity of Oxy186 was almost equivalent to that of the natural oxysterols shown earlier; the maximum reduction in viral RNA was 83% when used at 12 μM as compared to control (FIG. 9C, note that the viral RNA shown in logarithm scale). On the other hand, Oxy210 and Oxy232 showed much higher antiviral potencies; viral RNA production was reduced by 99.4% (Oxy210) and 99.9% (Oxy232) at the maximum at 15 μM (FIG. 9C). No significant cytotoxicity by Oxy186 and Oxy210 was found up to 15 μM, the maximum concentration in the infection assay; however, Oxy232 slightly reduced cell viability when used at concentrations above 10 μM (FIG. 9D). Due to the greater availability of Oxy210 we performed further studies with this oxysterol analog. The 50% and 90% maximal inhibitory concentration (IC₅₀, IC₉₀) and 50% maximal cytotoxic concentration (CC₅₀) of Oxy210 were 5.5 μM, 8.6 μM, and >15 μM (Table1), respectively.

Inventors previously reported that Oxy210 inhibited Hedgehog (Hh) and transforming growth factor β (TGFβ) signaling in fibroblastic cells and tumor cells [16]. In contrast, Oxy232, a close structural analog of Oxy210, is devoid of significant TGFβ inhibitory properties (FIG. 9E) but retains inhibitory activity toward Hh signaling (FIG. 9F), indicating that inhibition of TGFβ signaling is not responsible for the anti-SARS-CoV-2 activity. Consistent with this observation, treatment with the TGFβ signaling inhibitor, SB431542, did not significantly inhibit the production of viral RNA (FIG. 9G). In addition, the inactivation of the Hh pathway by either HPI-1 or GDC0449 did not decrease the viral RNA levels (FIG. 9G). These data suggest that Oxy210, Oxy232, and other antiviral oxysterol analogs, inhibit SARS-CoV-2 production independently of the inhibition of Hh or TGFβ signaling pathways.

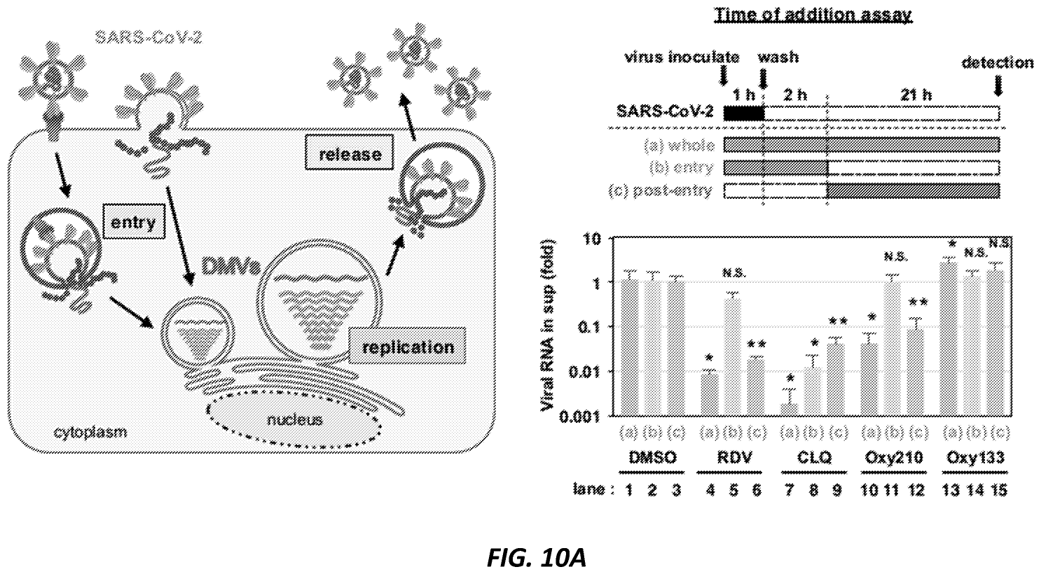

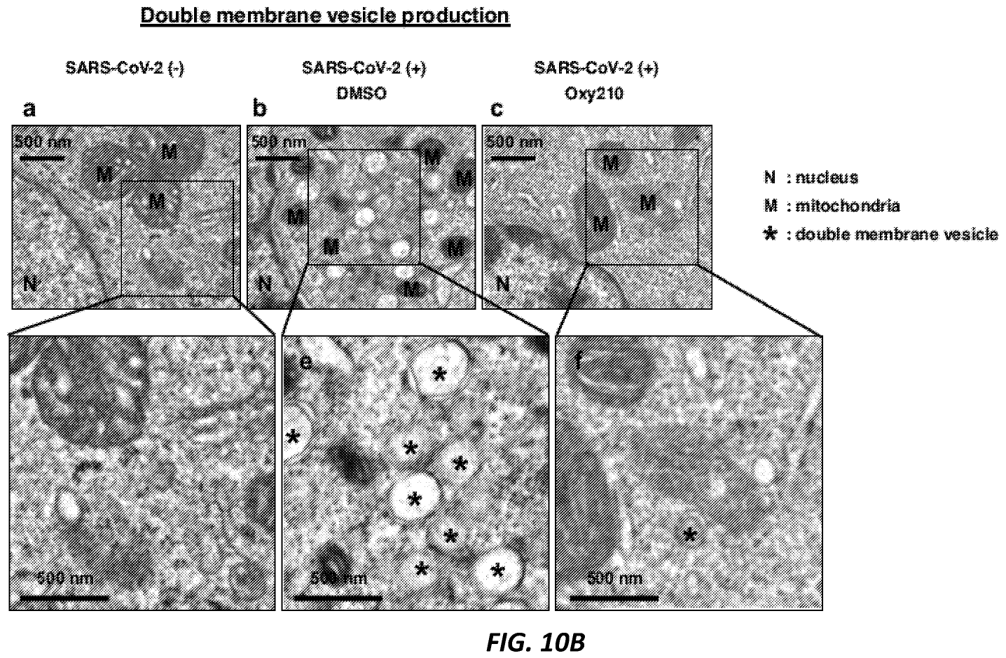

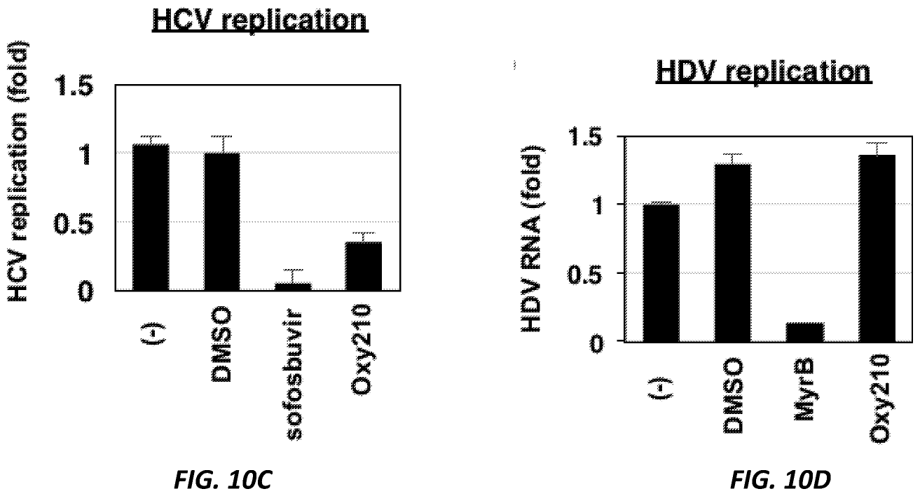

Oxy210 Inhibits the Intracellular SARS-CoV-2 Replication and Formation of Double Membrane Vesicles: To determine which steps in the SARS-CoV-2 life cycle (FIG. 10A, left) were inhibited by Oxy210, the inventors performed a time of addition assay (FIG. 10A, upper right). They examined the antiviral effect of Oxy210 in three different experimental groups, with different compound treatment times (FIG. 10A, a-c); (a) Compounds were treated during the 1 h virus inoculation and the additional 23 h up to detection to represent the whole life cycle (a, blue); (b) Compounds were present during the 1 h virus inoculation and an additional 2 h, and then removed to represent the virus entry process (b, green); and (c) Compounds were added 2 h after virus inoculation and were present for the remaining 21 h to represent the post-entry period (c, orange). The inventors confirmed that Chloroquine (CLQ), a reported SARS-CoV-2 entry inhibitor that acts through modulation of intracellular pH [2,23,24], showed the most inhibitory effect when introduced in the entry step of infection (FIG. 10A, lower right, lane 8). (Because of the multiple rounds of viral re-infection in the assay, entry inhibitors can also show antiviral effects when introduced at post-entry (FIG. 10A, lower right, lane 9)). The inventors also confirmed the mode of action of RDV, a reported inhibitor of intracellular viral RNA replication [25], by showing no significant effect on the virus entry-step (FIG. 10A, lower right, lane 5) and a remarkable inhibition of post-entry (FIG. 10A, lower right, lane 6). In this assay system, Oxy210, but not the negative control, Oxy133, clearly reduced viral RNA levels when present during the whole life cycle and the post-entry, but not at the entry phase, similar to the effects of RDV (FIG. 10A, lower right, lanes 10-12). This finding indicates that Oxy210 targets intracellular virus replication rather than viral entry.

Coronaviruses generally induce the formation of unique membrane compartments, called double-membrane vesicles (DMVs), which enable an efficient viral RNA replication [18,26]. The inventors found that DMV formation occurs with infection by SARS-CoV-2 in VeroE6/TMPRSS2 cells (FIG. 10B, panels b, e, *). Interestingly, treatment with Oxy210 remarkably reduced the DMV formation in the SARS-CoV-2-infected cells (FIG. 10B, panels c, f, *). They examined the specificity of Oxy210's effect on DMV-dependent virus replication by evaluating the antiviral effect on hepatitis C virus (HCV) and hepatitis D virus (HDV), which are other RNA viruses that drive replication in a DMV-dependent and -independent manner, respectively [26,27]. Similar to the effect of an HCV polymerase inhibitor, sofosbuvir, used as a positive control, Oxy210 reduced the DMV-dependent RNA replication of HCV (FIG. 10C), while the antiviral activity was not observed in HDV infection that was inhibited by the positive control, MyrB (FIG. 10D). These data are consistent with the idea that Oxy210 specifically inhibits the DMV-dependent virus replication.

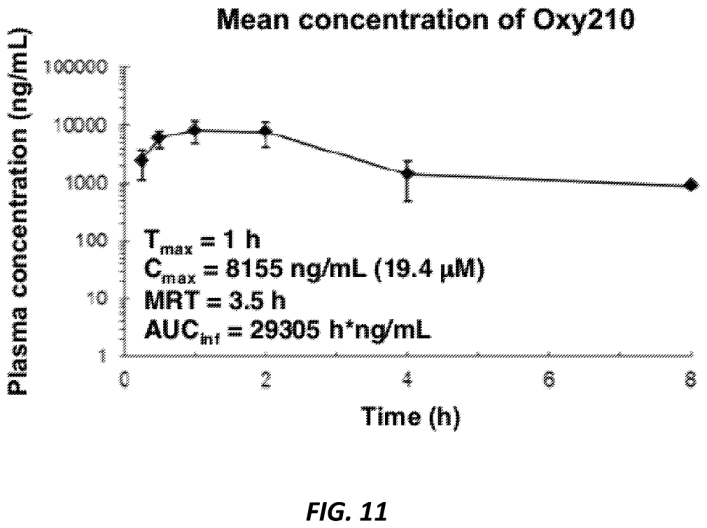

Pharmacokinetics of Oxy210 in Mice: Given its higher anti-SARS-CoV-2 potency compared to the natural oxysterols, whether oral administration of Oxy210 in mice would result in plasma concentrations high enough to sustain significant antiviral activity in vivo was studied. According to a previously established protocol [16], a single dose of Oxy210 at 200 mg/kg was orally administered to mice, and the plasma concentration was examined at 0.25, 0.5, 1, 2, 4, and 8 h (h). Oxy210 was well tolerated by the mice in this experiment. After 1 h (Tₘₐₓ), Oxy210 reached a peak plasma concentration (Cₘₐₓ) of 8,155 ng/mL (19.4 μM) with an overall exposure of 29,305 h*ng/mL, as measured by the area under the curve (AUC) (FIG. 11).

In a separate study, Oxy210 was administered to mice via a chow diet containing 4 mg Oxy210/g of food. Oxy210 plasma concentrations were measured at 24, 48, and 96 h. No adverse effects or significant loss of body weight was recorded during this 96 h experiment. Accumulation of Oxy210 in plasma was greatest after 96 h, averaging at 2,682 ng/mL (6.4 μM) (Table 2). The concentration in the liver and the lung after 96 h was higher at 6,869 ng/mL (16.3 μM) and 4,137 ng/mL (9.8 μM), respectively (Tables 3 and 4). These pharmacokinetic data indicate that oral administration of Oxy210 in mice, via oral gavage or mixed into a chow diet, results in plasma and lung concentrations that can sustain significant anti-SARS-CoV-2 activity in vivo.

TABLE 2
Plasma concentrations of Oxy210

| Time (h) | Sample concentration (ng/mL) | | | | | Mean ± SD |
|---|---|---|---|---|---|---|
| 24 | 26 | 267 | 8 | 12 | 83 | 79 ± 109 |
| 48 | 1039 | 652 | 1218 | 764 | 622 | 859 ± 259 |
| 96 | 1157 | 771 | 4471 | 6050 | 959 | 2682 ± 2423 |

TABLE 3
Liver concentrations of Oxy210

| Time (h) | Sample concentration (ng/g) | | | | | Mean ± SD |
|---|---|---|---|---|---|---|
| 96 | 5227 | 3470 | 7302 | 13291 | 5055 | 6869 ± 1717 |

TABLE 4
Lung concentrations of Oxy210

| Time (h) | Sample concentration (ng/g) | | | | | Mean ± SD |
|---|---|---|---|---|---|---|
| 96 | 1688 | 1109 | 6083 | 10524 | 1282 | 4137 ± 1843 |

FIG. 10 is a line graph showing pharmacokinetics of Oxy210 in mice. A single dose of Oxy210 at 200 mg/kg, formulated in 3% DMSO+7% Ethanol+5% PEG400+85% corn oil, was administered to balb/c mice by oral gavage. Plasma samples were taken at 0.25, 0.5, 1, 2, 4, and 8 h, followed by LC/MS analysis of the plasma to quantify Oxy210 concentrations.

### DISCUSSION

In this study, the inventors evaluated the anti-SARS-CoV-2 activity of a collection of naturally occurring and semi-synthetic oxysterol derivatives in cell cultures. Oxysterols are a class of understudied molecules that, until recently, have rarely been considered as a source of therapeutic drug candidates. In fact, most naturally occurring oxysterols cannot be ideal drug candidates for several different reasons, such as metabolic instability and overlapping biological activities. For example, 25-OHC, in addition to its antiviral properties, can also amplify the activation of immune cells and increases the production of potentially harmful immune mediators, which are linked to the development of atherosclerosis [5]. Semi-synthetic oxysterol derivatives, by contrast, often possess improved drug-like properties, in terms of potency, selectivity, metabolic stability, and drug safety characteristics, compared to their naturally occurring counterparts. Given the urgency of the COVID-19 pandemic, the inventors sought to establish a suitable drug development candidate with potent anti-SARS-CoV-2 activity that does not elicit unrelated or untoward pharmacological responses. In this study, Oxy210, a semi-synthetic oxysterol, was identified as a potent replication inhibitor of the SARS-CoV-2, which reduced the formation of DMVs. The peak plasma concentration of Oxy210 reached after administration via oral gavage (19 μM) and the plasma (6.4 μM) and lung (9.8 μM) concentrations reached after administration through diet, fall into a therapeutically meaningful range as they approach or exceed the IC₅₀ (5.5 μM) and IC₉₀ (8.6 μM) concentrations observed in the cell-based assays described herein. Therefore, Oxy210 and its analogs, such as Oxy232, can serve as drug candidates targeting COVID-19.

Inventors have previously characterized Oxy210 as a Hh and TGFβ signaling inhibitor [16] and have demonstrated protective effects of Oxy210 in a mouse model of idiopathic pulmonary fibrosis (IPF) (Parhami et al., unpublished observations). Oxy232, a close structural analog of Oxy210 but devoid of significant TGFβ pathway inhibitory properties, displayed anti-SARS-CoV-2 activity comparable to Oxy210, suggesting that the mechanisms of the anti-SARS-CoV-2 activity shared by Oxy210 and Oxy232 are likely unrelated to TGFβ inhibitory properties exhibited by Oxy210. This notion was further supported by the lack of antiviral activity displayed by a TGFβ pathway inhibitor, SB431542 (10 μM). Without wishing to bound by a theory, the antiviral activity is not likely to be due to the inhibition of the Hh pathway, as indicated by the lack of antiviral activity of Hh pathway inhibitors, HPI-1 (10 μM) and GDC0449 (10 μM). Given the absence of unrelated biological activities, such as TGFβ inhibition, Oxy232 can be a preferable drug candidate compared to Oxy210.

A recent publication reported the significant anti-SARS-CoV-2 activity of 27-OHC; low concentrations of 27-OHC inhibited post-entry, and higher concentrations inhibited the viral entry process [21]. The time-of-addition analysis data presented here suggest that Oxy210 predominantly inhibits the post-entry process, which includes viral RNA replication in the replication factory and the following assembly of progeny virus and its secretion. The inventors observed the formation of DMVs in SARS-CoV-2-infected cells, as previously reported [18,28]. DMVs, membrane compartments separated from the nuclease/protease-rich cytosol, are generally considered to be sites for efficient replication of genomic RNA of coronaviruses and of certain other RNA viruses, such as HCV [26]. DMVs are also very likely to be important in SARS-CoV-2 replication [17]. The data herein show that the production of DMVs, induced by SARS-CoV-2, was substantially reduced with Oxy210 treatment. Antiviral effects of Oxy210 were also observed during the replication of HCV, a virus that depends on DMVs for replication, but not with HDV, a virus that replicates independently of DMVs. These findings show that Oxy210 specifically reduces DMV-dependent virus replication.

It is noteworthy to outline the potential advantages of semi-synthetic oxysterols as anti-SARS-CoV-2 agents, compared to established antiviral compounds, such as RDV:
1) RDV has to be administered intravenously, most often in a hospital setting, whereas the oxysterols could potentially be dosed orally (via a pill or liquid gel). A safe and reliable oral medication could be administered at an earlier stage, for example, at the time of confirming a SARS-CoV-2 infection, and potentially benefit asymptomatic individuals and those at increased risk of infection who have close contact with infected individuals, including medical care workers, as a prophylactic treatment.
2) Oxysterols reprogram the host cell, interfering with the ability of the virus to use its machinery to replicate, reducing the likelihood of emerging drug resistance, and likely possess universal antiviral activity against SARS-CoV-2 mutant strains.
3) The scaling up and manufacturing of oxysterol-based drug candidates is expected to be straightforward and process friendly, especially when compared to the manufacturing process of RDV, which is rather difficult to prepare at scale.

As such, semi-synthetic oxysterol derivatives, such as Oxy210 and Oxy232 are good COVID-19 therapeutics used alone or in combination with other therapies currently FDA approved or under investigation, such as RDV, convalescent plasma, or antibody treatments.

### Materials and Methods

Compounds and the Synthesis of Oxysterol Derivatives: Commercially available oxysterols were obtained from Sigma Aldrich (St. Louis, MO, USA). Oxy133, Oxy186, and Oxy210 were prepared as previously described [12,15,16]. Oxy232 was prepared via a similar three-step synthesis described for Oxy186 and Oxy210, except for using ethyl magnesium bromide (instead of methyl magnesium bromide or methyl lithium) in step three, as described below. RDV was purchased from Chemscene (Monmouth Junction, NJ, USA); CLQ was purchased from Tokyo Chemical Industry (Tokyo, Japan); GDC-0449 was purchased from APExBIO (Boston, MA, USA), HPI-1 and SB-431542 were purchased from Cayman Chemical (Ann Arbor, MI, USA), sofosbuvir was purchased from MedChemExpress (Monmouth Junction, NJ, USA), and Myrcludex-B was synthesized by Scrum (Tokyo, Japan).

Synthesis and Molecular Characterization of semi-synthetic oxysterol derivatives Oxy133, Oxy186, Oxy210 and Oxy232: Materials were obtained from commercial suppliers and were used without further purification. Air or moisture sensitive reactions were conducted under an argon atmosphere using oven-dried glassware and standard syringe/septa techniques. The reactions were monitored on silica gel TLC plates under UV light (254 nm) followed by visualization with Hanessian's staining solution. Chromatographic purifications were performed using a Teledyne ISCO CombiFlash Rf automated chromatography system. NMR spectra were measured in CDCl3.

Oxy133 was synthesized according to the method shown in Scheme 1.

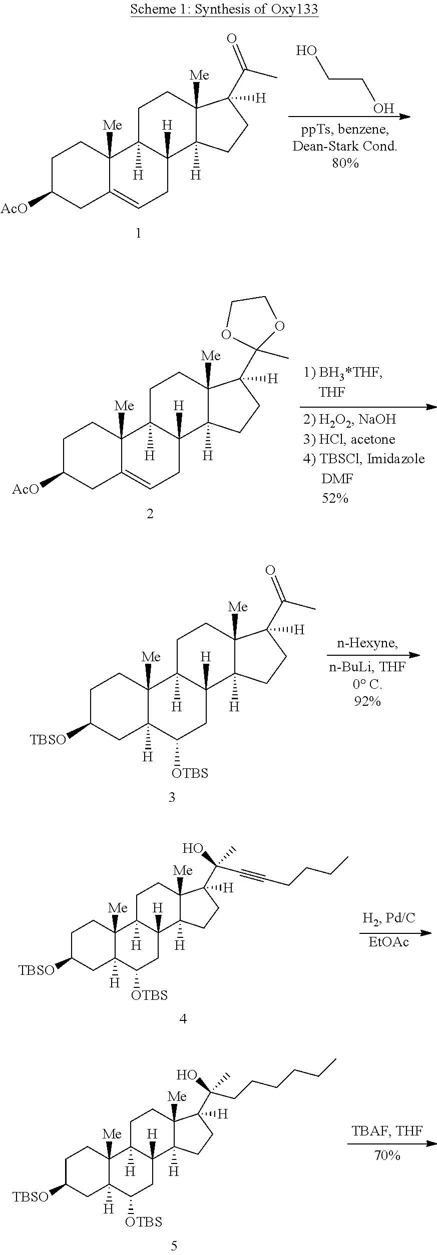

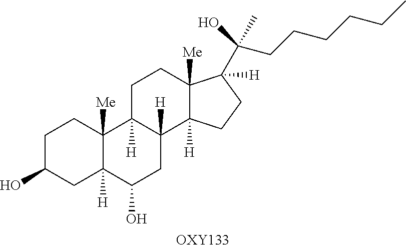

1-((3S,5S,6S,8R,9S,10R,13S,14S,17S)-3,6-bis((tert-butyldimethylsilyl)oxy)-10,13-dimethylhexadecahydro-1H-cyclopentalalphenanthren-17-yl)ethanone (3) was prepared according to a published patent procedure (Parhami, F.; Jung, M. E.; Nguyen, K.; Yoo, D.; Kim, W. WO 2009/07386, pp. 52). ¹H NMR (CDCl₃, 400 MHZ) δ: 3.47 (1H, dddd, J=11.0, 11.0, 4.8, 4.8 Hz), 3.36 (1H, ddd, J=10.4, 10.4, 4.4 Hz), 2.53 (1H, d, J=8.8, 8.8 Hz), 2.20-2.14 (1H, m), 2.10 (3H, s), 2.01-1.97 (1H, m), 1.88-1.82 (1H, m), 1.73-0.89 (17H, m), 0.88, 18H, s), 0.79 (3H, s), 0.59 (3H, s), 0.043 (3H, s), 0.04 (3H, s), 0.03 (3H, s), 0.02 (3H, s). ¹³C NMR (CDCl₃, 100 MHZ) δ: 209.5, 72.2, 70.1, 63.7, 56.4, 53.7, 51.8, 44.2, 41.9, 38.9, 37.6, 36.3, 34.3, 33.2, 31.7, 31.5, 25.94, 25.92, 24.4, 22.7, 21.1, 18.3, 18.1, 13.5, 13.4, −4.1, −4.6, −4.7.

(R)-2-((3S,5S,6S,8R,9S,10R,13S,14S,17S)-3,6-bis((tert-butyldimethylsilyl) oxy)-10,13-dimethylhexadecahydro-1H-cyclopentalalphenanthren-17-yl)oct-3-yn-2-ol (4): To a cold (0° C.) solution of n-hexyne (1.5 mL, 12 mmol) in THF (6 mL) was added a 1.6 M solution of n-BuLi in hexanes (3.75 mL). The resulting solution was stirred for 30 min until a solution of 1-((3S,5S,6S,8R,9S,10R,13S,14S,17S)-3,6-bis((tert-butyl dimethyl silyl) oxy)-10,13-dimethylhexadecahydro-1H-cyclopenta[a] phenanthren-17-yl)ethanone (3), (1.27 g, 2.2 mmol) in THF (10 mL) was added via cannula. The mixture was warmed to room temperature over 3 h and diluted with water (40 mL) and the crude product was isolated by ethyl acetate extraction (3×30 mL). The combined organic layers were washed with brine and dried over Na2SO4. Concentration gave an oily product which was purified on silica gel (hexane, EtOAc, gradient). There was 1.30 g of the product (4) (92%). ¹H NMR (CDCl₃, 300 MHZ) δ: 3.50 (1H, ddd, J=15.9, 11.0, 4.8 Hz), 3.36 (1H, dt, J=10.6, 4.3 Hz), 2.18 (1H, t, J=6.9 Hz), 2.10 (1H, m), 1.91-1.62 (4H, m), 1.53-1.31 (2H, m, 3H, s), 1.31-0.93 (22H, m), 0.93 (3H, s), 0.92 (3H, m), 0.90 (18H, s), 0.88 (3H, s), 0.61 (1H, m), 0.04 (6H, s), 0.03 (6H, s). ¹³C NMR (CDCl₃, 75 MHZ) δ: 85.9, 83.9, 72.4, 71.4, 70.3, 60.5, 55.8, 53.8, 51.8, 43.5, 36.3, 33.7, 33.0, 30.7, 25.9, 22.0, 18.4, 18.3, 18.1, 13.6, 13.5, −4.7, −4.7.

(S)-2-((3S,5S,6S,8R,9S,10R,13S,14S,17S)-3,6-bis((tert-butyldimethylsilyl)oxy)-10,13-dimethylhexadecahydro-1H-cyclopentalalphenanthren-17-yl)octan-2-ol (5): (R)-2-((3S,5S,6S,8R,9S,10R,13S,14S,17S)-3,6-bis((tert-butyldimethylsilyl)oxy)-10,13-dimethylhexadecahydro-1H-cyclopenta[a]phenanthren-17-yl)oct-3-yn-2-ol (4), (1.3 g, 2.0 mmol) was dissolved in EtOAc (5 mL), MeOH (5 mL) and Pd/C (10%, 0.1 g) was added to the solution. The mixture was degassed repeatedly under vacuum and then exposed to hydrogen gas under atmospheric pressure (balloon). After 18 h at room temperature, the mixture was diluted with EtOAc (20 mL) and filtered over Celite to remove the catalyst. The filter washed with EtOAc and the combined filtrates evaporated. There was 1.3 g of reduced product which was used without further purification. ¹H NMR (CDCl₃, 300 MHZ) δ: 3.50 (1H, ddd, J=15.9, 11.0, 4.8 Hz), 3.36 (1H, dt, J=10.6, 4.3 Hz), 2.1-1.95 (2H, m), 1.75-1.35 (10H, m), 1.32-1.29 (10H, m, 3H, s), 0.91-1.21 (10H, m), 0.89 (18H, s), 0.82 (3H, s), 0.79 (3H, s), 0.63 (3H, m), 0.04 (6H, s), 0.03 (6H, s)¹³C NMR (CDCl₃, 75 MHZ) δ: 75.2, 72.3, 57.6, 56.4, 53.8, 51.8, 42.9, 37.6, 36.3, 33.7, 31.9, 30.0, 25.9, 22.6, 18.3, 18.1, 14.1, 13.8, 13.5, −4.6, −4.7.

(3S,5S,6S,8R,9S,10R,13S,14S,17S)-17-((S)-2-hydroxyoctan-2-yl)-10,13-dimethyl hexadecahydro-1H-cyclopentalalphenanthrene-3,6-diol (Oxy133): A 1 M solution of TBAF in THF (8 mL, 8 mmol, 4 equiv) was directly added to (S)-2-((3S,5S,6S,8R,9S,10R,13S,14S,17S)-3,6-bis((tert-butyldimethylsilyl)oxy)-10,13-dimethyl hexadecahydro-1H-cyclopenta[a]phenanthren-17-yl)octan-2-ol (5), (1.3 g, 2.0 mmol, 1.0 equiv) and the resulting solution was diluted with THF (1 mL) and stirred at room temperature for 72 h. The mixture was then diluted with water (50 mL) and extracted repeatedly with EtOAc (4×40 mL). The combined organic layers were washed with brine, dried over Na2SO4 and the solvent evaporated. Purification of the crude product by silica gel chromatography (hexane, EtOAc, gradient, then 10% MeOH in EtOAc) afforded a white solid (0.6 g, 70%) which was subjected to trituration in aqueous acetone (acetone, water, 3:1). ¹H NMR (CDCl₃, 300 MHZ) δ: 3.50 (1H, ddd, J=15.9, 11.0, 4.8 Hz), 3.36 (1H, dt, J=10.6, 4.3 Hz), 2.19 (1H, m), 2.10-1.90 (3H, m), 1.85-1.60 (7H, m), 1.55-1.38 (7H, m), 1.25 (11H, brs), 1.20-0.95 (4H, m), 0.90 (3H, m), 0.86 (3H, s), 0.80 (3H, s) 0.62 (1H, m). ¹³C NMR (CDCl₃, 75 MHZ) δ: 75.1, 71.1, 69.3, 57.5, 56.2, 53.6, 51.6, 44.0, 42.8, 41.4, 40.1, 37.2, 36.2, 33.5, 32.1, 31.8, 30.9, 29.9, 26.3, 24.2, 23.6, 22.5, 22.2, 20.9, 14.0, 13.6, 13.3. MS: M+H=420.36. HRMS (ESI) m/z [M−2(H2O)+H]+ calcd for C27H44OH: 385.3470, found 385.3478.

(3S,5S,8R,9S,10S,13S,14S,17S)-17-((R)-4-(4-fluorophenyl)-2-hydroxybutan-2-yl)-10,13-dimethylhexadecahydro-1H-cyclopenta[a]phenanthren-3-ol (Oxy186) was synthesized according to the procedure shown in Scheme 2.

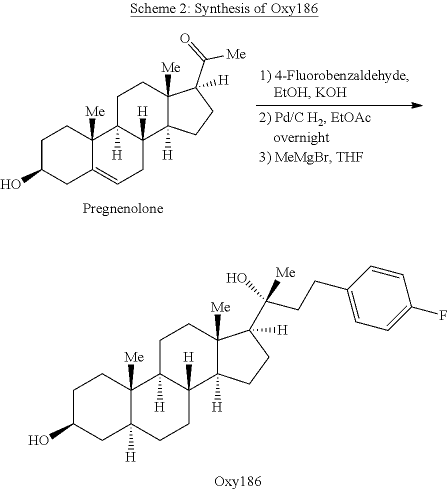

Oxy186 was prepared in three synthetic steps as shown above. Briefly, pregnenolone was condensed with 4-fluorobenzaldehyde to the enone which was reduced along with the C-5,6 double bond by hydrogenation using palladium on carbon (Pd/C) as a catalyst. The resulting fully saturated ketone was reacted with methylmagnesium bromide to afford Oxy186. The crude product was purified by chromatography on silica. ¹H NMR (CDCl₃, 400 MHZ) δ: 7.14-7.11 (2H, m), 6.97 (2H, dd, J=8.8, 8.8 Hz), 3.54 (1H, dddd, J=0.9, 10.9, 5.5, 5.5 Hz), 2.73-2.64 (2H, m), 2.32-0.63 (15H, m), 1.21 (3H, s), 0.80 (3H, s), 0.76 (3H, s). ¹³C NMR (CDCl₃, 100 MHZ) δ: 161.2 (d, J=242 Hz), 138.2 (d, J=3.1), 129.6 (d, J=20 Hz), 115.1 (d, J=20 Hz), 75.7, 71.3, 58.8, 56.7, 54.3, 44.9, 44.7, 43.3, 40.6, 38.15, 37.0, 35.5, 34.9, 32.0, 31.5, 29.6, 28.7, 23.8, 26.8, 23.7, 23.3, 21.1, 14.0, 12.3.

(3S,8S,9S,10R,13S,14S,17S)-17-((R)-2-hydroxy-4-(pyridin-3-yl)butan-2-yl)-10,13-dimethyl-2,3,4,7,8,9,10,11,12,13,14,15,16,17-tetradecahydro-1H-cyclopenta[a]phenanthren-3-ol (Oxy210) was synthesized according to the procedure shown in Scheme 3.

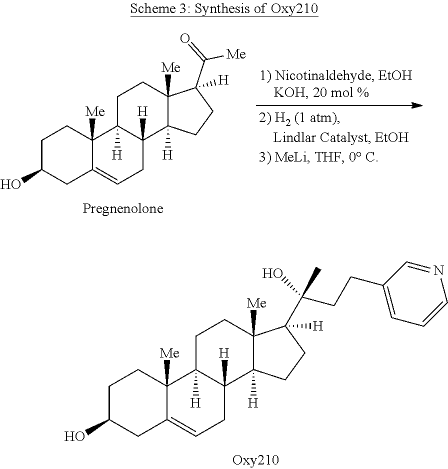

Oxy210 was prepared in three synthetic steps as shown above. Pregnenolone was condensed with nicotinaldehyde to the enone which was reduced via hydrogenation using Lindlar's catalyst. The saturated ketone was reacted with methyllithium to afford Oxy210. The crude product was purified by chromatography on silica. ¹H NMR (400 MHz, CDCl₃) δ 8.45 (1H, d, J=1 Hz), 8.42 (1H, dd, J=5, 2 Hz), 7.53-7.48 (1H, m), 7.23-7.18 (1H, m), 5.35-5.31 (1H, m), 3.56-3.45 (1H, m), 2.79-2.63 (2H, m), 2.33-2.17 (2H, m), 2.05 (1H, m), 2.01-1.26 (16H, m), 1.23 (3H, s), 1.18-0.89 (3H, m), 0.98 (3H, s), 0.87 (3H, s); ¹³C NMR (100 MHz, CDCl₃) δ 149.7, 147.1, 140.8, 138.1, 135.8, 128.6, 123.4, 121.4, 75.5, 71.6, 58.7, 56.9, 50.0, 44.1, 42.9, 42.3, 40.3, 37.2, 36.5, 31.7, 31.6, 31.3, 27.5, 26.7, 23.7, 23.2, 20.9, 19.3, 13.7.

(3S,5S,8R,9S,10S,13S,14S,17S)-17-((R)-3-hydroxy-1-(pyridin-3-yl)pentan-3-yl)-10,13-dimethylhexadecahydro-1H-cyclopenta[a]phenanthren-3-ol (Oxy232) was synthesized according to the procedure shown in Schemes 4-6.

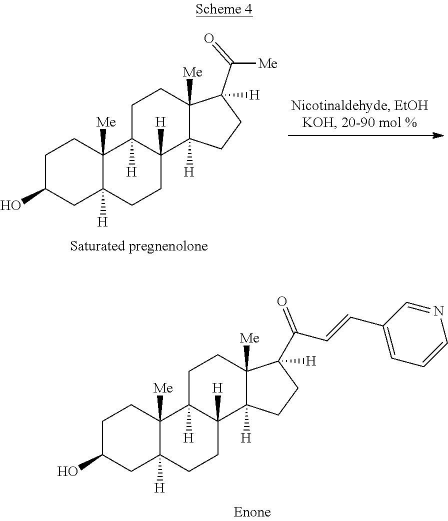

Step 1: Saturated pregnenolone (1.2 g, 3.8 mmol) was suspended in ethanol (20 mL) at room temperature. A potassium hydroxide solution (4M, 0.2 mL, 0.2 equivalents) was added to the reaction mixture followed by addition of nicotinaldehyde (0.7 g, 6.5 mmol). The resulting mixture was stirred at room temperature for 24 hours. Upon completion of the reaction (TLC analysis), water (50 mL) was added to the reaction mixture to precipitate the product. The crude solid product was isolated using vacuum filtration, washed with water (2×20 mL) and then air dried. There was obtained 1.56 g (>95%) of enone product. ¹H NMR (400 MHz, CDCl₃) δ 8.75 (1H, d, J=2 Hz), 8.57 (1H, dd, J=5, 2 Hz), 7.86-7.81 (1H, m), 7.50 (1H, d, J=17 Hz), 7.31 (1H, dd, J=8, 4 Hz), 6.81 (1H, d, J=17 Hz), 3.56-3.40 (1H, m), 2.83 (1H, dd, J=9, 9 Hz), 2.39-2.17 (3H, m), 2.06-1.95 (3H, m), 1.87-1.01 (14H, m), 0.81 (3H, s), 0.62 (3H, s).

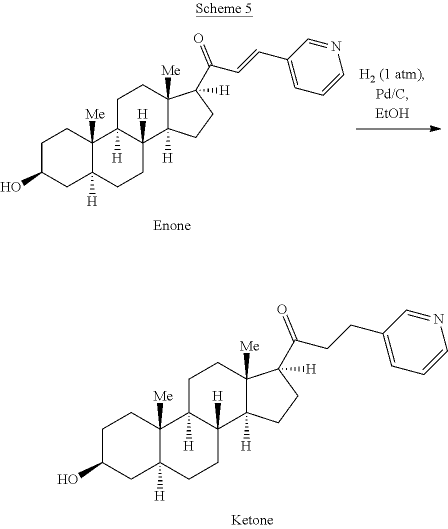

Step 2: The enone (1.5 g, 3.8 mmol) was suspended in ethanol (25 mL) and ethyl acetate (5 ml) at room temperature and palladium on carbon catalyst (0.15 g) was added to the mixture. The atmosphere in the reaction flask was purged three times with hydrogen gas using a balloon. The reaction mixture was then stirred at room temperature under a hydrogen atmosphere. After 2 days, the mixture was filtered over celite and concentrated in vacuo. The crude product mixture was purified via automated chromatography (ISCO) running an hexanes/ethyl acetate gradient (0-100%) to yield pure ketone product (1.0 g, 66%). ¹H NMR (400 MHz, CDCl₃) δ 8.42 (1H, d, J=2 Hz), 8.40 (1H, dd, J=5.1 Hz), 7.52-7.47 (1H, m), 7.17 (1H, dd, J=8, 5 Hz), 3.54-3.44 (1H, m), 2.92-2.91 (2H, m), 2.72-2.64 (2H, m), 2.45, (1H, dd, J=9, 9 Hz), 2.35-1.00 (17H, m), 0.% (3H, s), 0.52 (3H, s); ¹³C NMR (125 MHz, CDCl₃) δ 209.9, 149.8, 147.5, 136.9, 136.2 123.3, 71.2, 63.2, 56.8, 54.2, 45.3, 44.8, 44.6, 39.2, 38.1, 37.0, 35.5, 32.0 31.5, 28.6, 26.8 24.4, 23.0, 21.0, 13.3, 12.3.

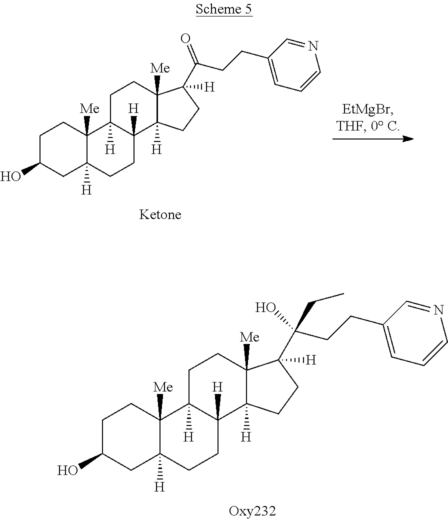

Step 3: The ketone (0.41 g, 1 mmol) was dissolved in dry tetrahydrofuran (5 mL) at room temperature and cooled to 0° C. under N2-atmosphere. A solution of ethyl magnesium bromide (3 M in ether, 2 mL, 6 mmol) was added dropwise to the reaction mixture at 0° C. The reaction mixture was then stirred at 0° C. for 1 hour until the starting material was mostly consumed (TLC analysis). Then the reaction was carefully quenched with a small volume (˜1 mL) of methanol and the mixture further diluted with saturated ammonium chloride solution (20 mL) and dichloromethane (20 mL). The layers were separated, and the aqueous layer extracted with dichloromethane (2×30 mL). The combined organic layers were dried over sodium sulfate, filtered and concentrated in vacuo. The crude mixture was purified via automated chromatography (ISCO) running an ethyl acetate/methanol gradient (0-10%) to yield Oxy232 (0.35 g, 80%). ¹H NMR (400 MHz, CDCl₃) δ 8.42 (1H, d, J=1 Hz), 8.38 (1H, dd, J=5, 2 Hz), 7.53-7.48 (1H, m), 7.23-7.18 (1H, m), 3.54 (1H, m), 2.69-2.63 (2H, m), 2.33-2.17 (2H, m), 2.05 (1H, m), 2.01-1.26 (17H, m), 1.22 (2H, m), 1.18-0.60 (6H, m), 0.85 (3H, s), 0.77 (3H, s); ¹³C NMR (100 MHz, CDCl₃) δ 149.1, 146.6, 138.5, 136.4, 123.6, 77.3, 71.2, 56.8, 55.3, 54.3, 44.9, 43.0, 40.7, 38.8, 38.2, 37.0, 35.4, 34.8, 32.0, 31.5, 31.0, 28.7, 27.3, 23.6, 22.3, 21.1, 13.8, 12.3, 8.4.

Cell Culture: VeroE6/TMPRSS2 cells, VeroE6 cells overexpressing transmembrane protease, serine 2 (TMPRSS2) [20,29], were cultured in Dulbecco's modified Eagle's medium (DMEM; Wako, Osaka, Japan) supplemented with 10% fetal bovine serum (FBS; Sigma Aldrich, St. Louis, MO, USA), 100 units/mL penicillin, 100 μg/mL streptomycin, 10 mM HEPES (pH 7.4), and 1 mg/mL G418 (Nacalai, Kyoto, Japan) at 37° C. in 5% CO₂. During the infection assay, 10% FBS was replaced with 2% FBS, and G418 removed. LucNeo #2 cells, carrying HCV subgenomic replicon, were kindly provided by Kunitada Shimotohno at National Center for Global Health and Medicine [30] and were cultured in DMEM supplemented with 10% FBS, 10 units/mL penicillin, 10 μg/mL streptomycin, 0.1 mM non-essential amino acids (Invitrogen, Carlsbad, CA, USA), 1 mM sodium pyruvate, 10 mM HEPES (pH 7.4), and 0.5 mg/mL G418 at 37° C. in 5% CO₂. HepG2-hNTCP-C4 cells, a HepG2 cell clone overexpressing the HDV entry receptor, sodium taurocholate cotransporting polypeptide (NTCP), and highly susceptible to HDV infection [6] were cultured in GlutaMax (Invitrogen, Carlsbad, CA, USA) supplemented with 100 units/mL penicillin, 100 μg/mL streptomycin, 10% FBS, 10 mM HEPES (pH 7.4), 50 μM hydrocortisone, and 5 μg/mL insulin at 37° C. in 5% CO₂.

SARS-CoV-2 Infection Assay: SARS-CoV-2 was handled in a biosafety level 3 (BSL3) facility. We used the SARS-CoV-2 Wk-521 strain, a clinical isolate from a COVID-19 patient, and obtained viral stocks by infecting VeroE6/TMPRSS2 cells [20]. VeroE6/TMPRSS2 cells were inoculated with SARS-CoV-2 at an MOI of 0.001 (FIGS. 8B, 8C, 9A and 9B), 0.003 (FIGS. 8D, 9C, and 10A), and 1 (FIG. 10B) for 1 h and unbound virus removed by washing. Cells were cultured for 24 h prior to measuring extracellular viral RNA or detecting virally encoded N protein, for 48 h to detect cytopathic effects (CPE), and for 7 h to observe cells by electron microscopy. Compounds were added during virus inoculation (1 h) and after washing (24 or 48 h) except for the time of addition assay shown in FIG. 10A.

For the time of addition assay, we added compounds with three different timings (FIG. 10A): (a) present during the 1 h virus inoculation and maintained throughout the 23 h infection period (whole life cycle); (b) present during the 1 h virus inoculation and for an additional 2 h and then removed (entry); or (c) added at 2 h after virus inoculation and present for the remaining 21 h until harvest (post-entry). Inhibitors of viral replication such as remdesivir (RDV) were expected to show antiviral activity in (a) and (c), but not (b), while entry inhibitors including CLQ reduce viral RNA in all three conditions [2].

Quantification of Viral RNA: Viral RNA in the culture supernatant was extracted with a QIAamp Viral RNA mini (QIAGEN, Venlo, Netherlands) or MagMax Viral/Pathogen II Nucleic Acid Isolation kit (Thermo Fisher Scientific, Waltham, MA, USA) and quantified by real-time RT-PCR analysis with a one-step qRT-PCR kit (THUNDERBIRD Probe One-step qRT-PCR kit, TOYOBO, Osaka, Japan) using 5′-ACAGGTACGTTAATAGTTAATAGCGT-3′ (SEQ ID NO: 7), 5′-ATATTGCAGCAGTACGCACACA-3′ (SEQ ID NO: 8) and 5′-FAM-ACACTAGCCATCCTTACTGCGCTTCG-TAMRA-3′ (E-set) (SEQ ID NO: 9) [31].

Detection of Viral N Protein: The viral N protein was detected using a rabbit anti-SARS-CoV N antibody [32] as a primary antibody with AlexaFluor 568 anti-rabbit IgG or anti-rabbit IgG-HRP (Thermo Fisher, Waltham, MA, USA) as secondary antibodies together with DAPI to stain the nucleus by indirect immunofluorescence as described previously [33].

Quantification of Cell Viability: Cell viability was determined by MTT assay as previously reported [33].

Quantification of Transforming Growth Factor, β (TGFβ) and Hedgehog (Hh) Activity: TGFβ activity was examined with NIH3T3 cells precultured with DMEM containing 0.1% bovine calf serum (BCS) overnight. NIH3T3 cells were pretreated with the compounds for 2 h and then stimulated with TGFβ1 (20 ng/mL) in the presence or absence of compounds. After 48 h, RNA was extracted from the cells and analyzed for quantifying the mRNA for a TGFβ target gene, connective tissue growth factor (CTGF), and Oaz1 for normalization. For examination of Hh activity, NIH3T3 cells pretreated with the compounds for 2 h were treated with conditioned medium from CAPAN-1 human pancreatic tumor cells that contain Shh in the absence or presence of the compounds. Cellular RNA was extracted and analyzed for the expression of a Hh target gene, Gli1, and normalized to Oaz1 expression.

Electron Microscopic Analysis: Cells were fixed with the buffer (2.5% glutaraldehyde, 2% paraformaldehyde, and 0.1 M phosphate buffer (pH 7.4)) for 1 h at room temperature followed by with 1% osmium tetroxide, stained in 1% uranyl acetate, dehydrated through a graded series of alcohols and embedded in Epon. Ultrathin sections were stained with uranyl acetate and lead citrate to observe with a transmission electron microscope (HT7700; Hitachi, Ltd., Tokyo, Japan)

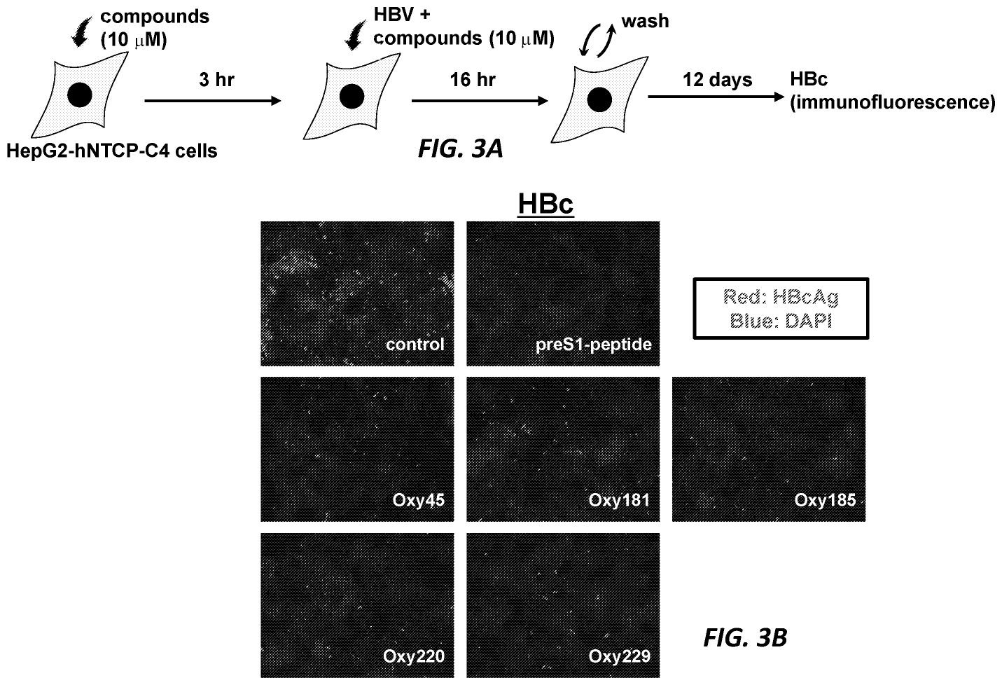

Hepatitis C Virus (HCV) Replication Assay: HCV replication activity was measured using LucNeo #2 cells, carrying a subgenomic replicon RNA for an HCV NN strain (genotype-1b) and the luciferase gene driven by the HCV replication [30]. LucNeo #2 cells were treated with the compounds indicated in FIG. 3C for 48 h, and the luciferase activity was measured with Luciferase Assay System kit (Promega, Madison, WI, USA). Sofosbuvir, a clinically used HCV polymerase inhibitor, was used as a positive control.

Hepatitis D Virus (HDV) Replication Assay: HDV was recovered from the culture supernatant of Huh7 cells transfected with the plasmids for HDV genome and for hepatitis B virus surface antigen [34]. HepG2-hNTCP-C4 cells were inoculated with HDV for 16 h and were further cultured for 6 days in the presence or absence of Oxy210 to detect intracellular HDV RNA [34]. Myrcludex-B (Myr-B), used as a positive control that inhibits HDV infection, was treated during the virus inoculation.

Pharmacokinetics of Oxy210 in Mice: We performed the pharmacokinetic analysis in mice by oral administration with Oxy210 was performed as described previously [16].

Institutional Review Board Statement: The mouse studies were conducted according to the guidelines of Pharmacology Discovery Services Taiwan, Ltd., a Eurofins Discovery Partner Lab. All aspects of this work, including housing, experimentation, and animal disposal were performed in general accordance with the “Guide for the Care and Use of Laboratory Animals: Eighth Edition” (National Academies Press, Washington, D.C., 2011) in an AAALAC-accredited laboratory animal facility. In addition, the animal care and use protocol was reviewed and approved by the IACUC at Pharmacology Discovery Services Taiwan, Ltd.

### REFERENCES

1. Watashi, K. Identifying and repurposing antiviral drugs against severe acute respiratory syndrome coronavirus 2 with in silico and in vitro approaches. Biochem. Biophys. Res. Commun. 2020, 538, 137-144.
2. Wang, M.; Cao, R.; Zhang, L.; Yang, X.; Liu, J.; Xu, M.; Shi, Z.; Hu, Z.; Zhong, W.; Xiao, G. Remdesivir and chloroquine effectively inhibit the recently emerged novel coronavirus (2019-nCoV) in vitro. Cell Res. 2020, 30, 269-271.
3. Edwards, A. What Are the Odds of Finding a COVID-19 Drug from a Lab Repurposing Screen? J. Chem. Inf. Model. 2020, 60, 5727-5729.
4. Griffiths, W. J.; Wang, Y. Oxysterol research: A brief review. Biochem. Soc. Trans. 2019, 47, 517-526.
5. Luchetti, F.; Crinelli, R.; Cesarini, E.; Canonico, B.; Guidi, L.; Zerbinati, C.; Di Sario, G.; Zamai, L.; Magnani, M.; Papa, S.; et al. Endothelial cells, endoplasmic reticulum stress and oxysterols. Redox Biol. 2017, 13, 581-587.
6. Iwamoto, M.; Watashi, K.; Tsukuda, S.; Aly, H. H.; Fukasawa, M.; Fujimoto, A.; Suzuki, R.; Aizaki, H.; Ito, T.; Koiwai, O.; et al. Evaluation and identification of hepatitis B virus entry inhibitors using HepG2 cells overexpressing a membrane transporter NTCP. Biochem. Biophys. Res. Commun. 2014, 443, 808-813.
7. Cagno, V.; Civra, A.; Rossin, D.; Calfapietra, S.; Caccia, C.; Leoni, V.; Dorma, N.; Biasi, F.; Poli, G.; Lembo, D. Inhibition of herpes simplex-1 virus replication by 25-hydroxycholesterol and 27-hydroxycholesterol. Redox Biol. 2017, 12, 522-527.
8. Civra, A.; Cagno, V.; Donalisio, M.; Biasi, F.; Leonarduzzi, G.; Poli, G.; Lembo, D. Inhibition of pathogenic non-enveloped viruses by 25-hydroxycholesterol and 27-hydroxycholesterol. Sci. Rep. 2014, 4, 7487.
9. Shawli, G. T.; Adeyemi, O. O.; Stonehouse, N. J.; Herod, M. R. The Oxysterol 25-Hydroxycholesterol Inhibits Replication of Murine Norovirus. Viruses 2019, 11, 97.
10. Civra, A.; Francese, R.; Gamba, P.; Testa, G.; Cagno, V.; Poli, G.; Lembo, D. 25-Hydroxycholesterol and 27-hydroxycholesterol inhibit human rotavirus infection by sequestering viral particles into late endosomes. Redox Biol. 2018, 19, 318-330.
11. Li, C.; Deng, Y.-Q.; Wang, S.; Ma, F.; Aliyari, R.; Huang, X.-Y.; Zhang, N.-N.; Watanabe, M.; Dong, H.-L.; Liu, P.; et al. 25-Hydroxycholesterol Protects Host against Zika Virus Infection and Its Associated Microcephaly in a Mouse Model. Immunity 2017, 46, 446-456.
12. Montgomery, S. R.; Nargizyan, T.; Meliton, V.; Nachtergaele, S.; Rohatgi, R; Stappenbeck, F.; Jung, M. E.; Johnson, J. S.; Aghdasi, B.; Tian, H.; et al. A Novel Osteogenic Oxysterol Compound for Therapeutic Development to Promote Bone Growth: Activation of Hedgehog Signaling and Osteogenesis Through Smoothened Binding. J. Bone Miner. Res. 2014, 29, 1872-1885.
13. Scott, T. P.; Phan, K. H.; Tian, H.; Suzuki, A.; Montgomery, S. R.; Johnson, J. S.; Atti, E.; Tetratis, S.; Pereira, R. C.; Wang, J. C.; et al. Comparison of a novel oxysterol molecule and rhBMP2 fusion rates in a rabbit posterolateral lumbar spine model. Spine J. 2015, 15, 733-742.
14. Buser, Z.; Drapeau, S.; Stappenbeck, F.; Pereira, R. C.; Parhami, F.; Wang, J. C. Effect of Oxy133, an osteogenic oxysterol, on new bone formation in rat two-level posterolateral fusion model. Eur. Spine J. 2017, 26, 2763-2772.
15. Wang, F.; Stappenbeck, F.; Parhami, F. Inhibition of Hedgehog Signaling in Fibroblasts, Pancreatic, and Lung Tumor Cells by Oxy186, an Oxysterol Analogue with Drug-Like Properties. Cells 2019, 8, 509.
16. Stappenbeck, F.; Wang, F.; Tang, L.-Y.; Zhang, Y. E.; Parhami, F.; Wang; Tang Inhibition of Non-Small Cell Lung Cancer Cells by Oxy210, an Oxysterol-Derivative that Antagonizes TGFβ and Hedgehog Signaling. Cells 2019, 8, 1297.
17. Du Toit, A. Coronavirus replication factories. Nat. Rev. Microbiol. Genet. 2020, 18, 411.
18. Wolff, G.; Limpens, R. W. A. L.; Zevenhoven-Dobbe, J. C.; Laugks, U.; Zheng, S.; De Jong, A. W. M.; Koning, R. I.; Agard, D. A.; Grunewald, K.; Koster, A. J.; et al. A molecular pore spans the double membrane of the coronavirus replication organelle. Science 2020, 369, 1395-1398.
19. Blanchard, E.; Roingeard, P. Virus-induced double-membrane vesicles. Cell. Microbiol. 2015, 17, 45-50.
20. Matsuyama, S.; Nao, N.; Shirato, K.; Kawase, M.; Saito, S.; Takayama, I.; Nagata, N.; Sekizuka, V.O.R.C.I.D.P.; Katoh, V.O.R.C.I.D.P.; Kato, F. Enhanced isolation of SARS-CoV-2 by TMPRSS2-expressing cells. Proceedings of the National Academy of Sciences 2020, 117, 7001-7003.
21. Marcello, A.; Marcelloa, A.; Civra, A.; Bonottoa, R. M.; Alves, L. N.; Rajasekharan, S.; Giacobone, C.; Caccia, C.; Cavalli, R.; Adami, M.; et al. The cholesterol metabolite 27-hydroxycholesterol inhibits SARS-CoV-2 and is markedly decreased in COVID-19 patients. Redox. Biol. 2020, 36, 101682.
22. Arca, M.; Natoli, S.; Micheletta, F.; Riggi, S.; Di Angelantonio, E.; Montali, A.; Antonini, T. M.; Antonini, R.; Diczfalusy, U.; Iuliano, L. Increased plasma levels of oxysterols, in vivo markers of oxidative stress, in patients with familial combined hyperlipidemia: Reduction during atorvastatin and fenofibrate therapy. Free. Radic. Biol. Med. 2007, 42, 698-705.
23. Akpovwa, H. Chloroquine could be used for the treatment of filoviral infections and other viral infections that emerge or emerged from viruses requiring an acidic pH for infectivity. Cell Biochem. Funct. 2016, 34, 191-1%.
24. Liu, J.; Cao, R.; Xu, M.; Wang, X.; Zhang, H.; Hu, H.; Li, Y.; Hu, Z.; Zhong, W.; Wang, M. Hydroxychloroquine, a less toxic derivative of chloroquine, is effective in inhibiting SARS-CoV-2 infection in vitro. Cell Discov. 2020, 6, 16.
25. Kokic, G.; Hillen, H. S.; Tegunov, D.; Dienemann, C.; Seitz, F.; Schmitzova, J.; Farnung, L.; Siewert, A.; Höbartner, C.; Cramer, P. Mechanism of SARS-CoV-2 polymerase stalling by remdesivir. Nat. Commun. 2021, 12, 279.
26. Paul, D.; Bartenschlager, R. Architecture and biogenesis of plus-strand RNA virus replication factories. World J. Virol. 2013, 2, 32-48.
27. Zhang, Z.; Urban, S. New insights into HDV persistence: The role of interferon response and implications for upcoming novel therapies. J. Hepatol. 2021, 74, 686-699.
28. Ogando, N. S.; Dalebout, T. J.; Zevenhoven-Dobbe, J. C.; Limpens, R. W.; Van Der Meer, Y.; Caly, L.; Druce, J.; De Vries, J. J. C.; Kikkert, M.; Bárcena, M.; et al. SARS-coronavirus-2 replication in Vero E6 cells: Replication kinetics, rapid adaptation and cytopathology. J. Gen. Virol. 2020, 101, 925-940.
29. Nao, N.; Sato, K.; Yamagishi, J.; Tahara, M.; Nakatsu, Y.; Seki, F.; Katoh, H.; Ohnuma, A.; Shirogane, Y.; Hayashi, M.; et al. Consensus and variations in cell line specificity among human metapneumovirus strains. PLoS ONE 2019, 14, e0215822.
30. Goto, K.; Watashi, K.; Murata, T.; Hishiki, T.; Hijikata, M.; Shimotohno, K. Evaluation of the anti-hepatitis C virus effects of cyclophilin inhibitors, cyclosporin A, and NIM811. Biochem. Biophys. Res. Commun. 2006, 343, 879-84.
31. Corman, V. M.; Landt, O.; Kaiser, M.; Molenkamp, R.; Meijer, A.; Chu, D. K.; Bleicker, T.; Briinink, S.; Schneider, J.; Schmidt, M. L.; et al. Detection of 2019 novel coronavirus (2019-nCoV) by real-time RT-PCR. Eurosurveillance 2020, 25, 23-30.
32. Mizutani, T.; Fukushi, S.; Saijo, M.; Kurane, I.; Morikawa, S. Phosphorylation of p38 MAPK and its downstream targets in SARS coronavirus-infected cells. Biochem. Biophys. Res. Commun. 2004, 319, 1228-1234.
33. Ohashi, H.; Nishioka, K.; Nakajima, S.; Kim, S.; Suzuki, R.; Aizaki, H.; Fukasawa, M.; Kamisuki, S.; Sugawara, F.; Ohtani, N.; et al. The aryl hydrocarbon receptor-cytochrome P450 1A1 pathway controls lipid accumulation and enhances the permissiveness for hepatitis C virus assembly. J. Biol. Chem. 2018, 293, 19559-19571.
34. Kaneko, M.; Watashi, K.; Kamisuki, S.; Matsunaga, H.; Iwamoto, M.; Kawai, F.; Ohashi, H.; Tsukuda, S.; Shimura, S.; Suzuki, R; et al. A Novel Tricyclic Polyketide, Vanitaracin A, Specifically Inhibits the Entry of Hepatitis B and D Viruses by Targeting Sodium Taurocholate Cotransporting Polypeptide. J. Virol. 2015, 89, 11945-11953.

### Example 4: Anti-SARS-CoV-2 Activity of Oxy232 and Oxy210 and in Vitro Properties of Oxy232

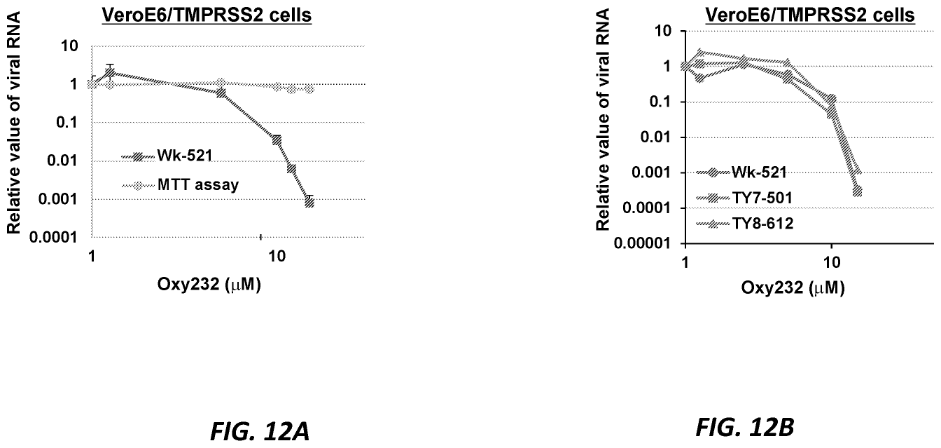

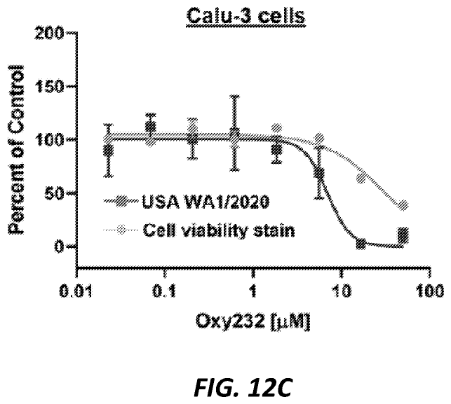

According to a previously published report (Ohashi et al., Identification of Anti-Severe Acute Respiratory Syndrome-Related Coronavirus 2 (SARS-CoV-2) Oxysterol Derivatives In Vitro. Int J Mol Sci. 2021; 22(6):3163. PMCID: PMC8003796) inventors have tested a collection of naturally occurring and semisynthetic oxysterol derivatives in a SARS-CoV-2 cell culture infection assay in VeroE6/TMPRSS2 cells. In the VeroE6/TMPRSS2 cell assay, Oxy210 displayed robust anti-SARS-CoV-2 activity with far greater suppression of viral replication at concentrations above 5 μM compared to all naturally occurring oxysterols tested (Ohashi et al., Int J Mol Sci. 2021; 22(6):3163). Oxy232, a close relative of Oxy210, also displayed significant anti SARS-CoV-2 activity, achieving viral inhibition greater than 99%, when tested in multiple studies, consistently trending toward greater suppression of viral replication compared to Oxy210 (FIG. 12A). The inventors have compared Oxy210 and Oxy232 against different viral strains of SARS-CoV-2 in VeroE6/TMPRSS2 cells, such as the Wk-521 (original Wuhan strain), TY7-501 (Brazil strain), and the TY8-612 (South Africa strain) and observed undiminished activity against the TY7-501 and TY8-612 strains (FIG. 12B). They have also confirmed the antiviral activity of Oxy232 in Calu-3 human lung epithelial cells at NIAID using the USA WA1/2020 isolate of SARS-CoV-2 (FIG. 12C). At higher concentrations, Oxy210 and Oxy232 do affect cell viability in VeroE6/TMPRSS2 and Calu-3 cells, as measured by MTT assay or automated microscopy (FIGS. 12A and 12C, Table 5). See, Ohashi et al., Int J Mol Sci. 2021; 22(6):3163 and Dittmar et al. Drug repurposing screens reveal cell-type-specific entry pathways and FDA-approved drugs active against SARS-Cov-2. Cell Rep. 2021; 35(1):108959. PMCID: PMC7985926. The data show excellent in vivo safety and tolerability in mice for Oxy210 and to a lesser degree for Oxy232. Without wishing to be bound by a theory, biological activities of oxysterols, including measures of cell viability, can heavily depend on in vitro assay conditions, such as the fetal bovine serum concentration or the duration of pretreatment with the compounds, among other factors, that may not translate to the in vivo safety of the compounds. The data presented in FIGS. 12A-12C and Table 5 show that the anti-SARS-CoV-2 activity of the oxysterols is not significantly affected by cell type or viral strain, consistent with a host-directed mechanism of action.

TABLE 5
IC₅₀, IC₉₀ and CC₅₀ values for Oxy210 and Oxy232 in VeroE6 and calu-3 cells

| Compound | IC₅₀ (VeroE6) | IC₉₀ (VeroE6) | CC₅₀ (VeroE6) | IC₅₀ (Calu-3) | | IC₉₀ (Calu-3) | CC₅₀ (Calu-3) | |
|---|---|---|---|---|---|---|---|---|
| Remdesivir | 1.5 μM | 1.5 μM | >10 μM | 0.08 | μM | 0.16 μM | >10 | μM |
| Oxy210 | 5.5 μM | 8.6 μM | >15 μM | 1.5 | μM | 4.24 μM | 8.3 | μM |
| Oxy232 | 5.4 μM | 7.8 μM | >15 μM | 6.9 | μM | 13.5 μM | 29.4 | μM |

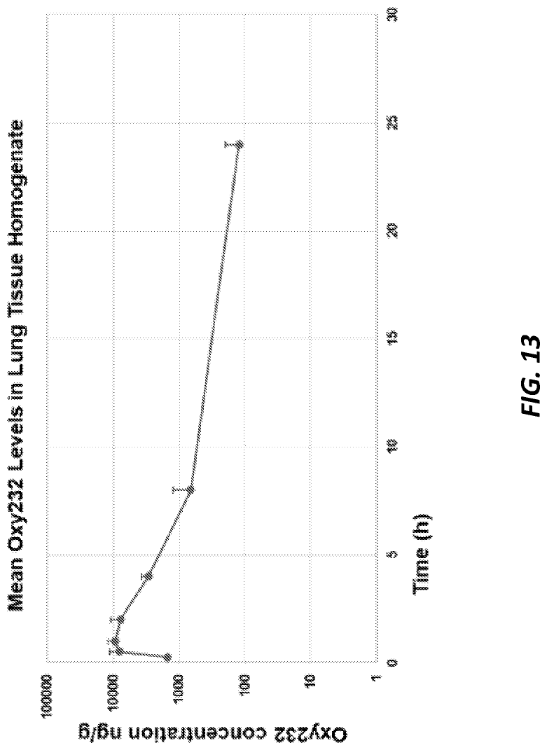

In preparation for in vivo efficacy studies using mouse models of Covid-19, available at NIAID and NIID (Japan), mouse plasma and lung exposure levels for Oxy210 and Oxy232 were analyzed. After a single dose of Oxy232 at 200 mg/kg in mice, plasma concentrations for Oxy232 above IC₅₀ levels for antiviral activity (VeroE6/TMPRSS2) for more than 3 hours and at IC₉₀ levels for 1.5 hours were observed. Oxy232 concentrations in lung tissue homogenate were generally higher than in plasma and above IC₅₀ levels for antiviral activity (VeroE6/TMPRSS2) for 5 hours and above IC₉₀ levels (VeroE6/TMPRSS2) for 3 hours (FIG. 13 and Table 6). The Oxy232 concentration in lung homogenate is above VeroE6 IC₅₀ levels (2374 ng/m for 5 h and above VeroE6 IC₉₀ levels (3249 ng/mL) for 4 h. The lung concentration of Oxy232 in lung homogenate is above Calu-3 IC₅₀ levels (3051 ng/L) for more than 3.5 h and above Calu-3 IC₉₀ levels (5918 ng/mL) for 2.5 h. Without wishing to be bound by a theory, twice daily dosing of Oxy232 at 200 mg/kg in mice, repeated over several days to allow for drug accumulation, would very likely result in significant and therapeutically meaningful lung exposure. The data show that sustained exposure of Oxy232 (plasma and lung) in excess of the IC₉₀ concentrations in VeroE6/TMPRSS2 cells and Calu-3 cells can be safely achieved in mice with oral dosing that was well tolerated.

TABLE 6
Lung exposure of Oxy232 in mice

| | Mean Oxy232 Levels | SD | Mean Oxy232 Levels | SD |
|---|---|---|---|---|
| Time(h) | in Lung Tissue (ng/g) | (ng/mL) | in Lung Tissue (μM) | (μM) |
| 0 | 0 | 0 | 0 | 0 |
| 0.25 | 1500 | 96 | 3.41 | 0.22 |
| 0.5 | 8055 | 3226 | 18.32 | 7.34 |
| 1 | 9427 | 2826 | 21.44 | 6.43 |
| 2 | 7764 | 3141 | 17.66 | 7.14 |
| 4 | 2916 | 839 | 6.63 | 1.91 |
| 8 | 673 | 562 | 1.53 | 1.28 |
| 24 | 122 | 75 | 0.28 | 0.17 |

| | VeroE6 cells | VeroE6 cells | Calu-3 cells | Calu-3 cells |
|---|---|---|---|---|
| Oxy232 | (μM) | (ng/mL) | (μM) | (ng/ml) |
| IC₅₀ | 5.4 | 2374 | 6.9 | 3051 |
| IC₉₀ | 7.8 | 3429 | 13.5 | 5918 |

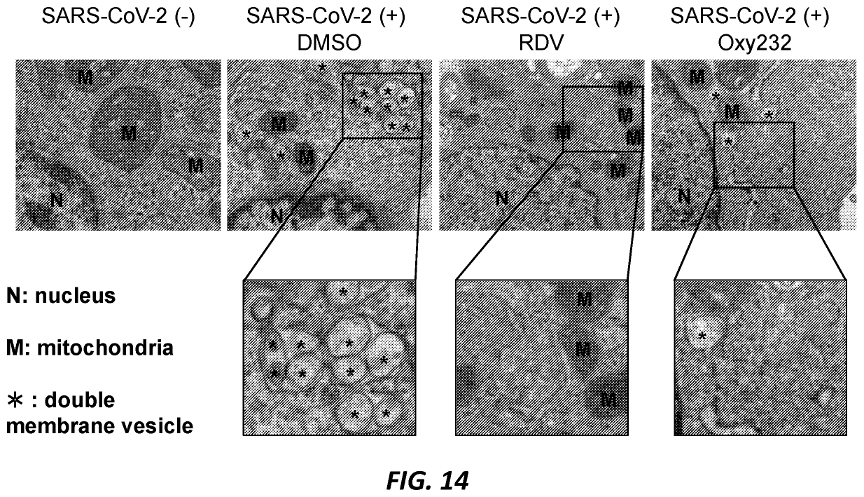

As noted earlier, members of several virus families, such as Arterviridae, Calicivridae, Flaviviridae, Picoronaviridae and Coronaviridae, which include all pathogenic coronaviruses and SARS-CoV-2, rely on DMVs for viral replication (Wolff G, Melia C E, Snijder E J, and Bárcena M. Double-Membrane Vesicles as Platforms for Viral Replication. Trends Microbiol. 2020; 28(12):1022-1033. PMCID: PMC7289118) After viral entry, coronaviruses induce the formation of perinuclear DMV clusters during the early stages of viral replication, mediated by membrane bound viral RNA synthesis. Inhibition of these processes with small molecules offers prospects for not only anti-SARS-CoV-2 activity but potentially broad antiviral activity against other pathogenic viruses (Wolff G, Melia C E, Snijder E J, and Bárcena M. Trends Microbiol. 2020; 28(12):1022-1033 and Garcia-Nicolas et al., The Small-Compound Inhibitor K22 Displays Broad Antiviral Activity against Different Members of the Family Flaviviridae and Offers Potential as a Panviral Inhibitor. Antimicrob Agents Chemother. 2018; 62(11): e01206-18. PMCID: PMC6201103) can inhibit the replication of SARS-CoV-2 and hepatitis C virus (HCV) which are DMV dependent, but not the replication of hepatitis D virus (HDV) whose replication is not DMV dependent (Ohashi et al., Int J Mol Sci. 2021; 22(6):3163 and Kong et al., Surfeit 4 Contributes to the Replication of Hepatitis C Virus Using Double-Membrane Vesicles. J Virol. 2020; 94(2): e00858-19. PMCID:). As shown in FIG. 14, these experiments were repeated with Oxy232. The data show that Oxy210 and Oxy232 inhibit DMV formation in virally infected host cells.

### Example 5: Anti-SARS-CoV-2 Activity of Exemplary Compounds of Formula (I)

### Materials and Methods

Cell culture: VeroE6/TMPRSS2 cells, VeroE6 cells overexpressing transmembrane protease, serine 2 (TMPRSS2), were cultured in DMEM supplemented with 10% fetal bovine serum, 100 units/mL penicillin, 100 μg/mL streptomycin, 10 mM HEPES (pH 7.4), and 1 mg/mL G418 at 37° C. in 5% CO₂. During the infection assay, 10% FBS was replaced with 2% FBS and G418 removed.

SARS-CoV-2 infection assay: SARS-CoV-2 was handled in a biosafety level 3 (BSL3). We used the SARS-CoV-2 Wk-521 strain, a clinical isolate from a COVID-19 patient, and obtained viral stocks by infecting VeroE6/TMPRSS2 cells (Matsuyama et al. Proc Natl Acad Sci USA. 2020). For the infection assay, VeroE6/TMPRSS2 cells were inoculated with virus at an MOI of 0.001 or 0.003 for 1 h and free virus removed by washing. Cells were cultured for additional 24 h to measure extracellular viral RNA or 48 h to observe cytopathic effects (CPE).

Quantification of viral RNA: Viral RNA was extracted with a QIAamp Viral RNA mini kit (QIAGEN) or MagMaX™ Viral/Pathogen II Nucleic Acid Isolation Kit (Thermo Fisher Scientific) and quantified by real time RT-PCR analysis with a one-step qRT-PCR kit (THUNDERBIRD Probe One-step qRT-PCR kit, TOYOBO) using 5′-ACAGGTACGTTAATAGTTAATAGCGT-3′ (SEQ ID NO: 7), 5′-ATATTGCAGCAGTACGCACACA-3′ (SEQ ID NO: 8), and 5′-FAM-ACACTAGCCATCCTTACTGCGCTTCG-TAMRA-3′ (E-set) (SEQ ID NO: 9) (Corman et al. Euro Surveill. 2020).

### Results and Discussion

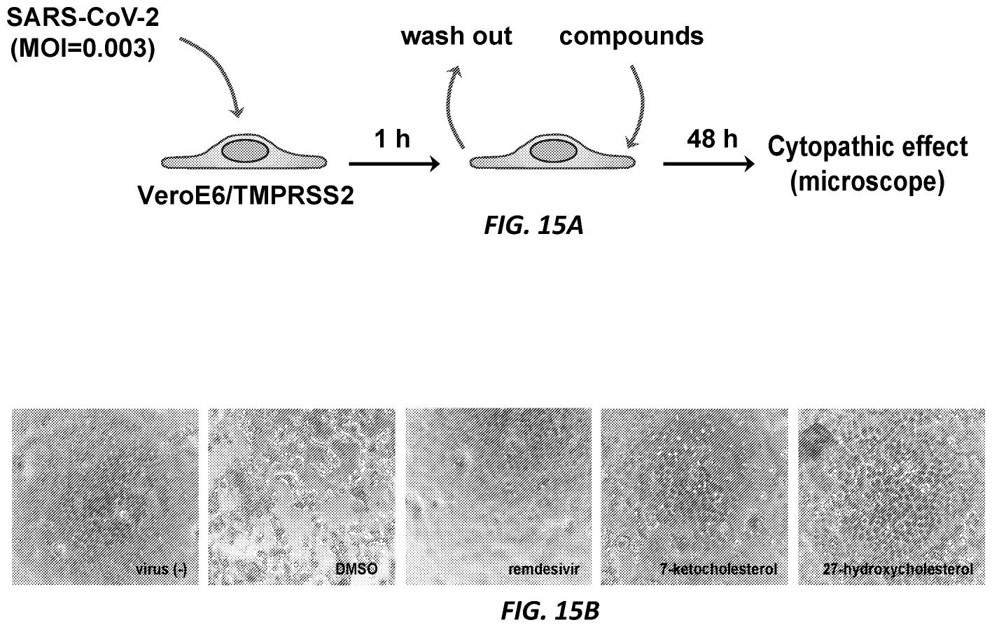

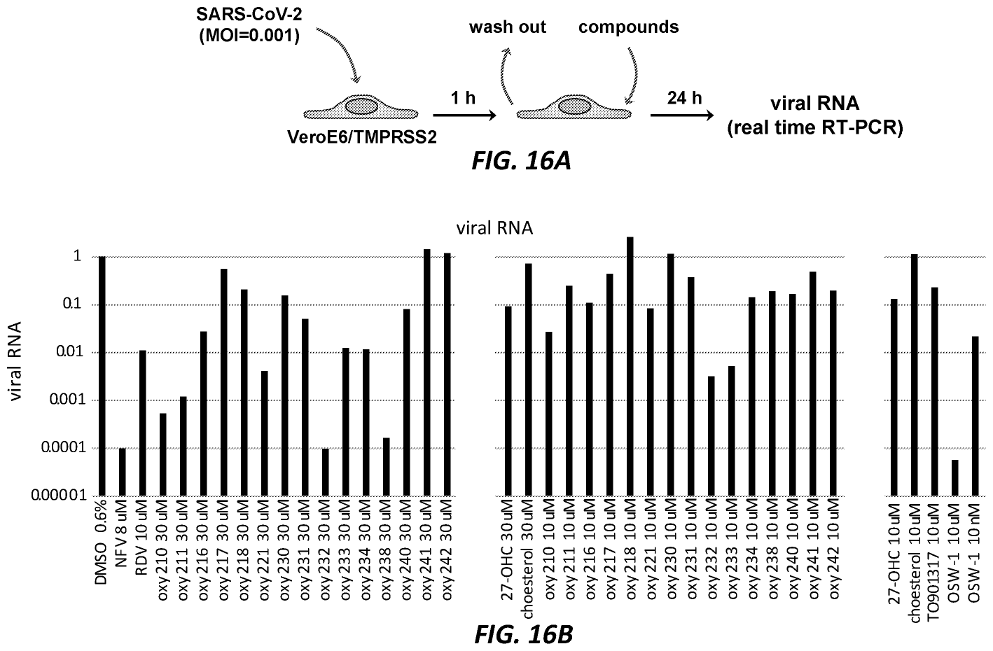

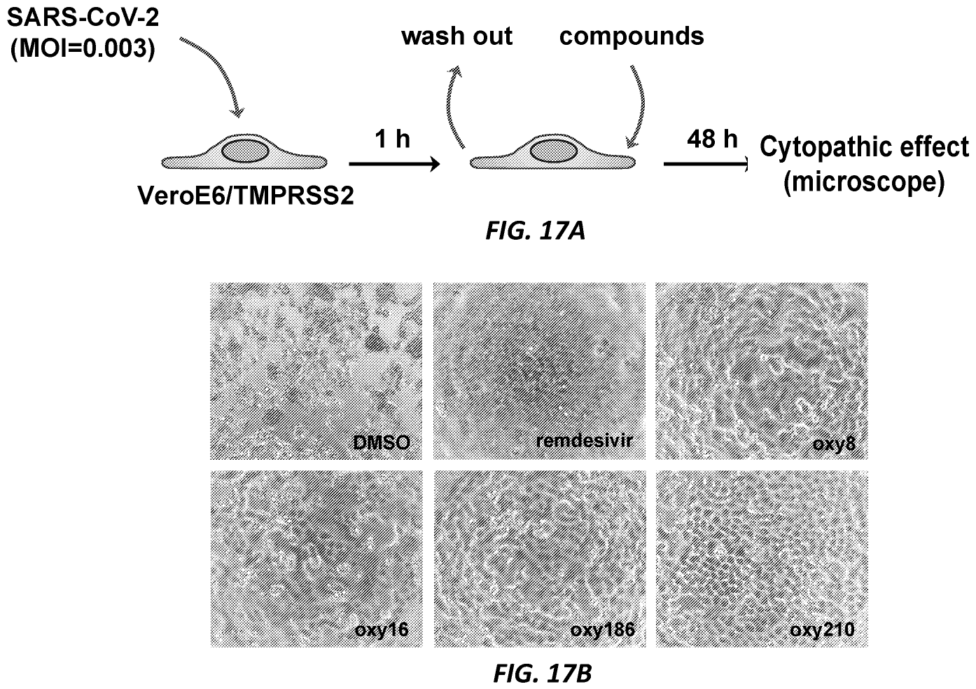

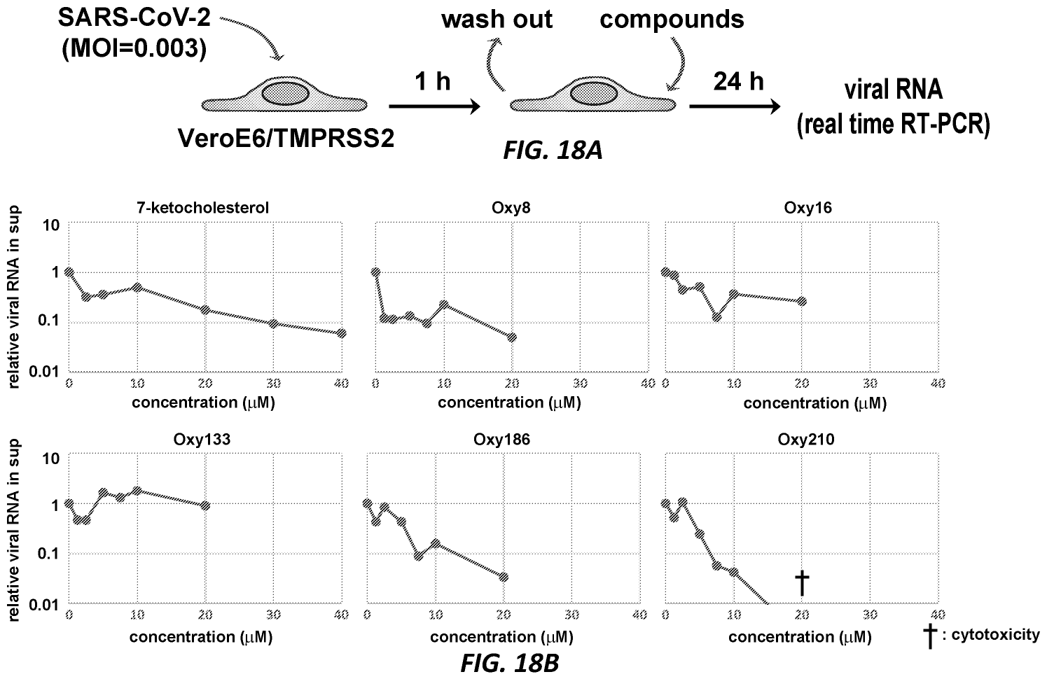

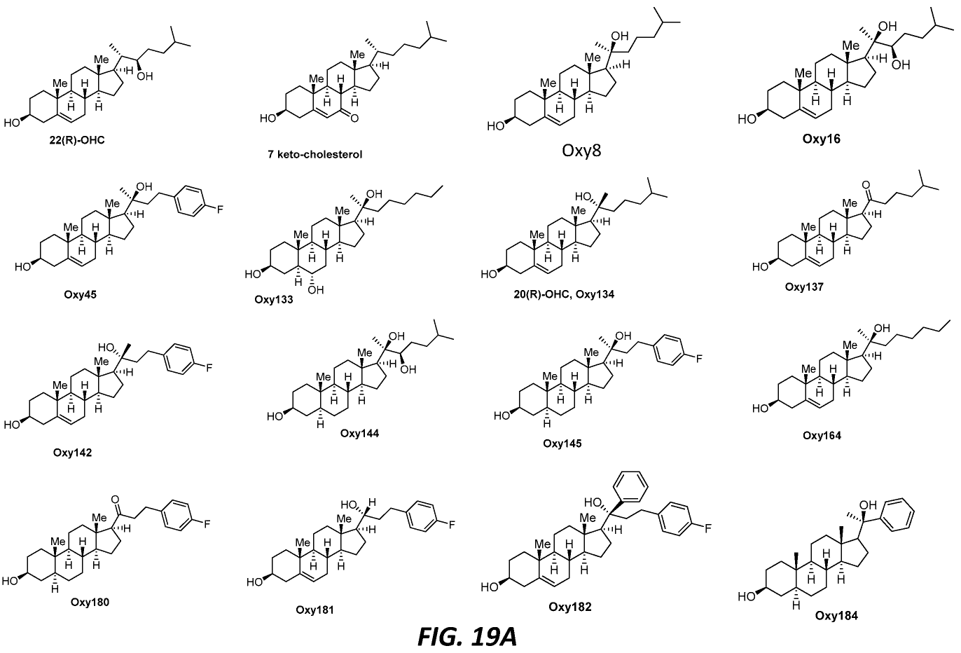

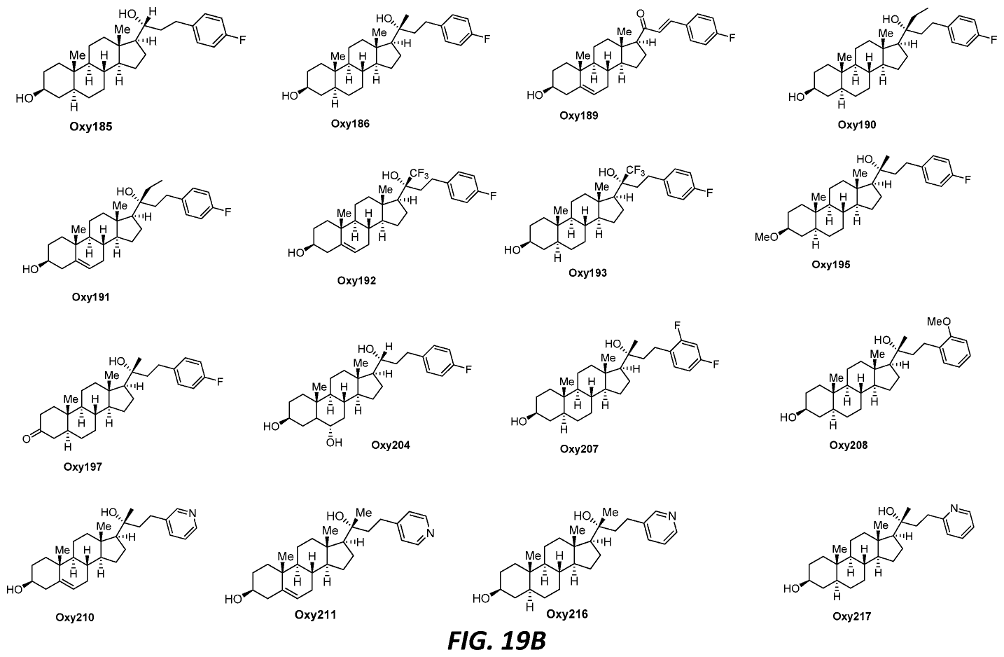

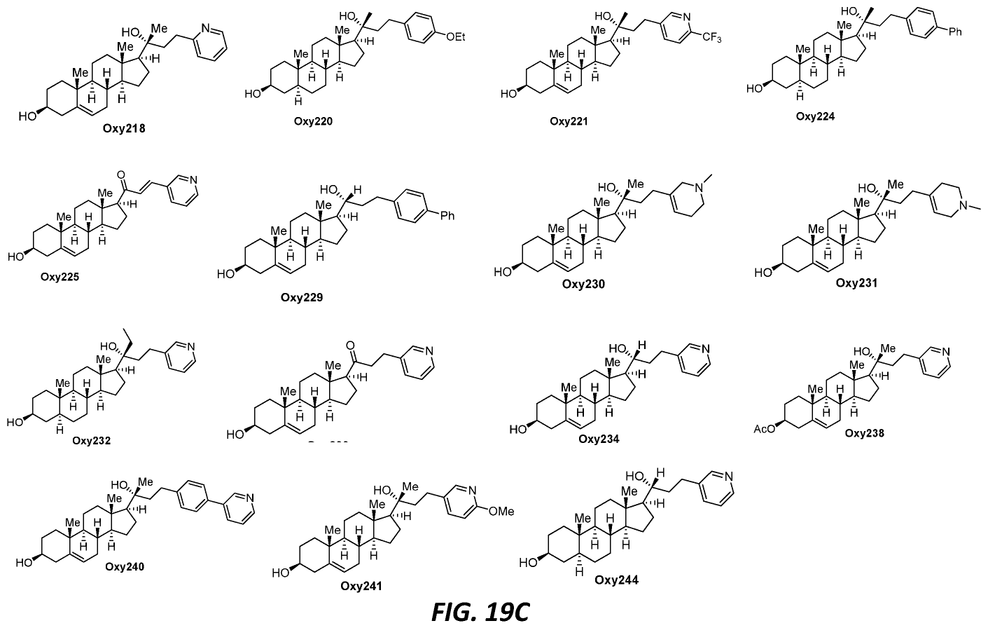

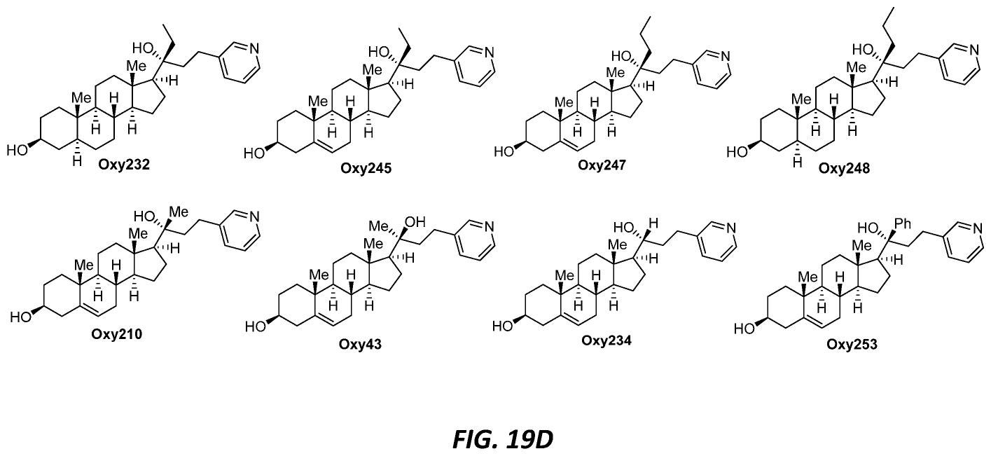

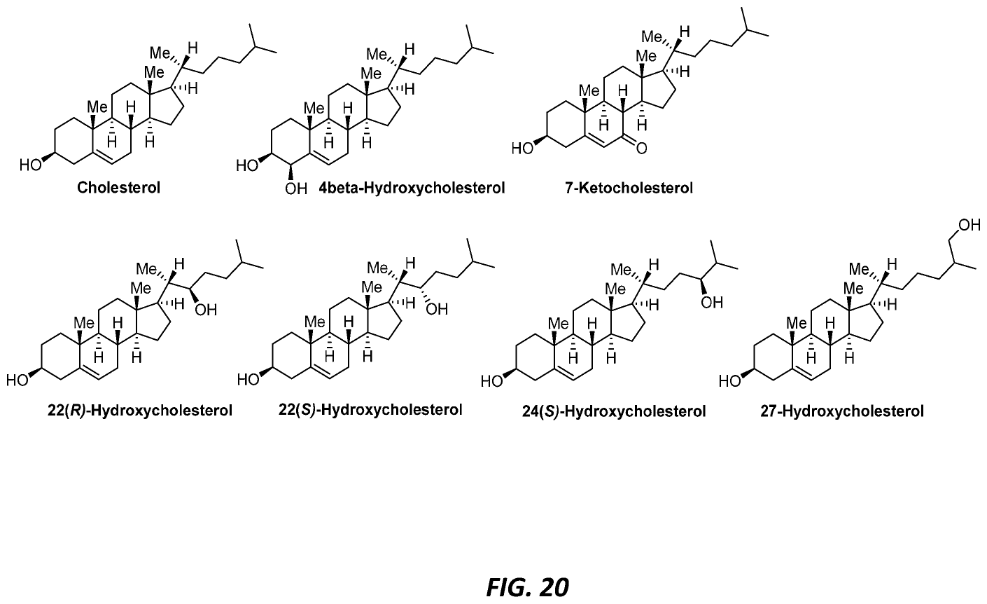

Results are shown in FIGS. 15B, 16B, 17B, and 18B.

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

1. A method for treating a viral infection, comprising administering a therapeutically effective amount of a compound of Formula (I), or a pharmaceutically acceptable salt or solvate thereof, to a subject in need thereof, wherein the compound of Formula (I) has the structure: 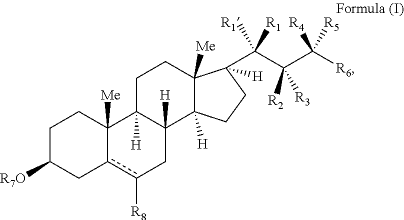
wherein: [symbol]
is a single or double bond;
R₁ and R₁′ are independently hydrogen, substituted or unsubstituted C₁-C₈ alkyl, substituted or unsubstituted C₁-C₈alkenyl, substituted or unsubstituted C₁-C₈alkynyl, substituted or unsubstituted aryl, substituted or unsubstituted heteroaryl, or substituted or unsubstituted —C₁-C₄alkylaryl, provided that one of R₁ and R₁′ is OH or R₁ and R₁′ together are ═O;
R₂, R₃, R₄, and R₅ are independently hydrogen, deuterium, C₁-C₈alkyl, or —OH, or one of R₂ or R₃ together with one of R₄ or R₅ forms a double bond;
R₆ is alkyl, aryl or heteroaryl, wherein the alkyl, aryl or the heteroaryl are optionally substituted with 1, 2, 3, or 4 R₉ groups;
R₇ is hydrogen, substituted or unsubstituted C₁-C₈alkyl, or —C(O)NR₁₀R₁₁;
R₈ is hydrogen or —OH;
each R₉ is independently selected from deuterium, halogen, —CN, C₁₋₆alkyl, C₂₋₆alkenyl, C₂₋₆alkynyl, C₃₋₄cycloalkyl, C₂₋₉heterocycloalkyl, C₆₋₁₀aryl, C₂₋₉heteroaryl, —OR₁₂, —SR₁₂, —N(R₁₃)(R₁₄), —C(O)OR₁₃, —C(O)N(R₁₃)(R₁₄), —C(O)R₁₅, —S(O)₂R₁₅, and —S(O)₂N(R₁₃)(R₁₄), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₂₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR₁₂, —SR₁₂, —N(R₁₃)(R₁₃), —C(O)OR₁₃, —C(O)N(R₁₃)(R₁₄), —C(O)R₁₅, —S(O)₂R₁₅, and —S(O)₂N(R₁₃)(R₁₄);
R₁₀ and R₁₁ are independently hydrogen, substituted or unsubstituted C₁-C₈alkyl, or substituted or unsubstituted aryl;
each R₁₂ is independently selected from H, C₁₋₆alkyl, C₁₋₆haloalkyl, C₃₋₆cycloalkyl, C₂₋₉heterocycloalkyl, C₆₋₁₀aryl, and C₁₋₉heteroaryl;
each R₁₃ and each R₁₄ are each independently selected from H, C₁₋₆alkyl, C₁₋₆haloalkyl, C₃₋₆cycloalkyl, C₂₋₉heterocycloalkyl, C₆₋₁₀aryl, and C₁₋₆heteroaryl; and
each R₁₅ is independently selected from C₁₋₆alkyl, C₁₋₆haloalkyl, C₃₋₆cycloalkyl, C₂₋₉heterocycloalkyl, C₆₋₁₀aryl, and C₁₋₉heteroaryl.

2. The method of claim 1, wherein the compound is an inhibitor of viral entry.

3. The method of claim 1, wherein the compound inhibits sodium taurocholate cotransport polypeptide (NTCP) oligomerization.

4. The method of claim 1, wherein the compound inhibits binding or interaction of NTCP with epidermal growth factor receptor (EGFR).

5. The method of claim 1, wherein the viral infection is selected from the group consisting respiratory infection, gastrointestinal infection, liver infection, nervous system infection, skin infection, placental infection and fetal viral infection.

6. The method of claim 1, wherein the viral infection is an infection of a tissue selected from the group consisting of lung tissue, upper respiratory system tissue, lower respiratory system tissue, central nervous system tissue, eye tissue, kidney tissue, bladder tissue, spleen tissue, cardiac tissue, gastrointestinal tissue, epidermal tissue, reproductive tissue, nasal cavity tissue, larynx tissue, trachea tissue, bronchi tissue, oral cavity tissue, and muscle tissue.

7. The method of claim 1, wherein the viral infection is by a DNA virus.

8. The method of claim 1, wherein the viral infection is by an RNA virus.

9. The method of claim 8, wherein the RNA virus is a positive strand RNA virus.

10. The method of claim 8, wherein the RNA virus is a negative strand RNA virus.

11. The method of claim 1, wherein the viral infection is by a virus from a virus family selected from the group consisting of abyssoviridae, ackermannviridae, adenoviridae, alloherpesviridae, alphaflexiviridae, alphasatellitidae, alphatetraviridae, alvernaviridae, amalgaviridae, amnoonviridae, ampullaviridae, anelloviridae, arenaviridae, arteriviridae, artoviridae, ascoviridae, asfarviridae, aspiviridae, astrovridae, autographiviridae, avsunviroidae, bacilladnaviridae, baculoviridae, barnaviridae, belpaovwridae, benyviridae, betaflexiviridae, bicaudaviridae, bidnaviridae, birnaviridae, bornaviridae, botourmiaviridae, bromoviridae, caliciviridae, carmotetraviridae, caulimoviridae, chaseviridae, chrysoviridae, chuviridae, circoviridae, clavaviridae, clostemviridae, coronaviridae, corticoviridae, cremegaviridae, cruliviridae, cystoviridae, deltaflexiviridae, demerecviridae, dicistroviridae, drexlerviridae, endornaviridae, euroniviridae, filoviridae, fimoviridae, finnlakeviridae, flaviviridae, fuselloviridae, gammaflexiviridae, geminiviridae, genomoviridae, globuloviridae, gresnaviridae, guttaviridae, halspiviridae, hantaviridae, hepadnaviridae, hepeviridae, herelleviridae, herpesviridae, hypoviridae, hytrosaviridae, flaviridae, inoviridae, iridoviridae, kitaviridae, lavidaviridae, leishbuviridae, leviviridae, lipothrixviridae, lispiviridae, luteoviridae, malacoherpesviridae, marnaviridae, marseilleviridae, matonaviridae, mayoviridae, medioniviridae, megabirnaviridae, mesoniviridae, metaviridae, microviridae, mimiviridae, mitoviridae, mononiviridae, mymonaviridae, myoviridae, mypowndae, nairoviridae, nanghoshaviridae, nanhypowndae, nanoviridae, narnaviridae, nimaviridae, nodaviridae, nudiviridae, nyamiviridae, ohfoviridae, orthomyxoviridae, ovaliviridae, papillomaviridae, pammyxoviridae, partitiviridae, parvoviridae, peribunyaviridae, permutotetraviridae, phasmaviridae, phenuiviridae, phycodnaviridae, picobirnaviridae, picornaviridae, plasmaviridae, plectroviridae, pleolipoviridae, pneumoviridae, podoviridae, polycipiviridae, polydnaviridae, polymycoviridae, polyomaviridae, portoglobovridae, pospiviroidae, potyviridae, poxviridae, pseudoviridae, qinviridae, quadriviridae, redondoviridae, reoviridae, retroviridae, rhabdoviridae, roniviridae, rudiviridae, sarthroviridae, secoviridae, sinhaliviridae, siphoviridae, smacoviridae, solemoviridae, solinviviridae, sphaerolipoviridae, spiraviridae, sunviridae, tectiviridae, thaspiviridae, tobaniviridae, togaviridae, tolecusatellitidae, tombusviridae, tospoviridae, totiviridae, tristromaviridae, turriviridae, tymoviridae, virgaviridae, wupedeviridae, xinmoviridae, and yueviridae

12. The method of claim 1, wherein the viral infection is by a virus selected from the group consisting of hepadnaviruses, coronaviruses, avian influenza viruses, adenoviruses, herpesviruses, human papillomaviruses, parvoviruses, reoviruses, picornaviruses, flaviviruses, togaviruses, orthomyxovirus, bunyaviruses, rhabdoviruses, and paramyxoviruses.

13. The method of claim 1, wherein the viral infection is a hepatitis B virus (HBV) infection.

14. The method of claim 1, wherein the viral infection is a coronavirus infection.

15. The method of claim 14, wherein the coronavirus is selected from the group consisting of: severe acute respiratory syndrome-associated coronavirus (SARS-CoV); severe acute respiratory syndrome-associated coronavirus 2 (SARS-CoV-2); Middle East respiratory syndrome-related coronavirus (MERS-CoV); HCoV-NL63; and HCoV-HKu1.

16. The method of claim 15, wherein the coronavirus is SARS-CoV-2.

17. The method of claim 1, wherein the infection is a human immunodeficiency virus (HIV) infection.

18. The method of claim 1, wherein the viral infection is a latent viral infection.

19. The method of claim 1, wherein the administration is systemic.

20. The method of claim 1, wherein the administration is local at a site of viral infection.

21. The method of claim 1, further comprising administering at least one additional therapeutic to the subject.

22. The method of claim 21, wherein the at least one additional therapeutic is an anti-viral therapeutic.

23. The method of claim 22, wherein the anti-viral therapeutic is selected from the group consisting of Abacavir, Acyclovir (Aciclovir), Adefovir, Amantadine, Ampligen, Amprenavir (Agenerase), Arbidol, Atazanavir, Atripla, Balavir, Baloxavir marboxil (Xofluza®), Biktarvy Boceprevir (Victrelis®), Cidofovir, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Fomivirsen, Fosamprenavir, Foscamet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Lamivudine, Letermovir (Prevymis®), Lopinavir, Loviride, Maraviroc, Methisazone, Moroxydine, Nelfinavir, Nevirapine, Nexavir®, Nitazoxanide, Norvir, Nucleoside analogues, Oseltamivir (Tamiflu®), Peginterferon alfa-2a, Peginterferon alfa-2b, Penciclovir, Peramivir (Rapivab®), Pleconaril, Podophyllotoxin, Protease inhibitor (pharmacology), Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Telaprevir, Telbivudine (Tyzeka®), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza®), and Zidovudine.

24-25. (canceled)

26. A method for inhibiting viral entry into a cell, comprising administering to the cell a compound of Formula (I) or a pharmaceutically acceptable salt or solvate thereof.

27. The method of claim 26, wherein said administering to the cell is in vivo.

28. The method of claim 26, wherein said administering to the cell is in a subject having a viral infection.

29. The method of claim 1, wherein the compound is of Formula (Ia):

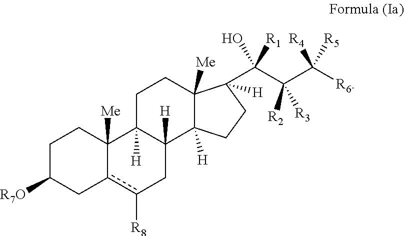

30. The method of claim 1, wherein the compound is of Formula (II):

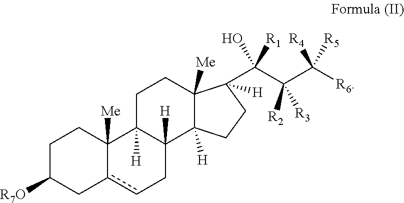

31. The method of claim 1, wherein the compound is of Formula (IIa):

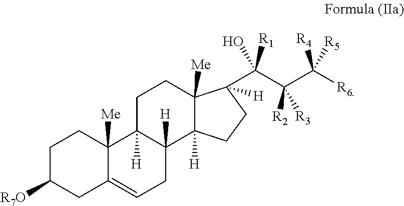

32. The method of claim 1, wherein the compound is of Formula (IIb):

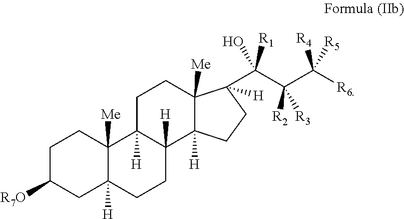

33. The method of claim 1, wherein the compound is of Formula (I): 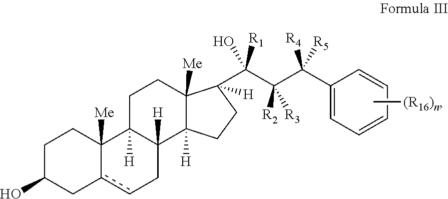
wherein:
each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl; and
n is 0, 1, 2, or 3.

34. The method of claim 33, wherein the compound is of Formula (IIIa): 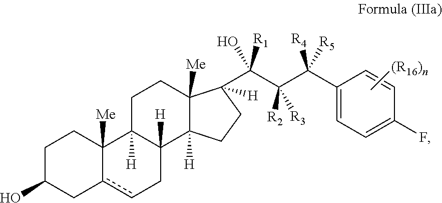
wherein:
n is 0, 1 or 2.

35. The method of claim 1, wherein the compound is of Formula (IV): 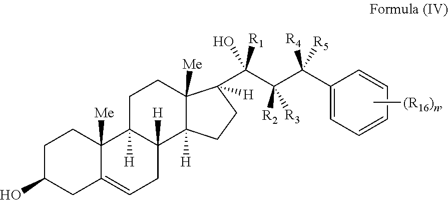
wherein:
each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl; and
n is 0, 1, 2, or 3.

36. The method of claim 35, wherein the compound is of Formula (IVa): 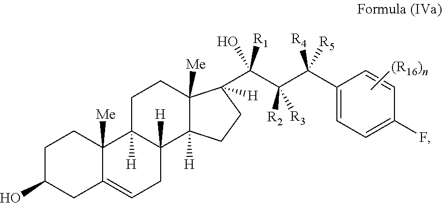
wherein:
n is 0, 1 or 2.

37. The method of claim 1, wherein the compound is of Formula (V): 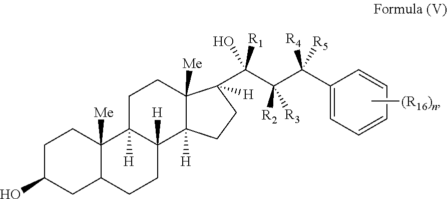
wherein:
each R₁₆ is independently halogen, hydroxy, substituted or unsubstituted C₁-C₈alkyl, substituted or unsubstituted C₁-C₈alkoxy, substituted or unsubstituted C₁-C₈heteroalkyl, substituted or unsubstituted C₃-C₈cycloalkyl, substituted or unsubstituted aryl, or substituted or unsubstituted heteroaryl; and
n is 0, 1, 2, or 3.

38. The method of claim 37, wherein the compound is of Formula (Va): 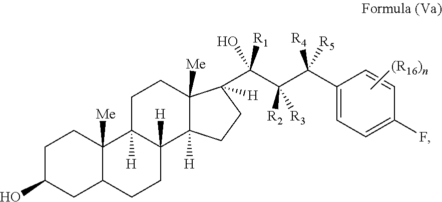
wherein:
n is 0, 1 or 2.

39. The method of claim 1, wherein the compound is of Formula (VI):

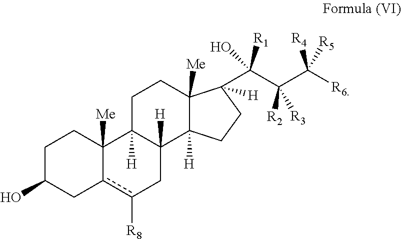

40. The method of claim 39, wherein the compound is of Formula (VIa):

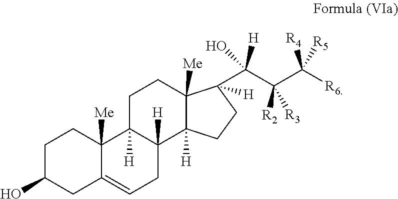

41. The method of claim 39, wherein the compound is of Formula (VIb):

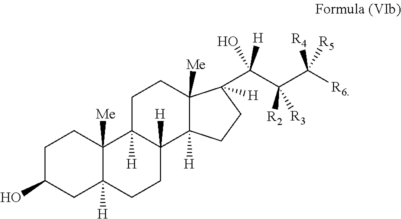

42. The method of claim 39, wherein the compound is of Formula (VIc):

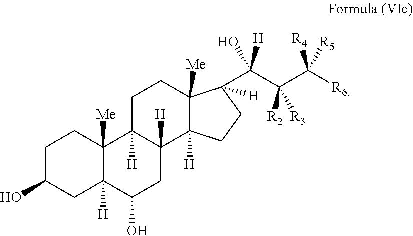

43. The method of claim 1, wherein the compound is of Formula (VII):

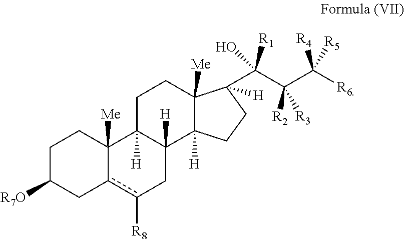

44. The method of claim 43, wherein the compound is of Formula (VIIa):

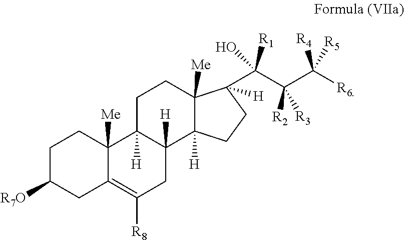

45. The method of claim 43, wherein the compound is of Formula (VIIb):

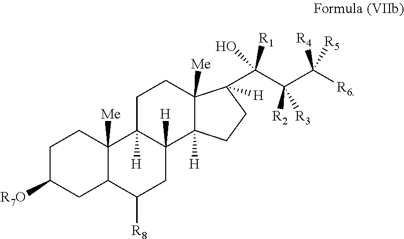

46. The method of claim 1, wherein the compound is selected from the group consisting of: 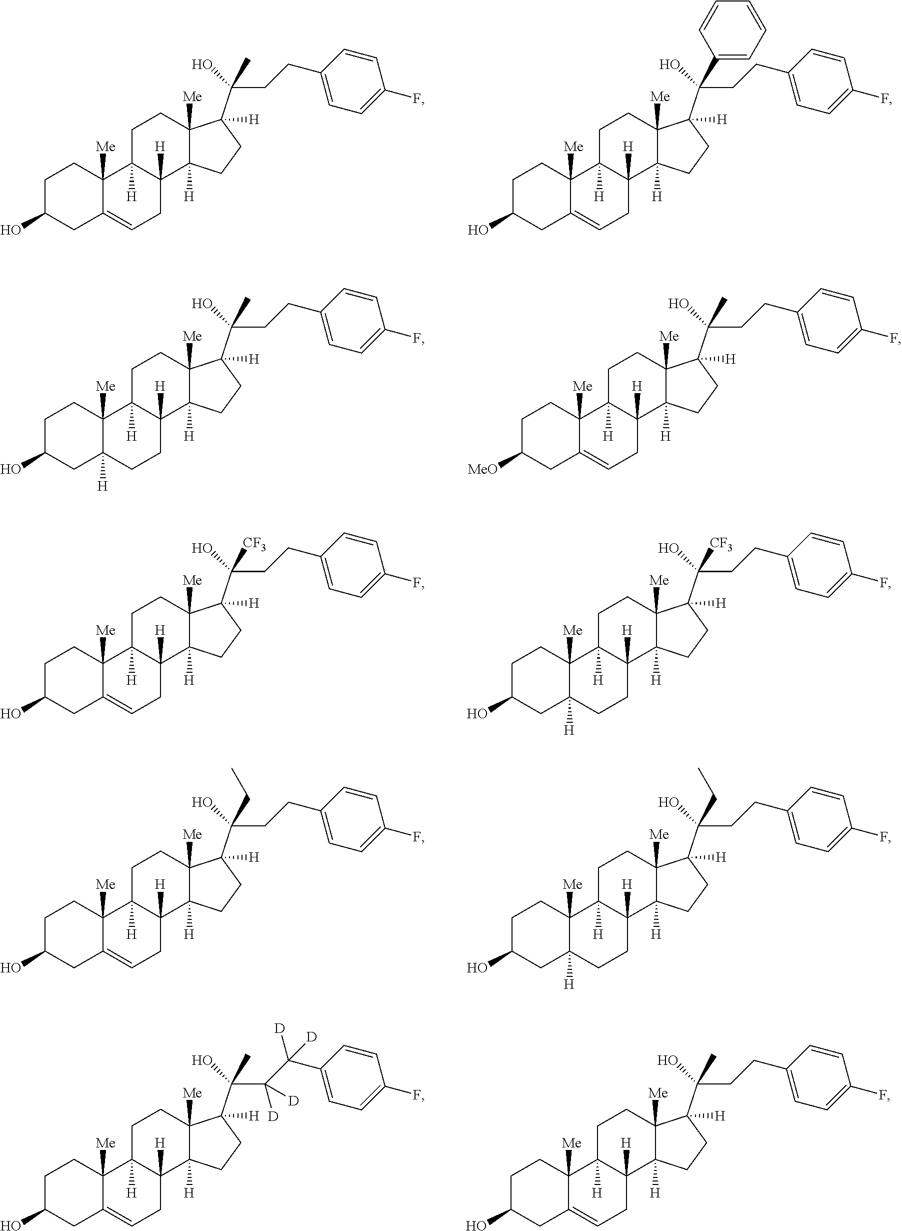 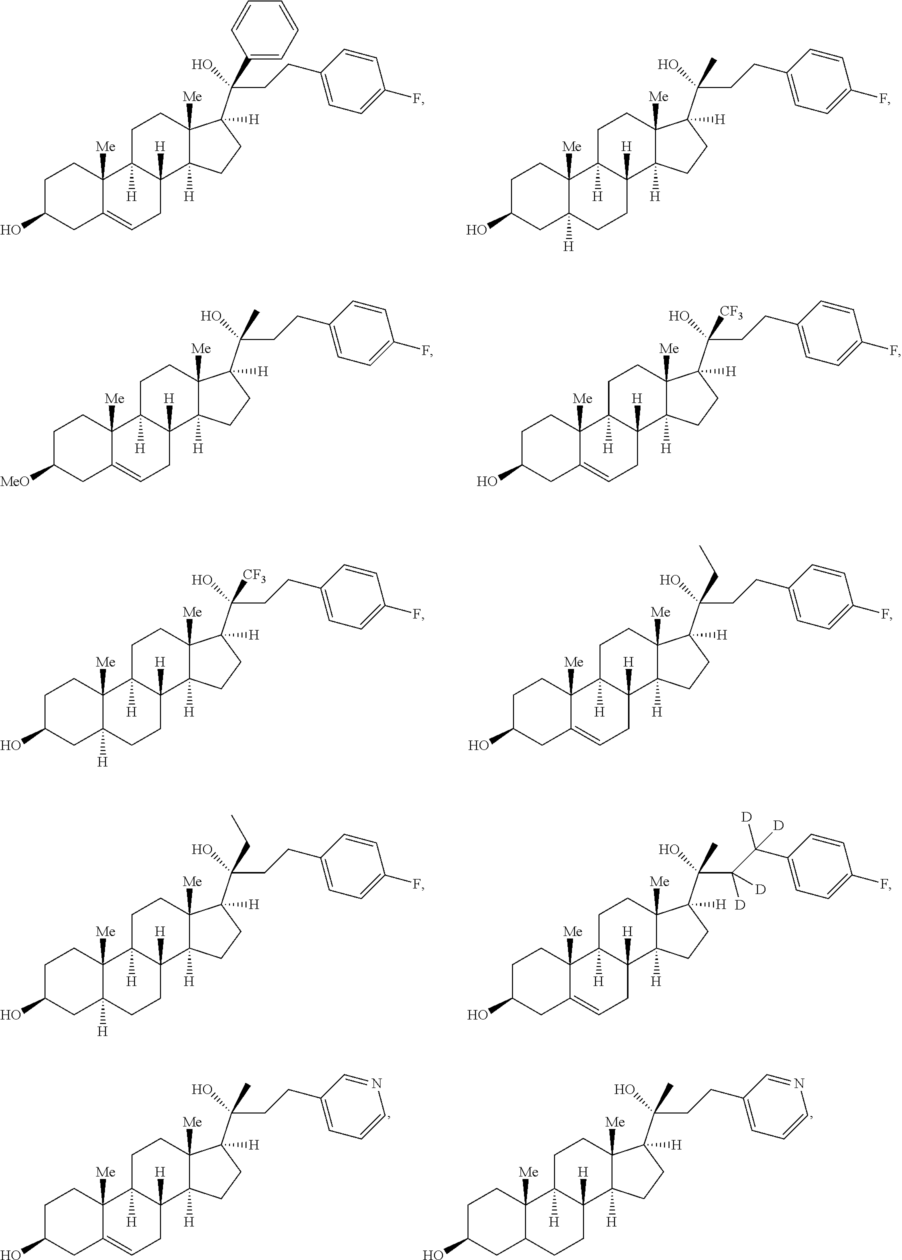 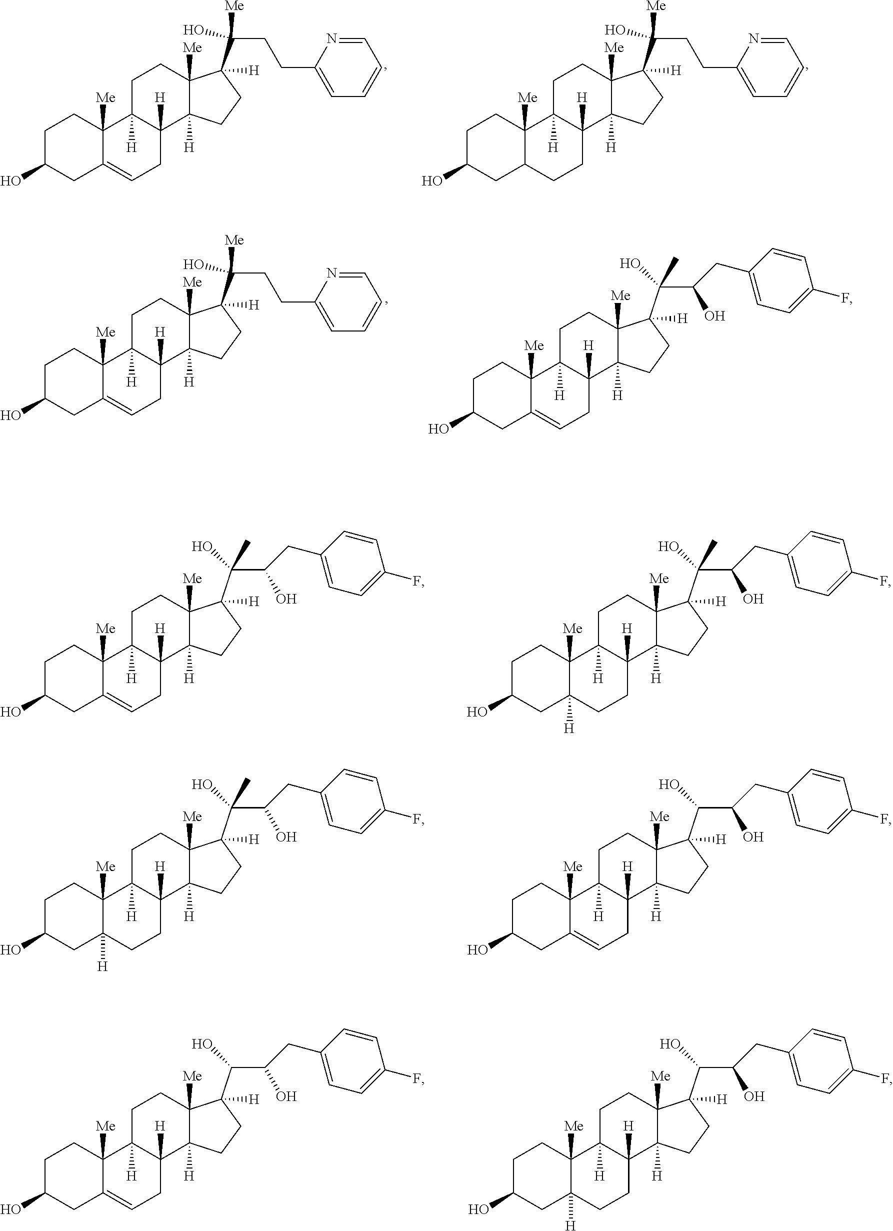 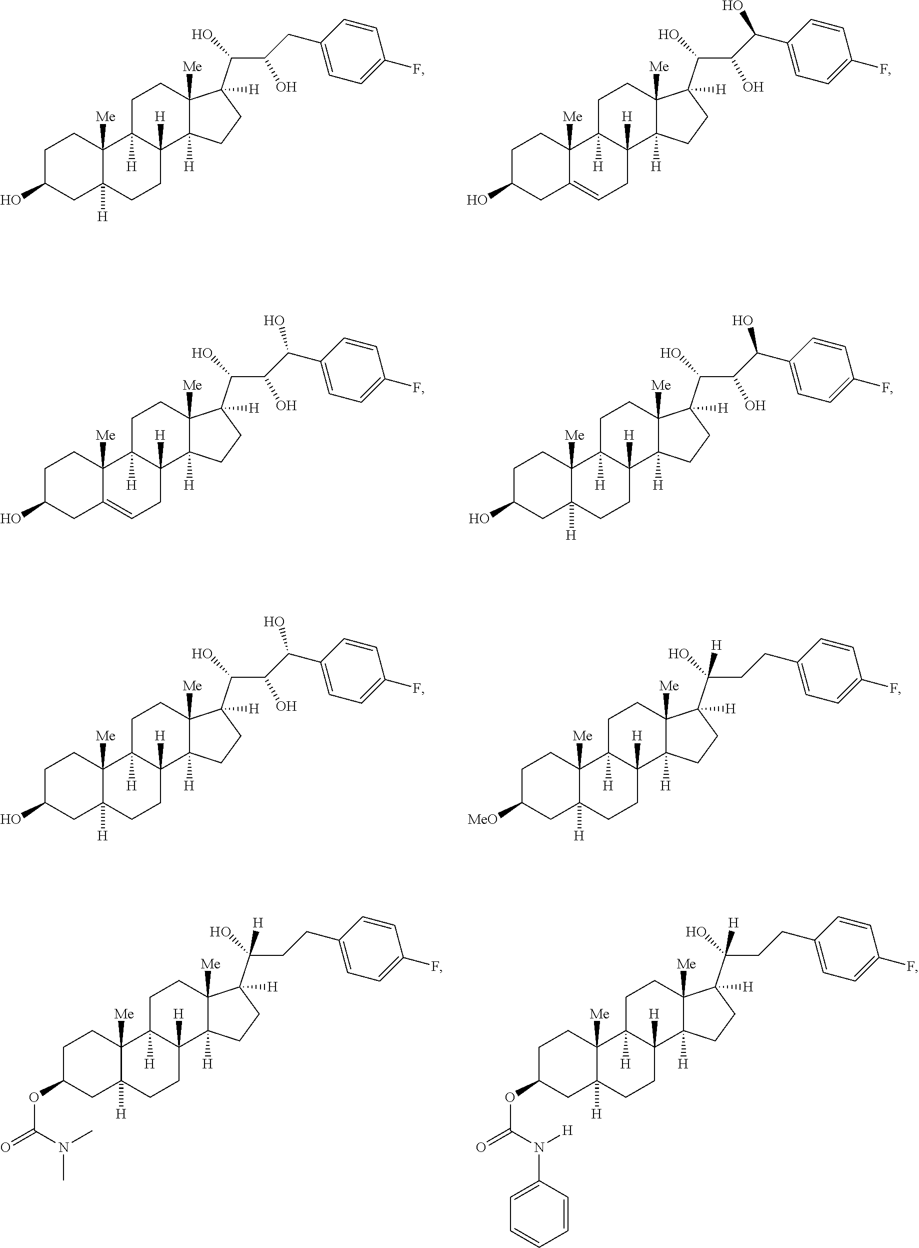 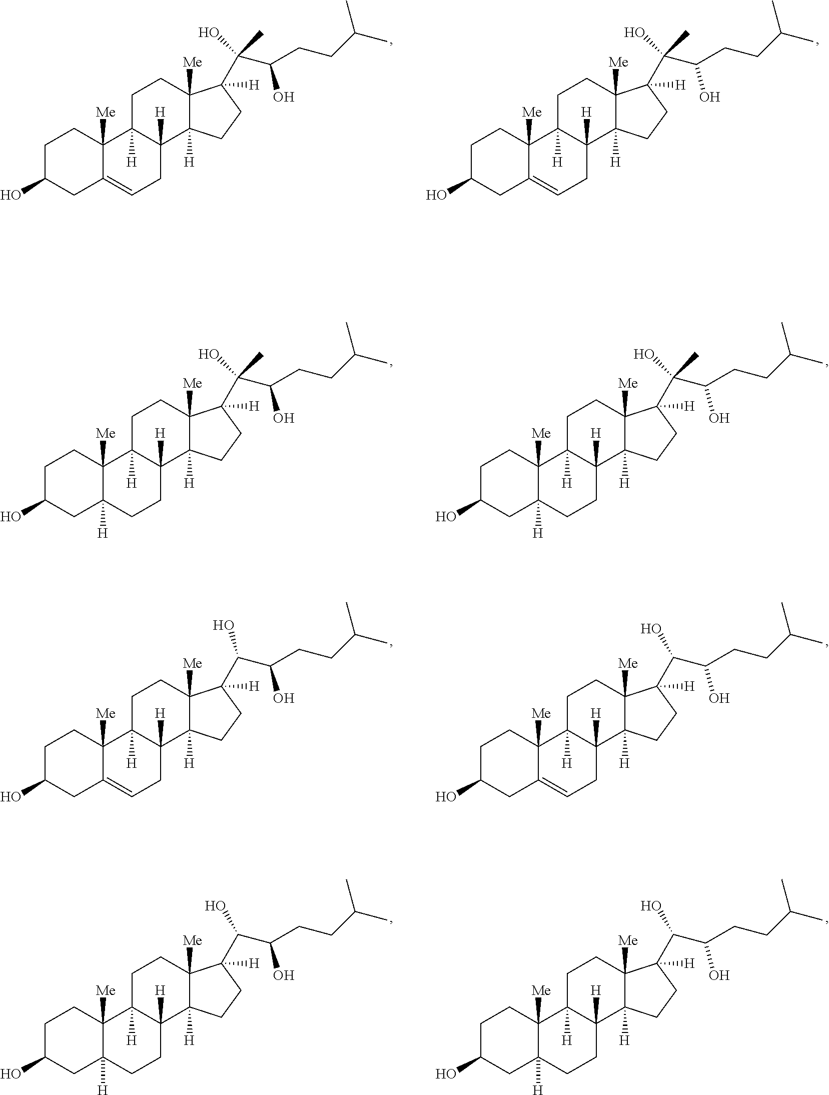 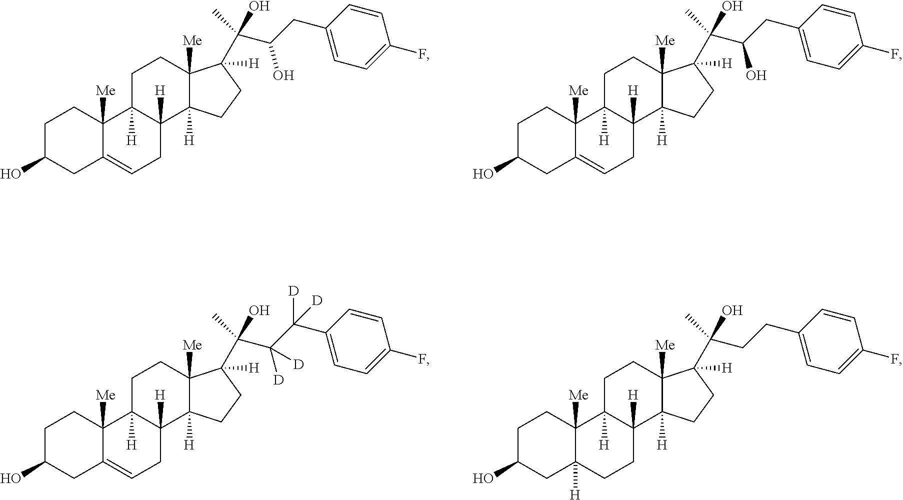 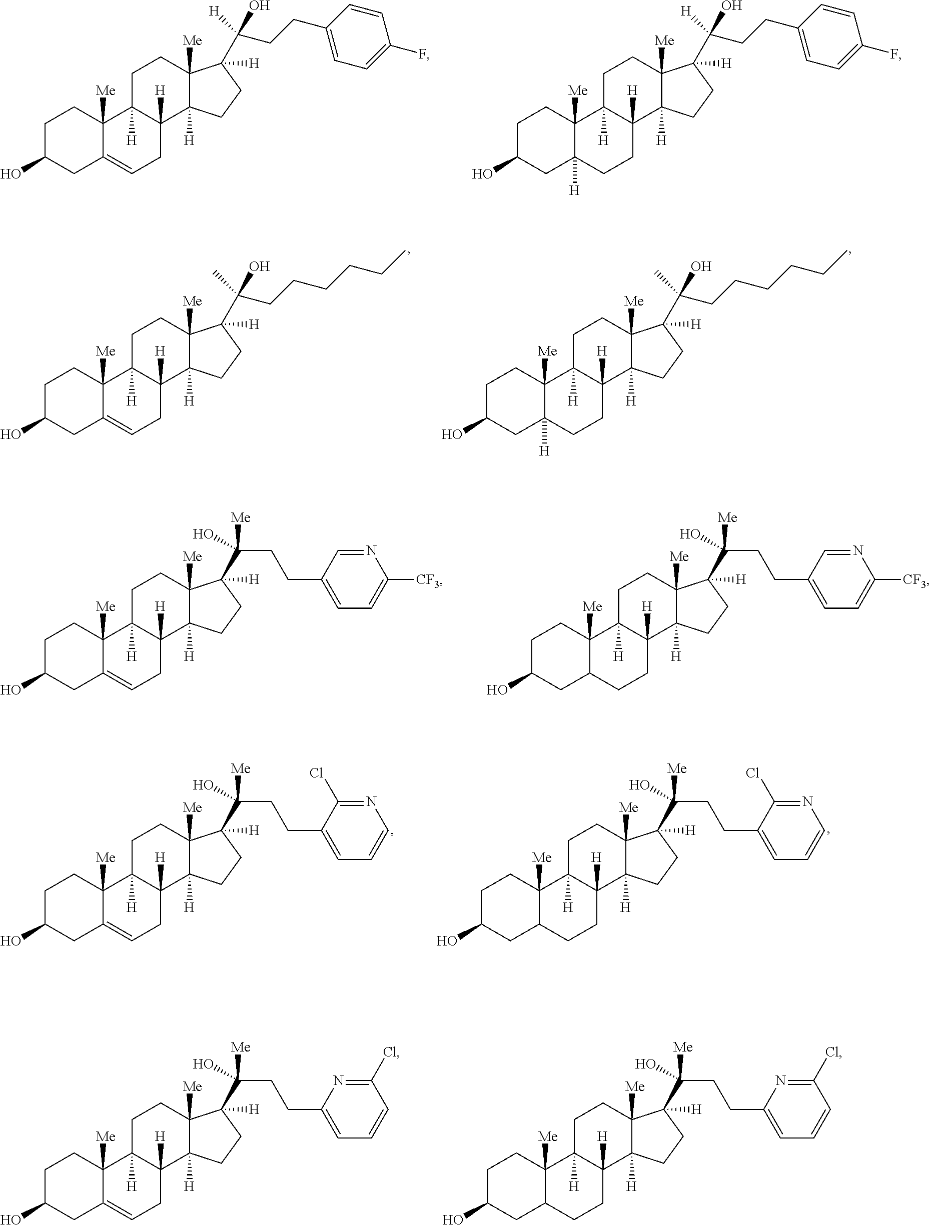 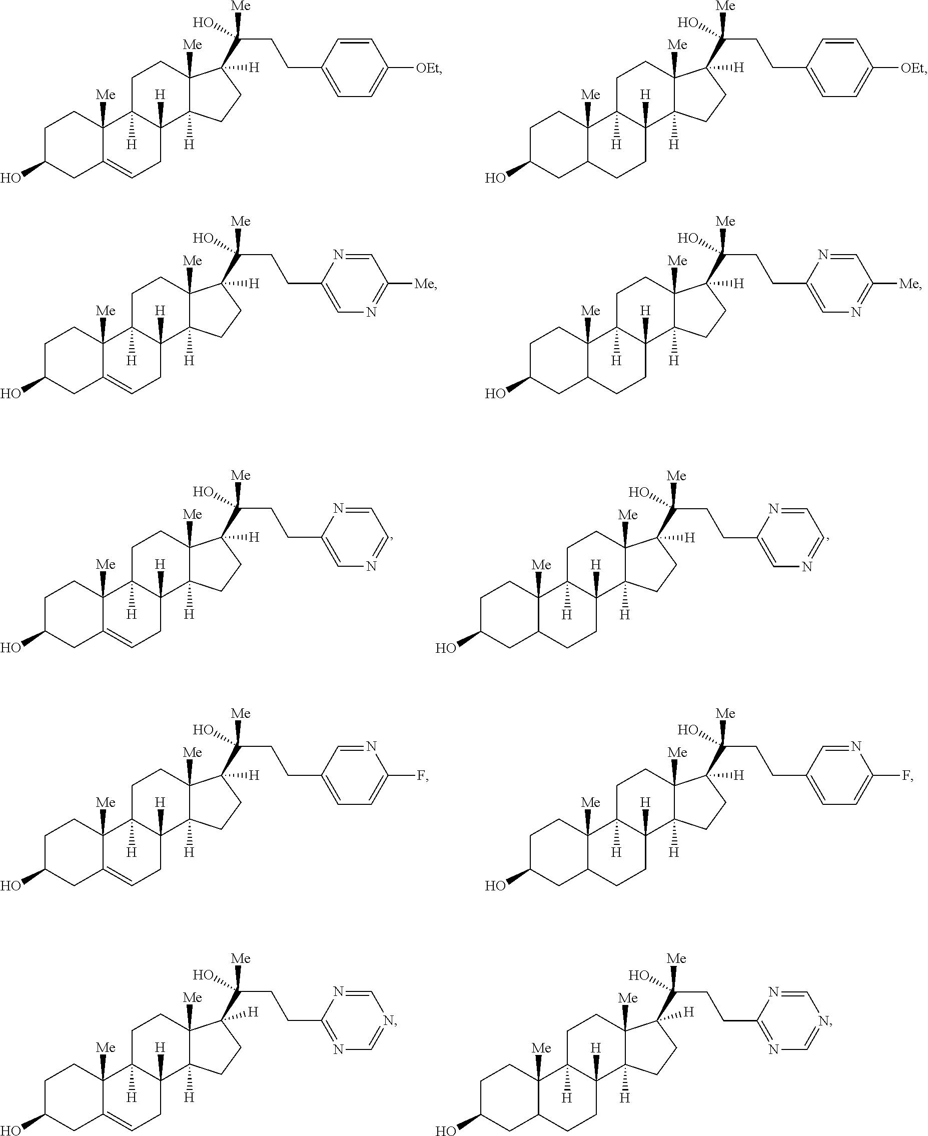 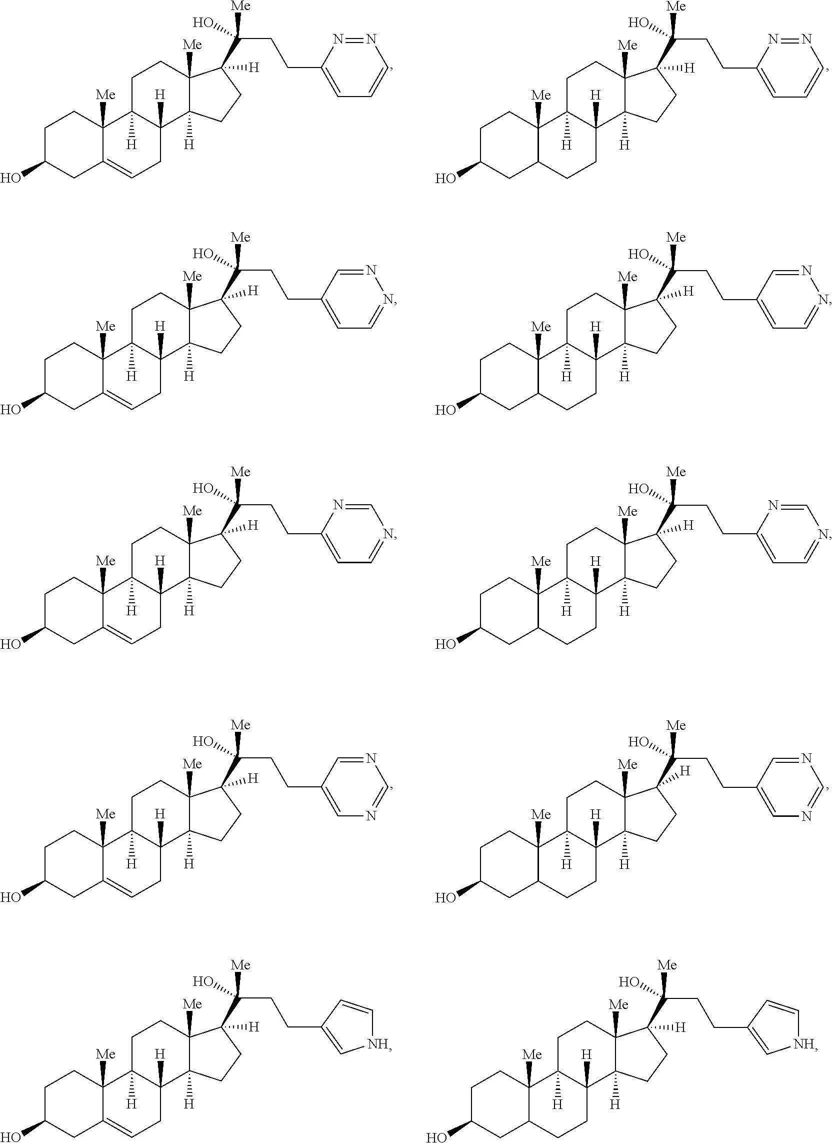 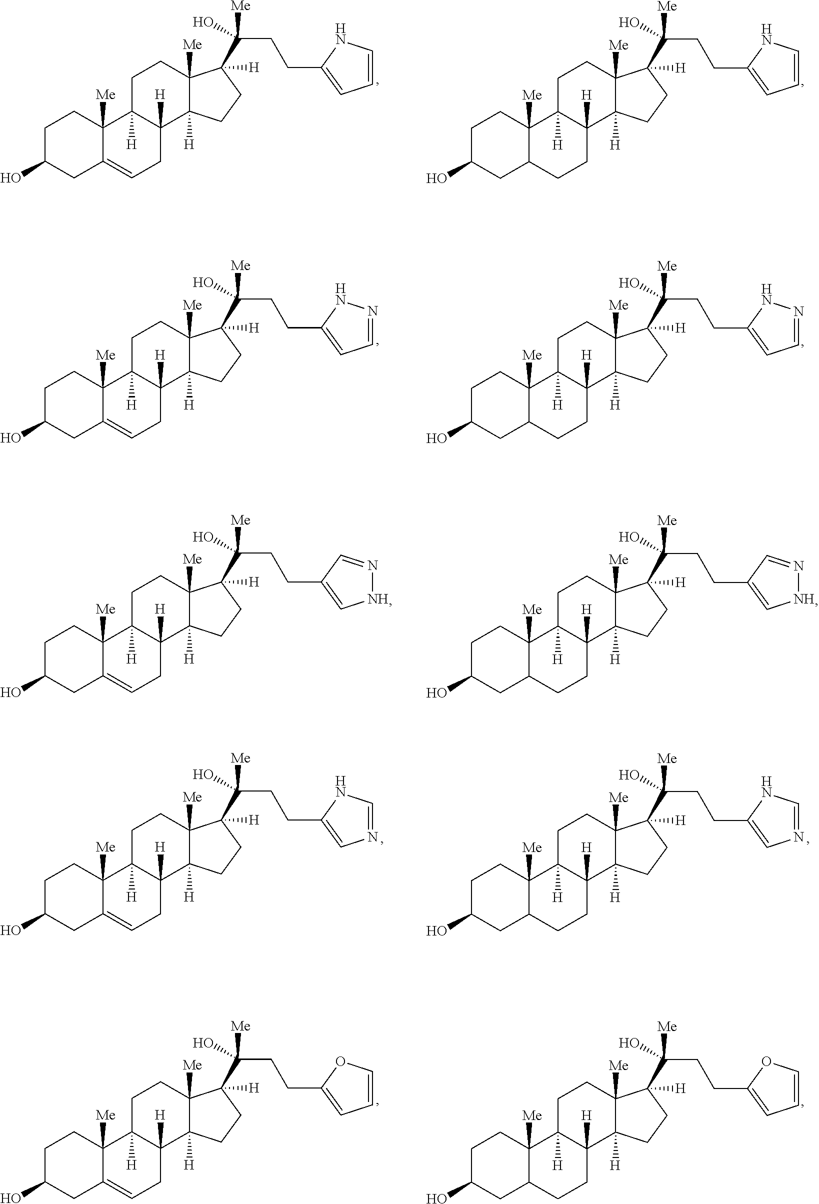 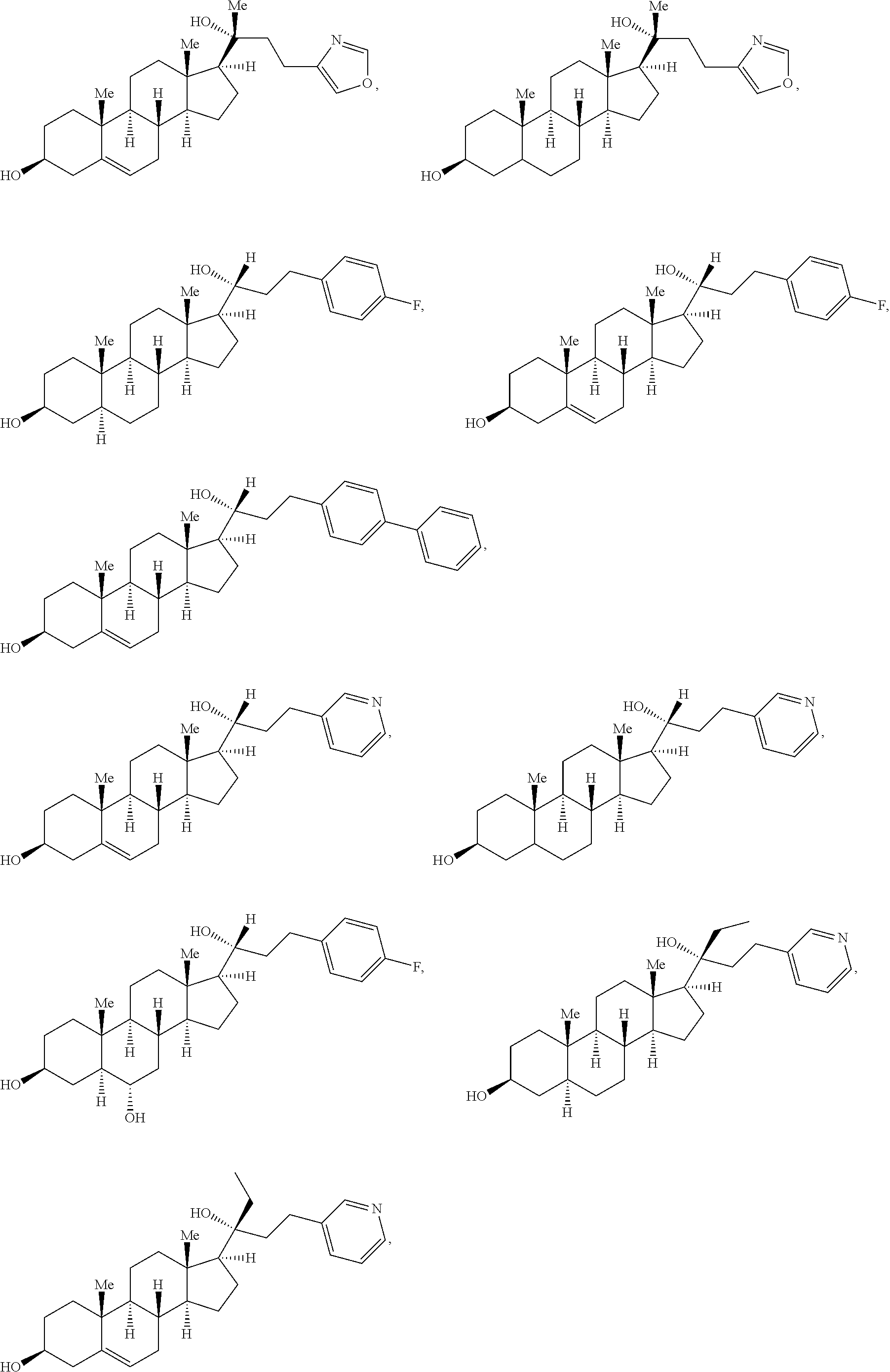 [chemical structure]


SEQUENCE LISTING (9 sequences)

SEQ ID NO: 1 - DNA, 22 bp, Artificial Sequence
  Description of Artificial Sequence Synthetic oligonucleotide
aaggtaggagctggagcattcg

SEQ ID NO: 2 - DNA, 20 bp, Artificial Sequence
  Description of Artificial Sequence Synthetic oligonucleotide
aggcggatttgctggcaaag

SEQ ID NO: 3 - DNA, 22 bp, Artificial Sequence
  Description of Artificial Sequence Synthetic oligonucleotide
agccctcaggctcagggcatac

SEQ ID NO: 4 - DNA, 21 bp, Artificial Sequence
  Description of Artificial Sequence Synthetic oligonucleotide
cgtctgtgccttctcatctgc

SEQ ID NO: 5 - DNA, 20 bp, Artificial Sequence
  Description of Artificial Sequence Synthetic oligonucleotide
gcacagcttggaggcttgaa

SEQ ID NO: 6 - DNA, 18 bp, Artificial Sequence
  Description of Artificial Sequence Synthetic oligonucleotide
ctgtaggcataaattggt

SEQ ID NO: 7 - DNA, 26 bp, Artificial Sequence
  Description of Artificial Sequence Synthetic oligonucleotide
acaggtacgttaatagttaatagcgt

SEQ ID NO: 8 - DNA, 22 bp, Artificial Sequence
  Description of Artificial Sequence Synthetic oligonucleotide
atattgcagcagtacgcacaca

SEQ ID NO: 9 - DNA, 26 bp, Artificial Sequence
  Description of Artificial Sequence Synthetic oligonucleotide
acactagccatccttactgcgcttcg